(19) United States
(12) Patent Application Publication
(10) Pub. No.: US 2024/0219375 A1
(43) Pub. Date: Jul. 4, 2024

(54) DOPAMINERGIC PRECURSOR CELLS AND METHODS OF USE

(71) Applicant: FUJIFILM Cellular Dynamics, Inc., Madison, WI (US)

(72) Inventors: Christopher McMAHON, Madison, WI (US); Randall LEARISH, Madison, WI (US); Carrie CHAVEZ, Madison, WI (US); Cayla THOMPSON, Madison, WI (US)

(73) Assignee: FUJIFILM Cellular Dynamics, Inc., Madison, WI (US)

(21) Appl. No.: 18/554,103

(22) PCT Filed: Apr. 7, 2022
(86) PCT No.: PCT/US2022/023797

§ 371 Date: Oct. 5, 2023

Related U.S. Application Data
(60) Provisional application No. 63/275,691, filed on Nov. 4, 2021.
(60) Provisional application No. 63/171,837, filed on Apr. 7, 2021.

(51) Int. Cl. G01N 33/50; C12N 5/0793

(52) U.S. Cl. CPC G01N 33/5058; C12N 5/0619; G01N 33/502; C12N 2501/119; C12N 2501/13; C12N 2501/15; C12N 2501/41; C12N 2501/415; C12N 2506/45; G01N 2500/10

(57) ABSTRACT

Midbrain dopaminergic neuronal precursor cells that can be used to treat a brain disorder are provided herein. Improved mono-SMAD methods are provided that can be used to differentiate pluripotent cells into midbrain dopaminergic (DA) neurons or midbrain neuronal precursors. In some aspects, methods are provided for mono-SMAD culture protocols and culture durations that can be used to generate dopaminergic neuronal precursor cells that have significantly improved properties for the treatment of a brain disorder such as, e.g., Parkinson's disease. Methods of treating Parkinson's disease and other brain diseases with the midbrain dopaminergic neuronal precursor cells are also provided.

### PRIORITY CLAIM

This application claims benefit of priority to U.S. Provisional Application Ser. No. 63/171,837, filed Apr. 7, 2021, and U.S. Provisional Application Ser. No. 63/275,691, filed Nov. 4, 2021, the entire contents of which are hereby incorporated by reference.

### BACKGROUND OF THE INVENTION

### 1. Field of the Invention

The present invention relates generally to the field of molecular biology and medicine. More particularly, it concerns methods of producing neuronal precursor cells from pluripotent stem cells and related methods of treatment.

### 2. Description of Related Art

Parkinson's Disease (PD) is a debilitating neurodegenerative disease presenting as a movement disorder due to the loss of A9 midbrain dopaminergic (mDA) neurons and subsequent loss of dopamine neuronal signaling. Current PD treatments including dopaminergic drug therapy and deep brain stimulation (DBS) address motor symptom improvement.

The clinical and social cost of PD is predicted to rise, and current treatment options exhibit significant limitations. As the population continues to age, PD are expected to dramatically rise, with conservative estimates of over 14 million victims globally by 2040 (Dorsey & Bloem, 2018). Although PD patients can display a range of non-motor features, the defining symptoms are progressive motor deficits due to striatal dopaminergic insufficiency secondary to loss of dopaminergic nigral neurons. Current therapies are symptomatic, mostly focused on ameliorating motor deficits.

Dopamine agonist delivery typically provides only mid-to-moderate relief. Treatment with L-Dopa requires careful dose management, is usually only effective for 4-6 years, and often leads to dyskinesias. For example, oral L-DOPA or dopaminergic agonists may initially provide relief from motor symptoms, but after 5-10 years most patients experience debilitating motor fluctuations and dyskinesias (Ahlskog & Muenter, 2001). DBS requires the use of invasive implants, has known neuropsychiatric side effects, and is typically effective for less than 10 years. DBS stimulation of the subthalamic nucleus (STN) or internal segment of the globus pallidus can compensate for DA loss in some patients, but this approach is primarily indicated for younger patients who do not display cognitive decline and periodic battery changes are required. None of these treatments address the underlying disease pathology, the progressive loss of mDA neurons.

Although cell transplantation therapies have been tested, significant limitations have been associated with these efforts. A cell-based therapy to replace lost cells and provide relief from PD motor symptoms for 10-15 years is a major goal for the treatment of PD. Studies using fetal tissue have been performed (Brundin et al., 1986; Doucet et al., 1989; Freeman, Sanberg, et al., 1995). Studies using fetal ventral mesencephalon (fVM) cells as the source of midbrain neural progenitors have been performed (Barker et al., 2013); however, efficacy results among several different studies involving transplantation of cells has been mixed or inconsistent (e.g., Barbuti et al., 2021; Barker, Drouin-Ouellet, & Parmar, 2015).

Replacement therapies involving administration to fVM cells exhibit both ethnical and technical limitations. Dopamine neurons from rodent and human fetal ventral mesencephalic (hfVM) donor tissue, when grafted to the dopamine-depleted striatum of experimental animals can be therapeutically helpful (Steinbeck & Studer, 2015; Wianny & Vezoli, 2017). Some patients in open-label hfVM trials (Freeman, Olanow, et al., 1995; Lindvall et al., 1990) exhibited clinical improvement. However, randomized double blinded, placebo-controlled, clinical trials indicated that these benefits were too variable to meet the trials' primary endpoints, although predefined secondary endpoints (Unified Parkinson's Disease Rating Scale, UPDRS) showed statistically significant benefits in younger (<60 years of age; (Freed et al., 2001)) or less impaired (UPDRS in off <49; (Olanow et al., 2003)) subjects. Additionally, some patients developed graft-induced dyskinesias (GID) (Freed et al., 2001; Hagell et al., 2002; Ma et al., 2002), possibly related to pre-existing L-DOPA-induced dyskinesias and the transplants containing serotonergic cells alongside the desired dopaminergic neurons (Hagell & Cenci, 2005; Lane & Smith, 2010). These findings prompted a reevaluation of the approach. More recently, the European collaborative consortium, TRANSEURO, revisited fetal transplantation in an open-label trial (NCT01898390) with 11 patients at relatively early disease stages who had not developed L-DOPA-induced dyskinesias prior to grafting (Barker & consortium, 2019).

Since no current therapy arrests or reverses the disease process, there is a major unmet need for new and effective PD treatments. Due to limited donor tissue availability and ethical problems in using fetal tissues, fVM therapies will likely not be useful for widespread clinical use, and therefore other cell sources such as embryonic stem cells (ESC) and induced pluripotent stem cells (iPSC) are being investigated. Clearly, there is a need for new and improved methods for generating dopaminergic neurons from pluripotent cells that may be used, e.g., to treat PD.

### SUMMARY OF THE INVENTION

In some aspects, the present disclosure overcomes limitations in the prior art by providing cultures of dopaminergic (DA) progenitor cells, preferably progenitor midbrain dopaminergic (mDA) cell cultures, that have improved therapeutic properties for the treatment of a disease or engraftment into a mammalian subject. Methods for making such DA progenitor cell cultures from pluripotent stem cells, such as induced pluripotent stem (iPS) cells, are provided. In contrast to previous experiments with mono-SMAD methodologies described in WO2018/035214 where equivalent patterning for DA progenitors was observed at several timepoints out to day 37 of differentiation culture, the present disclosure is based in part on the discovery that DA progenitor cells utilized after about 360-456 hours, more preferably about 384-432 hours, of differentiation culture using the mono-SMAD methods can surprisingly display superior properties for therapeutic applications, such as treatment of Parkinson's disease (PD). As shown in the below examples, these cells differentiated under these conditions within these amounts of time were observed to display dramatic improvements in engraftment and innervation in vivo using the 6-OHDA athymic nude rat model of PD. These ranges of time in the mono-SMAD differentiation methods as provided herein thus appear to be critical for generating cultures of cells that possess dramatically improved therapeutic potential. Behavioral experiments on rats that received cells administered to the striatum resulted in improved treatment of PD symptoms and recovery in vivo. Cellular maturity on survival and efficacy of transplanted mDA progenitors, immature neurons, and post-mitotic neurons were tested in hemiparkinsonian rats. Midbrain DA progenitor cells were markedly superior to immature or mature neurons in terms of survival, innervation, and efficacy. Homotopic (intranigral) engraftment demonstrated that mDA progenitors had greater capacity than immature neurons to innervate forebrain structures over long distances. When progenitors were assessed across a wide dose range, a clear structural and functional dose-response was observed. Although the grafts were derived from iPSCs, no teratomas or marked cell proliferation was observed. These data support the use of the human iPSC-derived mDA progenitors for transplantation to treat PD. Methods of treating a brain disease, such as PD, using the cell cultures or DA progenitors provided herein are also provided.

In some aspects, cryopreserved single-cell suspensions containing iPSC derived midbrain DA neuron progenitor cells (e.g., “FCDI DAPC-1” or D17 cells) are provided. The DA progenitor cells may be generated using about 360-456 hours, more preferably about 384-432 hours, of differentiation in culture using the mono-SMAD methods provided herein. These cells can be derived from allogeneic human iPS cells or iPS cell lines via directed differentiation to obtain a population of DA neuron progenitor cells. As shown in the below examples, such DA neuron progenitor cells were observed to have phenotypic markers (e.g., FIG. 1 and FIG. 2) and developmental potential similar to dopamine neurons precursors found in the substantia nigra region of the developing midbrain (e.g., FIG. 3, FIG. 13, and FIG. 14). FCDI DAPC-1 was observed to lack significant numbers of forebrain neurons and residual iPSCs that could be detrimental to therapeutic use (e.g., FIG. 5, FIG. 6, and Table 3). Unlike other DA cells that have been considered for therapeutic use, FCDI DAPC-1 is a proliferating neural progenitor cell population as demonstrated by EdU incorporation (FIG. 7). FCDI DAPC-1 displayed superior engraftment and innervation, which are characteristics associated with improved recovery in the 6-OHDA athymic rat model of PD (FIG. 9, FIG. 10, and FIG. 14).

In some aspects, dopaminergic neuronal precursor cells (e.g., FCDI DAPC-1) can be produced by culturing pluripotent stem cells, such as iPS cells, using mono-SMAD methodologies, wherein the cells are cultured under differentiation conditions for about 360-456 hours, or more preferably for about 384-432 hours. Mono-SMAD differentiation methodologies (also referred to as “mono-SMAD inhibition” or “mono-SMADi” methods) are described, e.g., in WO2018/035214, which is incorporated by reference herein in its entirety. Mono-SMADi methods can provide advantages over methods which require inhibition of SMAD signaling using two or more SMAD inhibitors. Generally, mono-SMAD methods involve use of only one SMAD inhibitor (i.e., a single SMAD inhibitor, and not a second-SMAD inhibitor). The mono-SMAD methods may include: (i) staggering the addition of a Wnt agonist (e.g., CHIR99021) to day 2 or day 3, (ii) re-optimizing the CHIR concentration (e.g., using from about 0.5-3.0 μM, 0.7-3 μM, 1-2.5 μM, 1.25-2.25 μM, from greater than about 1.25 μM to about 2 μM, or about 1.55, 1.65, 1.75 μM, or any range derivable therein), and/or (iii) including a MEK inhibitor (e.g., PD0325901) in the differentiation media on days 3-5. The methods may include, e.g., aspects (i and ii), (ii and iii), (i and iii), or (i, ii, and iii) above. In some embodiments, cells are exposed to a BMP inhibitor (e.g., dorsomorphin or LDN-193189), but the cells are not exposed to a TGF-β inhibitor such as SB431542. Cells can be differentiated of cells into midbrain DA neurons or FOXA2⁺/LMXIA⁺ cells. These methods may be used for mDA progenitor formation from iPS cell lines with media only including a single SMAD inhibitor (e.g., dorsomorphin only or LDN-193189 only).

An aspect of the present disclosure relates to a culture comprising midbrain dopaminergic (mDA) neuronal precursor cells generated by culturing human pluripotent cells in the presence of the following signaling modulators: (a) a first inhibitor of Small Mothers Against Decapentaplegic (SMAD) signaling, (b) at least one activator of Sonic hedgehog (SHH) signaling, and (c) at least one activator of wingless (Wnt) signaling; wherein the method does not comprise culturing the human pluripotent cells in the presence of a second inhibitor of Small Mothers Against Decapentaplegic (SMAD) signaling; and wherein the human pluripotent cells are cultured under conditions to induce differentiation for from about 360 to about 456 hours and then refrigerating or cryopreserving the cells; and wherein the midbrain dopaminergic precursor cells express both forkhead box protein A2 (FOXA2) and LIM homeobox transcription factor 1 (LMX1) (FOXA2⁺/LMX1⁺ cells). In some embodiments, the human pluripotent cells are cultured under conditions to induce differentiation for from about 384 to about 432 hours. In some embodiments, the mDA neuronal precursor cells do not express NURR1. The mDA neuronal precursor cells may express forkhead box protein A2 (FOXA2), LIM homeobox transcription factor 1 (LMX1), and EN1. The mDA neuronal precursor cells may further express OTX2. In some embodiments, forkhead box protein A2 (FOXA2) and LIM homeobox transcription factor 1 (LMX1) are co-expressed by from about 60% to about 100% or from about 85% to about 95% or more of the mDA neuronal precursor cells. In some embodiments, about 65-75% of the mDA neuronal precursor co-express both FOXA2 and LMX1. In some embodiments, the midbrain dopaminergic precursor cells express (FOXA2, LMX1A, ETV5, and EN1) and the midbrain dopaminergic precursor cells do not express (NURR1, TH, CALB1, BARHL1, or GRIK2). In some embodiments, the mDA neuronal precursor cells comprise proliferating or dividing cells. In some embodiments, at least about 40% or more, or about 50-75% of the mDA neuronal precursor cells are proliferating or dividing. The culture may further comprise about 5% or less of serotonergic neuronal precursor cells. The serotonergic neuronal precursor cells may express BARLH1. The culture may further comprises glial progenitor cells. The glial progenitor cells may express GLAST, SLC13A, CD44, and/or hGFAP. The inhibitor of SMAD signaling may be a BMP inhibitor, such as for example LDN-193189, dorsomorphin, DMH-1, or noggin. In some embodiments, the BMP inhibitor is LDN-193189. The LDN-193189 may be present at a concentration of from about 0.2 μM to about 4 μM, more preferably from about 1 μM to about 4 μM, from about 1 μM to about 3 μM, from about 0.5 μM to about 4 μM, from about 0.5 μM to about 2 μM, from about 0.2 μM to about 4 μM, from about 0.2 μM to about 2 μM, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 μM, or any range derivable therein. In some embodiments, the SMAD signaling inhibitor is a TGFβ inhibitor. The TGFβ inhibitor may be SB431542. In some embodiments, the SB431542 is present at a concentration of about 1-20 μM, about 5-15 μM, about 9-11 μM, or about 10 μM. The pluripotent cells may be cultured with the inhibitor of SMAD on culture days 1-15, 1-16, or 1-17. The pluripotent cells may be cultured with the inhibitor of SMAD on culture days 1-17. The pluripotent cells may be cultured with the inhibitor of SMAD substantially continuously or on a daily basis for 15, 16, or 17 days. The pluripotent cells may be cultured with the inhibitor of SMAD substantially continuously or on a daily basis for 17 days. The inhibitor of SMAD may be present at a concentration of about 50-2000 or 50-500 nM. The inhibitor of SMAD may be present at a concentration of about 180-240 nM. The method may further comprise contacting the pluripotent cells with a MEK inhibitor. In some embodiments, the MEK inhibitor is PD0325901. The PD0325901 may be present at a concentration of about 0.25-2.5 μM. The MEK inhibitor may be contacted to the pluripotent cells for about 1-3 days, or on days 1-3, 2-4, 3-5, or on days 1, 2, 3, 4, or 5, after initiation of contact with the inhibitor of SMAD signaling. In some embodiments, the MEK inhibitor is contacted to the pluripotent cells from about 24 to about 48 hours after initiation of contact with the inhibitor of SMAD signaling. In some embodiments, the MEK inhibitor is contacted to the pluripotent cells on a daily or substantially continual basis for about 3-4 days beginning about 1-2 days after initiation of contact with the inhibitor of SMAD signaling. In some embodiments, the MEK inhibitor is contacted to the pluripotent cells on days 2-5 or days 3-6 after initiation of contact with the inhibitor of SMAD signaling on day 1. The activator of Wnt signaling may be a GSK3 inhibitor. In some embodiments, the GSK3 inhibitor is CHIR99021. The CHIR99021 may be present at a concentration of about 1.5-2 μM, about 1.5-1.7 μM, about 1.6-1.7 UM or about 1.65 μM. In some embodiments, the CHIR99021 is present at a concentration of about 4-7 μM on days 9-17 after initiation of contact with the inhibitor of SMAD signaling. The activator of Wnt signaling may be contacted to the pluripotent cells 1-3 days after initiation of contact with the inhibitor of SMAD signaling. The activator of Wnt signaling may be contacted to the pluripotent cells within 24-48 hours after initiation of contact with the inhibitor of SMAD signaling. In some embodiments, the pluripotent cells are cultured with the activator of Wnt signaling substantially continuously or on a daily basis for 14, 15, or about 16 days. In some embodiments, the activator of Wnt signaling is contacted to the pluripotent cells on days 2-17 after initiation of contact with the inhibitor of SMAD signaling. In some embodiments, the activator of SHH signaling is purmorphamine or C25II Shh. The method may further comprises contacting the pluripotent cells with two activators of SHH signaling. The two activators of SHH signaling may be purmorphamine and C25II Shh. In some embodiments, the at least one activator of SHH signaling is contacted to the pluripotent cells on the same day as initiation of contact with the inhibitor of SMAD signaling or within 24-48 hours after initiation of contact with the inhibitor of SMAD signaling. In some embodiments, the at least one activator of SHH signaling is contacted to the pluripotent cells on days 1-7 with or after initiation of contact with the inhibitor of SMAD signaling. The method may further comprise contacting the pluripotent cells with FGF-8. In some embodiments, the FGF-8 is not contacted to the pluripotent cells on the same day as the initiation of contact with the inhibitor of SMAD signaling. In some embodiments, the FGF-8 is contacted with the pluripotent cells on days 9-17 or 11-17 after initiation of contact with the inhibitor of SMAD signaling. The FGF-8 may be present at a concentration of about 50-200 ng/mL. The pluripotent cells may comprise an antibiotic resistance transgene under the control of a neuronal promoter. The method may further comprises selecting for neural cells, midbrain DA neurons, or mDA neuronal precursor cells derived from the pluripotent cells by contacting cells with an antibiotic, a chemotherapeutic, a DNA crosslinker, a DNA synthesis inhibitors, or a mitotic inhibitor. The method may further comprise contacting the pluripotent cells with an antibiotic or a chemotherapeutic (e.g., mitomycin C). In some embodiments, the mitomycin C is contacted with the pluripotent cells on days 27, 28, 29, and/or 30 after initiation of contact with the inhibitor of SMAD signaling. In some embodiments, the antibiotic is G418 (geneticin). The method may further comprise culturing or incubating the pluripotent cells in a media comprising a ROCK inhibitor prior to initiation of contact with the inhibitor of SMAD signaling. The method may further comprise contacting the pluripotent cells with blebbistatin. The blebbistatin may be contacted with the cells on day 5 and day 17 of differentiation. In some embodiments, the mDA dopaminergic precursor cells do not express NURR1, MAP2, or TH. The mDA dopaminergic precursor cells may nonetheless retain the potential to express NURR1, MAP2, and/or TH, e.g., in the future after additional growth or differentiation. In some embodiments, the mDA dopaminergic precursor cells express EN1. The mDA dopaminergic precursor cells may express low levels of or substantially no PITX2 or PITX3, although both of these markers have been observed in mature dopaminergic neurons. In some embodiments, the mDA dopaminergic precursor cells express GBX2, OTX1, OTX2, ETV5, CORIN, and/or DCX. In some embodiments, the pluripotent cells are human induced pluripotent stem (iPS) cells. The LMX1 may be LIM homeobox transcription factor 1 alpha (LMX1A). In some embodiments, about 5% or less (e.g., less than about 1%, or less than 0.5%), of the cells in the cell composition are serotonergic cells or serotonergic precursor cells. The method may further comprises incubating the human pluripotent cells in the presence of a DNase or an endonuclease (e.g., DNase I or Benzonase®). The DNase I or Benzonase® may be present at a concentration of about 10-20 U/mL or about 10-15 U/mL. For example, the DNase I Benzonase® may by added on day 17 of culture, e.g., to reduce cell clumping in cell preparations such as single cell preparations. In some embodiments, the human pluripotent cells are cultured in the presence of an endonuclease on at least one of days 4-6 after initiation of contact with the inhibitor of SMAD signaling. The human pluripotent cells may be cultured in the presence of an endonuclease on day 5 after initiation of contact with the inhibitor of SMAD signaling. The culture may be comprised in a container means. In some embodiments, the midbrain dopaminergic neurons or midbrain dopaminergic neuronal precursor cells are comprised in a pharmaceutical preparation. The pharmaceutical preparation may be formulated for injection. In some embodiments, the pharmaceutical preparation comprises a hyaluronic acid matrix. The culture may comprise from about 2,500 cells/μL to about 150,000 cells/μL, from about 2,500 cells/μL to about 100,000 cells/μL, from about 10,000 cells/μL to about 150,000 cells/μL, from about 40,000 cells/μL to about 100,000 cells/μL, or about 15,000-45,000 cells/μL. The cells may be midbrain dopaminergic neuronal precursor cells or DAPC-1 cells. The culture may contain from about 1e6 to about 25e6, more preferably from about 3e6 to about 9e6 cells. In some embodiments, about 10% or less, more preferably about 7% or less of the cells in the culture are serotonergic precursor cells. In some embodiments, about 5% or less of the cells in the culture are serotonergic precursor cells. In some embodiments, about 5% or less of the cells in the culture express SERT and TPH2. As shown in the below examples, cultures were observed to contain approximately 5% serotonergic cells (serotonergic precursor cells), based on expression of SERT and TPH2 at day 14, and the serotonergic neurons did not survive after engraftment. While in some preferred embodiments, the total number of serotonergic cells is about 5% or less, in some embodiments, the culture may contain about 6%, 7%, 8%, or higher of serotonergic cells. In some embodiments, about 0.1-5% or less of the cells in the culture express FOXG1, and/or wherein about 0.1-5% or less of the cells in the culture express PAX6. In some embodiments, less than about 1% of the cells in the culture express FOXG1, and/or wherein less than about 1% of the cells in the culture express PAX6.

Another aspect of the present disclosure relates to a method of treating a disease in a mammalian subject comprising administering to the subject a therapeutically effective amount of the culture described above or herein, e.g., preferably wherein the culture is administered to the brain of the subject. The mammalian subject may be a human. The disease may be a disease of the central nervous system (CNS). In some embodiments, the disease is Parkinson's disease (PD) or a Parkinson-plus syndrome (PPS). In some embodiments, the culture comprises mDA precursor cells that express Engrailed 1 (EN1), but do not express NURR1. In some embodiments, the culture comprises dopaminergic neurons that are not fully differentiated. The culture may be administered to the striatum, such as the putamen or substantia nigra, of the subject. In some embodiments, the culture is administered to more than one location into the striatum or putamen of the subject. The culture may be is administered at multiple sites and/or at multiple needle tracts into the striatum or putamen of the subject. The culture may be comprised in a pharmaceutical composition. The pharmaceutical composition may comprise a hyaluronic acid matrix. In some embodiments, the culture comprises about 15,000-45,000 cells/μL, or about 2e5, 2.5e5, 3e5, 4e5, 4.5e5, or any range derivable therein midbrain dopaminergic neuronal precursor cells. The culture may contain from about 1e6 to about 25e6, more preferably from about from about 3e6 to about 9e6 cells. The culture may comprises from about 2,500 cells/μL to about 150,000 cells/μL, from about 10,000 cells/μL to about 150,000 cells/μL, from about 40,000 cells/μL to about 100,000 cells/μL, or about 10000, 20000, 30000, 40000, 50000, 60000, 70000, 80000, 90000, 100000 cells/μL or any range derivable therein. In some embodiments, the subject has Parkinson's disease and wherein the subject exhibits improvement in at least one motor symptom after the administration of the culture. In some embodiments, the subject exhibits a reduction in one or more of tremor, muscle rigidity, slowness of movement, falls, dizziness, movement freezing, muscle cramps, or dystonia. The midbrain dopaminergic precursor cells may at least partially reinnervate the striatum or putamen of the subject. In some embodiments, the midbrain dopaminergic precursor cells exhibit limited, little or no proliferation after the administration. The midbrain dopaminergic precursor cells may nonetheless comprise at least some cells that are still dividing or proliferating, and the midbrain dopaminergic precursor cells may continue differentiating after the administration. In some embodiments, less than about 1%, or preferably less than 0.5%, of the cells in the cell culture are serotonergic cells. In some embodiments, at least 80% of administered cells differentiate into differentiated cells that express both FOXA2 and LMX1 after administration to the subject. In some embodiments, at least 85% of the differentiated cells express both FOXA2 and LMX1. In some embodiments, at least about 60% of the administered cells express both FOXA2 and LMX1. The culture may be cryogenically frozen (e.g., cryogenically frozen in liquid nitrogen) prior to the administering. For example, the cells may be cryogenically frozen for storage and subsequently brought to room temperature the cells are administered to the subject. The differentiated cells expressing FOXA2 and LMX1 may further express at least one marker selected from the group consisting of engrailed (EN1), tyrosine kinase (TH), orthodenticle homeobox 2 (OTX2), nuclear receptor related 1 protein (NURR1), Neuron-specific class III beta-tubulin (Tuj1), TTF3, paired-like homeodomain 3 (PITX3), achaete-scute complex (ASCL), early B-cell factor 1 (EBF-1), early B-cell factor 3 (EBF-3), transthyretin (TTR), synapsin, dopamine transporter (DAT), and G-protein coupled, inwardly rectifying potassium channel (Kir3.2/GIRK2), CD142, DCSM1, CD63 and CD99. In some embodiments, the differentiated cells expressing FOXA2 and LMX1, or FOXA2 and TH, further express engrailed, PITX3, and NURR1. In some embodiments, about 10-25% of the cells in the cell culture co-express FOXA2 and tyrosine hydroxylase (TH). The pluripotent cells may be human induced pluripotent stem (iPS) cells. In some embodiments, the LMX1 is LIM homeobox transcription factor 1 alpha (LMX1A). In some embodiments, less than about 5%, less than about 1%, or less than 0.5%, of the cells in the cell composition are serotonergic cells. In some embodiments, the administration does not result in host gliosis. The administration may result in no or essentially no growth or proliferation of non-neuronal cells in the brain of the subject. The administration may result in the engraftment of the mDA precursor cells in the brain of the subject and/or innervation of at least part of the brain of the subject by the mDA precursor cells. The administration may be via injection (e.g., stereotaxic injection).

An aspect of the present disclosure relates to an in vitro method for preparing a cell composition comprising human cells that express both forkhead box protein A2 (FOXA2) and LIM homeobox transcription factor 1 (LMX1) (FOXA2⁺/LMX1⁺ cells) comprising culturing human pluripotent cells in the presence of the following signaling modulators: (a) a first inhibitor of Small Mothers Against Decapentaplegic (SMAD) signaling, (b) at least one activator of Sonic hedgehog (SHH) signaling, and (c) at least one activator of wingless (Wnt) signaling; wherein the method does not comprise culturing the human pluripotent cells in the presence of a second inhibitor of Small Mothers Against Decapentaplegic (SMAD) signaling; and wherein the human pluripotent cells are cultured under conditions to induce differentiation for from about 360 to about 456 hours, or any range derivable therein, and then refrigerating or cryopreserving the cells. In some embodiments, the human pluripotent cells are cultured under conditions to induce differentiation for from about 384 to about 432 hours. In some embodiments, the human cells do not express NURR1. The human cells may express forkhead box protein A2 (FOXA2), LIM homeobox transcription factor 1 (LMX1), and Engrailed Homeobox 1 (EN1). The human cells may further express OTX2. In some embodiments, forkhead box protein A2 (FOXA2) is expressed by about 85-95% of the cells. In some embodiments, FOXA2 and LIM homeobox transcription factor 1 (LMX1) are co-expressed by from about 65% to about 85% or more, or from about 65% to about 75% of the human cells. The inhibitor of SMAD signaling may be a BMP inhibitor (e.g., LDN-193189, dorsomorphin, DMH-1, or noggin). In some embodiments, the BMP inhibitor is LDN-193189. The LDN-193189 may, for example, be present at a concentration of from about 0.2 μM to about 4 μM, or at a concentration of from about 1 μM to about 3 μM, from about 0.5 μM to about 4 μM, from about 0.5 μM to about 2 μM, from about 0.2 μM to about 4 μM, from about 0.2 μM to about 2 μM, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 μM, or any range derivable therein. In some embodiments, the SMAD signaling inhibitor is a TGFβ inhibitor (e.g., SB431542). The SB431542 may be present at a concentration of about 1-20 μM, 5-15 μM, 9-11 μM, or about 10 μM. The pluripotent cells may be cultured with the inhibitor of SMAD on culture days 1-15, 1-16, or 1-17. In some embodiments, the pluripotent cells are cultured with the inhibitor of SMAD on culture days 1-17. The pluripotent cells may be cultured with the inhibitor of SMAD substantially continuously or on a daily basis for 15, 16, or 17 days. In some embodiments, the pluripotent cells are cultured with the inhibitor of SMAD substantially continuously or on a daily basis for 17 days. The inhibitor of SMAD may be present at a concentration of about 50-2000 or about 50-500 nM. In some embodiments, the inhibitor of SMAD is present at a concentration of about 180-240 nM. The method may further comprise contacting the pluripotent cells with a MEK inhibitor (e.g., PD0325901). The PD0325901 may be present at a concentration of about 0.25-2.5 μM. In some embodiments, the MEK inhibitor is contacted to the pluripotent cells for about 1-3 days, or on days 1-3, 2-4, 3-5, or on days 1, 2, 3, 4, or 5, after initiation of contact with the inhibitor of SMAD signaling. In some embodiments, the MEK inhibitor is contacted to the pluripotent cells from about 24 to about 48 hours after initiation of contact with the inhibitor of SMAD signaling. In some embodiments, the MEK inhibitor is contacted to the pluripotent cells on a daily or substantially continual basis for about 3-4 days beginning about 1-2 days after initiation of contact with the inhibitor of SMAD signaling. In some embodiments, the MEK inhibitor is contacted to the pluripotent cells on days 2-5 or days 3-6 after initiation of contact with the inhibitor of SMAD signaling on day 1. The activator of Wnt signaling may be a GSK3 inhibitor (e.g., CHIR99021). In some embodiments, the CHIR99021 is present at a concentration of about 1.5-1.7 μM, about 1.6-1.7 μM, about 1.65 μM, or any range derivable therein. In some embodiments, the CHIR99021 is present at a concentration of about 4-7 μM on days 9-17 after initiation of contact with the inhibitor of SMAD signaling. In some embodiments, the activator of Wnt signaling is contacted to the pluripotent cells 1-3 days after initiation of contact with the inhibitor of SMAD signaling. The activator of Wnt signaling may be contacted to the pluripotent cells within 24-48 hours after initiation of contact with the inhibitor of SMAD signaling. In some embodiments, the pluripotent cells are cultured with the activator of Wnt signaling substantially continuously or on a daily basis for 14, 15, or about 16 days. In some embodiments, the activator of Wnt signaling is contacted to the pluripotent cells on days 2-17 after initiation of contact with the inhibitor of SMAD signaling. The activator of SHH signaling may be purmorphamine or C25II Shh. The method may further comprise contacting the pluripotent cells with two activators of SHH signaling (e.g., purmorphamine and C25II Shh). In some embodiments, the at least one activator of SHH signaling is contacted to the pluripotent cells on the same day as initiation of contact with the inhibitor of SMAD signaling or within 24-48 hours after initiation of contact with the inhibitor of SMAD signaling. The at least one activator of SHH signaling may be contacted to the pluripotent cells on days 1-7 with or after initiation of contact with the inhibitor of SMAD signaling. The method may further comprises contacting the pluripotent cells with FGF-8. In some embodiments, the FGF-8 is not contacted to the pluripotent cells on the same day as the initiation of contact with the inhibitor of SMAD signaling. In some embodiments, the FGF-8 is contacted with the pluripotent cells on days 9-17 or 11-17 after initiation of contact with the inhibitor of SMAD signaling. The FGF-8 may be present at a concentration of about 50-200 ng/mL. The pluripotent cells may comprise an antibiotic resistance transgene under the control of a neuronal promoter. The method may further comprise selecting for neural cells, midbrain DA neurons, or mDA precursor cells derived from the pluripotent cells by contacting cells with an antibiotic, a chemotherapeutic, a DNA crosslinker, a DNA synthesis inhibitors, or a mitotic inhibitor. The method may further comprise contacting the pluripotent cells with an antibiotic or a chemotherapeutic. The chemotherapeutic may be mitomycin C. In some embodiments, the mitomycin C is contacted with the pluripotent cells on days 27, 28, 28, and/or 29 after initiation of contact with the inhibitor of SMAD signaling. In some embodiments, the antibiotic is G418 (geneticin). The method may further comprise culturing or incubating the pluripotent cells in a media comprising a ROCK inhibitor prior to initiation of contact with the inhibitor of SMAD signaling. The method may further comprise contacting the pluripotent cells with blebbistatin. In some embodiments, the blebbistatin is contacted with the cells on day 5 and day 17 of differentiation. In some embodiments, at least 40%, at least 60%, at least 80%, or at least 85% of the human pluripotent cells differentiate and express both FOXA2 and LMX1. In some embodiments, about 10-25% of the human pluripotent cells differentiate and express both FOXA2 and tyrosine hydroxylase (TH). The pluripotent cells may be human induced pluripotent stem (iPS) cells. In some embodiments, the LMX1 is LIM homeobox transcription factor 1 alpha (LMX1A). In some embodiments, the differentiated cells expressing FOXA2 and LMX1, or FOXA2 and TH, further express at least one marker selected from the group consisting of orthodenticle homeobox 2 (OTX2), nuclear receptor related 1 protein (NURR1), Neuron-specific class III beta-tubulin (Tuj1), TTF3, paired-like homeodomain 3 (PITX3), achaete-scute complex (ASCL), early B-cell factor 1 (EBF-1), early B-cell factor 3 (EBF-3), transthyretin (TTR), synapsin, dopamine transporter (DAT), and G-protein coupled, inwardly rectifying potassium channel (Kir3.2/GIRK2), CD142, DCSM1, CD63 and CD99. The FOXA2⁺/LMX1⁺ cells may further express engrailed EN1. The FOXA2⁺/LMX1⁺ cells may further express EN1, Pax8, and ETV5. In some embodiments, the FOXA2⁺/LMX1⁺ cells do not express NURR1. The FOXA2⁺/LMX1⁺ cells may express GBX2, OTX1, OTX2, ETV5, CORIN, and/or DCX. In some embodiments, less than about 1%, preferably less than 0.5%, of the cells in the cell composition are serotonergic cells. The method may further comprise incubating human pluripotent cells in the presence of a DNase or an endonuclease. The endonuclease may be DNase I or Benzonase®. The DNase I or Benzonase® may be present at a concentration of about 10-20 U/mL or at a concentration of about 10-15 U/mL, or any range derivable therein. In some embodiments, the human pluripotent cells are cultured in the presence of an endonuclease on at least one of days 4-6 after initiation of contact with the inhibitor of SMAD signaling. In some embodiments, the human pluripotent cells are cultured in the presence of an endonuclease on day 5 after initiation of contact with the inhibitor of SMAD signaling.

In some embodiments, cells differentiated for longer or shorter periods of time than stated above using the mono-SMAD methods provided herein ranges are provided. For example, in addition to D17 cells, cells at a later stage of differentiation, such as D24 cells and/or D37 cells are provided herein and can be administered to a subject to treat a neurological or brain disease. In some embodiments, cultures comprising cells that have been differentiated using the mono-SMAD methodologies provided herein for at least 18, 19, 20, 21, 22, 23, 24, 25, 26, 27, 28, 29, 30, 31, 32, 33, 34, 35, 36, or 37 days, or any range derivable therein, are provided and can, e.g., be included in a pharmaceutical composition or used for in vitro testing (e.g., toxicology testing, drug screening, electrophysiological testing, etc.) or used to treat a neurological disease in vivo. In some embodiments, cells are provided herein that are differentiated using the mono-SMAD methodologies provided herein and for a period of time of about 12, 13, 14, 15, 16 days, or any range derivable therein, and it is anticipated that such cells may be administered to a mammalian subject to treat a neurological disease as described herein such as, e.g., PD. As shown in the below examples, D17, D24, and D37 cells may express the following cellular markers, as follows:

TABLE X1

| Marker | D17 | D24 | D37 |
|---|---|---|---|
| FoxA2 | + | + | + |
| LMX1A | + | + | + |
| NURR1 | − | + | + |
| TH | − | + | + |
| CALB1 | − | − | + |
| ETV5 | + | + | + |
| EN1 | + | − | − |
| BARHL1 | − | − | − |
| GIRK2 | − | − | + |

“+” = Expression observed;
“−” = very low expression or expression not observed

Another aspect of the present disclosure relates to a culture comprising midbrain dopaminergic neurons or midbrain dopaminergic neuronal precursor cells generated by the method described above or herein. The culture may be comprised in a container means. In some embodiments, the midbrain dopaminergic neurons or midbrain dopaminergic neuronal precursor cells are comprised in a pharmaceutical preparation. The pharmaceutical preparation may be formulated for injection.

Another aspect of the present disclosure relates to a method of screening a test compound comprising: (a) contacting FOXA2⁺/LMX1A+ cells differentiated by the methods described above or herein or the mDA precursor cells (e.g., D17 cells) described above or herein with the test compound, and (b) measuring the function, physiology, or viability of the cells. The measuring may comprise testing for a toxicological response or an altered electrophysiological responses of the cells. In some embodiments, the cells are midbrain dopaminergic neurons or midbrain dopaminergic neuronal precursor cells.

Additional conditions and methods that may be used in combination with the present invention may be found, e.g., in U.S. 2015/0265652, U.S. 2015/0010514, and WO2013/067362, which are incorporated by reference herein in their entirety. Additional methods for purifying or promoting differentiation of pluripotent cells into neuronal or midbrain DA neurons that may be used in combination with the present invention include, e.g., Kirkeby et al. (2012), Kriks, et al. (2011); Chung, et al. (2011), Xi et al. (2012); Young et al. (2014); Jaeger et al. (2011), Jiang et al. (2012), and US2016/0177260.

As used herein, the “differentiation day” refers to the day of incubation of cells in a media, wherein initiation of exposure of pluripotent cells to a differentiation media on day 1. In some preferred embodiments, the differentiation media on day 1 includes a single SMAD inhibitor. Prior to incubation or culture in a differentiation media, cells may be incubated, e.g., for 1, 2, or 3 days prior to incubation in the differentiation media (i.e., on day 0, day −1, and/or day −2) in a medium comprising or consisting of Essential 8™ Basal Medium and Essential 8™ Supplement (Thermo Fisher Scientific; Waltham, MA), optionally with the addition of a ROCK inhibitor (e.g., inclusion of about 0.25-5 μM, 0.5, 0.75, 1, 1.25, 1.5, 2, 3, 4, or any range derivable therein of H1152, e.g., on day −2), and/or blebbestatin (e.g., at a concentration of about 0.1-20 μM, more preferably about 1.25-5 μM, or about 2.5 μM).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1: FOXA2 flow cytometry of final product.

FIG. 2: DA Progenitor Purity (FOXA2+/LMX1+).

FIG. 3: DA Neuron Differentiation Potential (NURR1+/S100β⁻).

FIG. 4: Neuron Differentiation Potential (MAP2+/Nestin−).

FIGS. 5A-B: Forebrain Neurons. FCDI DAPC-1 cells were stained with (FIG. 5A) anti-PAX6 (Biolegend #901301) or (FIG. 5B) anti-FOXG1. iCell GABA Neurons (FCDI) are shown as a positive control; they are cells patterned to a forebrain phenotype, predominantly GABAergic, and contain a subpopulation of PAX6+ neurons and also FOXG1+ neurons.

FIG. 6: RT-QPCR for Residual iPSCs.

FIGS. 7A-C: FCDI DAPC-1 consists of proliferating cells. (FIG. 7A) Time course of EdU incorporation throughout FCDI DAPC-1 manufacturing. Nearly half of the FCDI DAPC-1 cells are proliferating. (FIG. 7B) Timecourse of EdU incorporation in the FOXA2+ population of FCDI DAPC-1 in post thaw culture. Proliferation decreases as cells differentiate from the mDA progenitor stage to mature DA neurons. (FIG. 7C) EdU that was incorporated into mDA progenitor cells at day 17 for a period of 24 hours is retained in Nurr1+ cells 12 days after maturation of progenitor cells.

FIG. 8: Amphetamine Rotations in Nude Rats. Rotations shown are the mean of all recordings taken. Error bars represent the standard error of the mean, n=10-12 animals per group.

FIG. 9: Striatal Re-Innervation 6 months post-transplant. Re-innervation shown using staining for TH positive cells. Although no statistical difference between D17 and D24 in the number of TH+ cells in the graft, the innervation of the striatum by the D17 cells was observed to be better than the D24 cells.

FIG. 10: Intranigral grafts Innervate the Striatum. When cells are injected directly into the subtantia nigra, the D17 grafts showed better innervation into the medial forebrain bundle and striatum compared to the D24 cells.

FIG. 11: qPCR Progenitor Marker Time course. Progenitor markers vary slightly between D17 and D24 cells. Lmx1, Pitx2, Nurr1, and Pitx3 are expressed at a higher level in D24 cells whereas En-1, Pax8, ETV5, and Glast are expressed at higher levels in the D17 cell.

FIG. 12: qPCR Markers Time Course. Mature markers also varied in expression; AQP4 and tyrosine hydroxylase (TH) are expressed at higher levels in D24 compared to D17 cells.

FIG. 13: Immunocytochemistry (ICC) comparison of D17 and D24 cultures.

FIG. 14: Violin Plots of Gene Expression.

FIG. 15: Amphetamine Rotations using alternative cell types. Rotations shown are the mean of all recordings taken. Error bars represent the standard error of the mean n=4-10 animals.

FIGS. 16A-C: Cell population percentages. Percent hNuc was calculated by dividing the number of hNuc+ cells by 450,000 injected cells, TH, and Ki67 are percentages of engrafted hNuc in same graft. Results are shown for hNuc (FIG. 16A), TH (FIG. 16B), and Ki67 (FIG. 16C). Data from tissue slices from rats are shown. The percentage of each population is listed in the title of each graph (hNuc from total input, TH from total hNuc counted, and Ki67 from total hNuc counted).

FIGS. 17A-C: Stereology Analysis for hNuc, TH, and Ki67. Every 12th section (½ series) was stained for hNuclei, TH, or hKi67 and quantified by unbiased stereology. For each animal, the graft area was outlined and counted. Each graph has a unique Y-axis. FIG. 17A, the number of hNuc positive cells from each animal in each test group, the mean and standard error of the mean (SEM), are shown. FIG. 17B, the number of TH positive cells from each animal in each group, including the mean and SEM, are shown. FIG. 17C, the number of Ki67 positive cells from each animal in each group, including the mean and SEM, are shown.

FIGS. 18A-C: Panel 1 (top) shows FoxA2 expression by flow cytometry in cells made with 1.50 μM CHIR (FIG. 18A), 1.75 μM CHIR (FIG. 18B), and 2.00 μM CHIR (FIG. 18C). Panel 2 (bottom) shows FoxA2 (y-axis)/Lmx (x-axis) expression by flow cytometry.

FIG. 19: Expression of genes in cells generated after varying days of differentiation, measured using qPCR.

FIGS. 20A-J: Characterization and analysis of function, survival, and innervation of D17 progenitors in vivo. Time-based analysis of (FIG. 20A) d-amphetamine-induced rotations measured pre-operatively and at 2, 4, and 6 months post-engraftment. (FIG. 20B) Stereological estimates of hNuclei-ir cells contained in grafts of low, medium, high, or maximum feasible dose. Quantification of (FIG. 20C) stereological estimates of TH-ir cells and (FIG. 20D) stereological estimates for each group. (E) Representative images of graft sections stained for hGFAP (scale bar 200*m) and (F) 5-HT (scale bar 1 mm (inset 25*m)). Representative images containing grafts of low, medium, high, and maximum feasible dose for DAB-processed (FIG. 20G) hNuclei and (FIG. 20H) TH or immunofluorescent triple-labeled (FIG. 20I) hNuclei/TH/FoxA2 (green/red/blue) and (FIG. 20J) TH/Girk2/Calbindin (green/red/blue). Scale bar=500*m.

FIGS. 21A-B: Differentiation and gene expression in vitro. (FIG. 21A) Schematic representation of differentiation and transplantation. MMC=mitomycin c. (FIG. 21B) qPCR comparing mRNA expression at iPSC-mDA differentiation Days 17, 24, and 37 of target and off-target regional, cell type, and neural maturation markers. Three biological replicates were analyzed in technical triplicate for each process timepoint. Mean Ct values are expressed as relative to glyceraldehyde-3-phosphate dehydrogenase (GAPDH) (ΔCt). Error bars are SEM. Significance indicated in Table 7.

FIGS. 22A-B Protein expression in vitro. (FIG. 22A) Flow cytometry comparing immunoreactive populations at iPSC-mDA differentiation Days 17, 24, and 37 of mDA target markers. Quantification of positive cell populations of live cells shown for FOXA2+, FOXA2+/LMX1+, NURR1+, MAP2+ and FOXA2+/TH+. Three biological replicates were analyzed for each time point (Mean±SEM). (FIG. 22B) Immunocytochemistry comparing immunoreactive populations at iPSC-mDA differentiation Days 17, 24, and 37 of mDA target and off-target markers. Images are representative of three biological replicates analyzed for each time point.

FIGS. 23A-E: Graft survival and function. Time-based analysis of (FIG. 23A) d-amphetamine-induced rotations measured pre-operatively and at 2, 4, and 6 months post-engraftment. At 4 months post-transplantation, P<0.0005 for D17 and P<0.005 for G418; at 6 months post-transplantation, P<0.0005 for D17 and D24 and P<0.05 for G418. Data were analyzed by mixed ANOVA with Tukey's adjustment; error bars are SEM. Comparisons were made to vehicle group. Representative graft sections stained for (FIG. 23B) hNuclei and (FIG. 23C) hKi-67 with graft borders indicated by black outline. Quantification by unbiased stereology of (FIG. 23D) hNuclei-ir (P<0.0001 D17 vs. D37/G418; P<0.0005 and P<0.005 for D24 vs. D37/G418, respectively) and (FIG. 23E) hKi-67-ir cells (P<0.05 for D17 vs. D37; P<0.01 for D17 vs. G418; P<0.05 for D24 vs. D37). Scale bar=500 μM in (FIG. 23B); 50 μM in (FIG. 23C, inset). hNuclei estimates were analyzed by one-way ANOVA with Tukey's adjustment; error bars represent SD. hKi-67 estimates were analyzed by Kruskal-Wallis test and Dwass-Steele-Critchlow-Fligner post-hoc.

FIGS. 24A-D: Visualization of dopaminergic phenotype in vivo. Representative graft-containing sections stained for (FIG. 24A) DAB-processed TH. Quantification of (FIG. 24B) TH-ir cells contained within grafts after 6 months in vivo (P<0.0001 and P<0.005 for D17 vs. D37/G418; P<0.0005 and P<0.01 for D24 vs. D37/G418, respectively). (FIG. 24C) Optical density of graft-derived TH-ir fibers. Significant P-values were calculated for D17 vs D24, D37, and G418 (P<0.0005, P<0.0001, P<0.05); D24 vs D37 (P<0.001); and G418 vs D37 (P<0.0005). (FIG. 24D) Immunofluorescently triple-labeled for TH/FOXA2/hNuclei (green/red/blue). Scale bar (A)=500 μM; (D)=20 μM.

FIG. 25: Long-range innervation of grafted cells transplanted in substantia nigra. Representative computer-inverted micrographs of hNCAM immunoreactivity in coronal sections spanning from the forebrain to the site of transplant in the substantia nigra. DAB-processed images were inverted and adjusted to show extent of innervation; all enhancements were applied to each sample in an identical fashion. AC=anterior commissure, AON=anterior olfactory nucleus, cc=corpus callosum, CPu=caudate/putamen, Fr=frontal cortex, NAc=nucleus accumbens, PrL=prelimbic area, Sept=Septum, T=transplant, Tu=olfactory tubercle.

FIGS. 26A-F: Quantitative analysis of function, survival, and innervation of D17 progenitors in vivo. Time-based analysis of (FIG. 26A) d-amphetamine-induced rotations measured pre-operatively and at 2, 4, and 6 months post-engraftment. At 4 months post-transplantation, P<0.0001 for MFD and P<0.0005 for high dose; at 6 months post-transplantation, P<0.0001 for MFD and high dose; P<0.005 for medium dose; analyzed by mixed ANOVA with Tukey's adjustment. Comparisons were made to vehicle group. (FIG. 26B) Stereological estimates of hNuclei-ir cells (visualized in FIG. 26E) contained in grafts of low, medium, high, or ‘maximum feasible’ dose. P<0.0001 for all comparisons by one-way ANOVA with Tukey's adjustment. Stereological estimates of (FIG. 26C) TH-ir cells (visualized in FIG. 26F) contained in grafts of low, medium, high, or ‘maximum feasible; dose. P<0.0001 for MFD vs. all groups; P<0.005 and P<0.05 for high dose vs. medium and low dose groups, respectively; analyzed by one-way ANOVA with Tukey's adjustment. Quantification of (FIG. 26D) graft-derived TH optical density. One-way ANOVA with Tukey's adjustment showed P<0.0001 for MFD vs medium and low doses and high vs. medium and low doses; P<0.05 for MFD vs. high dose. One-way or mixed effects ANOVA with Tukey's adjustment for histological or behavioral data, respectively; error bars represent SD or SEM for histological or behavioral data, respectively. Images for low dose group are from a rat with substantial surviving graft. Scale bar=500 μM.

FIGS. 27A-C: Correlations of dopaminergic phenotype with behavioral recovery and visualization of mDA subtype. (FIG. 27A) Estimated number of TH-ir cells and TH optical densitometric measurements plotted against the absolute value of the magnitude of change in net d-amphetamine-induced rotations and fitted with logarithmic regression curve. Linear regression for low/medium or high/‘maximum feasible’ doses and behavioral recovery. Representative images containing grafts of low, medium, high, and ‘maximum feasible’ dose for immunofluorescent triple-labeled (FIG. 27B) hNuclei/TH/FOXA2 (blue/green/red) and (FIG. 27C) TH/GIRK2/Calbindin (green/red/blue).

FIGS. 28A-E: Non-dopaminergic cell types observed in grafts. Representative (FIG. 28A) micrographs of graft sections stained for hKi-67 and (FIG. 28B) stereological estimates for each group. (FIG. 28C) Representative images of graft sections stained for hGFAP (glia), (FIG. 28D), Iba1 (microglia), and (FIG. 28E) 5-HT (serotonergic neurons). Scale bar (FIG. 28A)=100 μM; (FIG. 28C)=200 μM; (FIG. 28D)=500 μM; (FIG. 28E)=1 mm (E, inset)=25 μM. P<0.05 for medium vs. low dose; P<0.005 for all other comparisons by Kruskal-Wallis test with Dwass, Steel, Critchlow-Fligner method.

FIG. 29: Visualization of protein expression in vitro. Immunocytochemistry comparing immunoreactive populations at iPSC-mDA differentiation Days 17, 24, and 37 of mDA target and off-target markers. Images are representative of three biological replicates analyzed for each timepoint.

FIGS. 30A-B: Short-term engraftment. Coronal sections containing bilateral G418, D37, D24, or D17 striatal grafts in intact rats 3 months post-injection stained for (FIG. 30A) hNCAM or (FIG. 30B) TH.

FIGS. 31A-I: Single cell gene expression in vitro. Single cell qPCR (Fluidigm) comparing mRNA expression at iPSC-mDA differentiation Days 17, 24, and 36 of target markers for A) FoxA2, B) LMX1A, C) NURR1, D) TH, E) CALB1, F) ETV5, G) EN1, H) BARHL1, and I) GIRK2. 96 individual cells were evaluated for each process timepoint. Log 2 expression values for each cell represented as a single mark on the graph. Error bars are SEM.

FIG. 32: FCDI DAPC-1 flow cytometry assays for potentially dangerous non-target cell markers FOXG1⁺ and PAX6⁺ cells demonstrate a very low percentage of forebrain neuron progenitors.

FIG. 33: FCDI DAPC-1 qPCR assay for serotonergic cell population from 0-19DPT.

FIG. 34: FCDI DAPC-1 qPCR assay for SERT at 14DPT shows consistently low expression across batches.

FIG. 35: FCDI DAPC-1 ICC assay for serotonergic marker, 5-HT supports qPCR results for SERT and TPH2. Representative images are shown for ICC stain of 5-HT (red) for timepoints 1-, 8-, 15-, and 20-DPT.

### DESCRIPTION OF ILLUSTRATIVE EMBODIMENTS

In some aspects, the present invention overcomes limitations in the prior art by providing compositions and methods for differentiating pluripotent cells, such as induced pluripotent stem cells, into dopaminergic (DA) neuronal precursor cells that can display significantly improved properties for treatment of brain diseases in vivo. The methods may involve differentiating the pluripotent cells in the presence of a single SMAD inhibitor (“mono-SMAD inhibition”) for specific amounts of time, such as about 360-456 hours, or more preferably about 384-432 hours, under the mono-SMAD conditions. Generally, and in contrast to previous dual-SMAD methods, mono-SMAD methods involve use of only one SMAD inhibitor, in contrast to dual-SMAD methods that utilize two SMAD inhibitors. In contrast to previous studies that concluded that immature neurons expressing NURR1 are more efficacious than less mature progenitors that do not express NURR1 (Ganat et al., 2012; Qiu et al., 2017), midbrain dopaminergic (mDA) precursor cells are provided herein (e.g., D17 cells) that do not express NURR1 and have displayed superior efficacy in vivo (e.g., for treatment of PD) as compared to mDA precursor cells that express NURR1. As shown in the below examples, cell cultures comprising midbrain DA neuronal precursor cells differentiated for these specific amounts of time were surprisingly observed to display superior properties in vivo, as compared to cell cultures differentiated for other periods of time using these mono-SMAD methods, and significant improvements in engraftment and innervation were observed using these cell cultures for treatment of a rat model of PD, resulting in an increased functional recovery. Related cell cultures and methods of treating brain diseases (e.g., PD) are also provided.

In some aspects, PD is treated in a subject by administering a cell replacement therapy of mDA cells that have been differentiated from induced pluripotent stem cells (iPSC). As shown in the below examples, in contrast to iPSC-derived post-mitotic mDA neurons, mDA progenitor cells were observed to yield superior results for the treatment of brain diseases involving cell transplantation therapy such as PD. The effects of cellular maturity on survival and efficacy of the transplants were examined by engrafting mDA progenitors (cryopreserved at 17 days of differentiation, D17), immature neurons (D24), and post-mitotic neurons (D37) into immunocompromised hemiparkinsonian rats. D17 progenitors were observed to be markedly superior to immature D24 or mature D37 neurons for cell survival, fiber outgrowth, and beneficial effects on motor deficits in vivo. Observed intranigral engraftment to the ventral midbrain demonstrated that D17 cells had a greater capacity than D24 cells to innervate over longer distances to forebrain structures, including the striatum. When D17 cells were tested across a wide dose range (7,500-450,000 injected cells per striatum), a clear dose response with regards to numbers of surviving neurons, innervation, and functional recovery was observed. Importantly, although these grafts were derived from iPSCs, no teratoma formation or significant outgrowth of other cells in any animal were observed. These data support the use of these iPSC-derived D17 mDA progenitor cells for clinical therapeutic treatment of PD.

### I. DEFINITIONS

“Pluripotency” or “pluripotent” refers to a stem cell or undifferentiated cell that has the potential to differentiate into all cells constituting one or more tissues or organs, for example, any of the three germ layers: endoderm (e.g., interior stomach lining, gastrointestinal tract, the lungs), mesoderm (e.g., muscle, bone, blood, urogenital), or ectoderm (e.g., epidermal tissues, nervous system).

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing or contacting the non-pluripotent cell with reprogramming factors.

“Embryonic stem (ES) cells” are pluripotent stem cells derived from early embryos.

“Adherent culture,” refers to a culture in which cells, or aggregates of cells, are attached to a surface.

“Suspension culture,” refers to a culture in which cells, or aggregates of cells, multiply while suspended in liquid medium.

“Essentially free” of an externally added component refers to a medium that does not have, or that have essentially none of, the specified component from a source other than the cells in the medium. “Essentially free” of externally added growth factors or signaling inhibitors, such as TGFβ, bFGF, TGFβ superfamily signaling inhibitors, etc., may mean a minimal amount or an undetectable amount of the externally added component. For example, a medium or environment essentially free of TGFβ or bFGF can contain less than 5, 4, 3, 2, 1, 0.9, 0.8, 0.7, 0.6, 0.5, 0.4, 0.3, 0.2, 0.1, 0.01, 0.001 ng/mL or any range derivable therein. For example, a medium or environment essentially free of signaling inhibitors can contain less than 0.2, 0.1, 0.09, 0.08, 0.07, 0.06, 0.05, 0.04, 0.03, 0.02, 0.01, 0.005, 0.001 μM, or any range derivable therein.

“Differentiation” is a process by which a less specialized cell forms progeny of at least a new cell type which is more specialized. For example, a stem cell may differentiate into a neuronal precursor cell, and the neuronal precursor cell may differentiate into a DA neuron.

The term “aggregate promoting medium” means any medium that enhances the aggregate formation of cells without any restriction as to the mode of action.

The term “aggregates,” i.e., embryoid bodies, refers to homogeneous or heterogeneous clusters of cells comprising differentiated cells, partly differentiated cells and/or pluripotent stem cells cultured in suspension.

“Neurons” or “neural cells” or “neural cell types” or “neural lineage” may include any neuron lineage cells, and can be taken to refer to cells at any stage of neuronal ontogeny without any restriction, unless otherwise specified. For example, neurons may include both neuron precursor cells and/or mature neurons. “Neural cells” or “neural cell types” and “neural lineage” cells can include any neuronal lineage and/or at any stage of neural ontogeny without restriction, unless otherwise specified. For example, neural cells can include neuron precursor cells, glial precursor cells, mature neurons, and/or glia.

A “gene,” “polynucleotide,” “coding region,” “sequence,” “segment,” or “fragment,” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded.

The term “transgene,” refers to a gene, nucleic acid, or polynucleotide which has been introduced into the cell or organism by artificial or natural means, such as an exogenous nucleic acid. An exogenous nucleic acid may be from a different organism or cell, or it may be one or more additional copies of a nucleic acid which occurs naturally within the organism or cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

As used herein “midbrain DA neuronal precursor cells,” “mDA neuronal precursor cells,” “mDA neuronal progenitor cells,” and “mDA precursor cells” are used interchangeably and refer to neuronal precursor cells that express FoxA2, Lmx1, and EN1 (a midbrain-specific marker); but the cells do not express Nurr1. Midbrain DA neuronal precursor cells may express one or more of: GBX2, OTX2, ETV5, DBX1TPH2, TH, BARHL1, SLC6A4, GATA2, NR4A2, GAD1, DCX, NXK6-1, RBFOX3, KCNJ6, CORIN, CD44, SPRY1, FABP7, SLC17A7, OTX1, and/or FGFR3. In some embodiments, the mDA precursor cells do express TH; for example, the mDA precursor cells may not yet express TH, but may retain the ability to express TH after additional differentiation. mDA precursor cells may express select genes at distinct stages of differentiation.

“Neural Stem Cell (NSCs)” are multipotent cells that can self-renew and proliferate potentially without limit, and may produce progeny cells that can terminally differentiate into neurons, astrocytes and/or oligodendrocytes. The non-stem cell progeny of NSCs are referred to as neural progenitor cells. “Neural Progenitor Cell” are progenitor cells that have the capacity to proliferate and differentiate into more than one cell type. Neural progenitor cells can be unipotent, bipotent or multipotent. A distinguishing feature of a neural progenitor cell is that, unlike a stem cell, it has a limited proliferative ability and does not exhibit self-renewal. “Neural Precursor Cells” (NPCs) refers to a mixed population of cells consisting of all undifferentiated progeny of neural stem cells, including both neural progenitor cells and neural stem cells. The term neural precursor cells can be used to describe the mixed population of NSCs and neural progenitor cells derived from embryonic stem cells or induced pluripotent stem cells.

### II. SMAD INHIBITORS FOR MONO-SMAD INHIBITION

In some aspects, pluripotent cells were differentiated using mono-SMAD methods for a period of about 360-456 hours, more preferably about 384-432 hours, to produce a culture of neural cells. In the mono-SMAD methods, a single SMAD inhibitor such as a single BMP signaling inhibitor or a single TGF-β signaling inhibitor is used to inhibit SMAD signaling in methods to convert pluripotent cells (e.g., iPS cells, ES cells) into neuronal cells such as midbrain dopaminergic cells. Generally, and in contrast to other dual-SMAD methods of differentiation, mono-SMAD differentiation methods utilize only a single SMAD inhibitor, and a second SMAD inhibitor is not included in the differentiation media. For example, in some aspects, pluripotent cells are converted into a population of neuronal precursor cells including midbrain DA neuronal precursor cells, wherein the differentiation occurs in a media comprising a single BMP signaling inhibitor. In some embodiments, the BMP inhibitor is LDN-193189, dorsomorphin, or DMH-1. Non-limiting examples of inhibitors of BMP signaling include dorsomorphin, dominant-negative BMP, truncated BMP receptor, soluble BMP receptors, BMP receptor-Fc chimeras, noggin, LDN-193189, follistatin, chordin, gremlin, cerberus/DAN family proteins, ventropin, high dose activin, and amnionless. In some embodiments, a nucleic acid, antisense, RNAi, siRNA, or other genetic method may be used to inhibit BMP signaling. As used herein, a BMP signaling inhibitor may be referred to simply as a “BMP inhibitor.” The BMP inhibitor may be included in the differentiation media on days 1, 2, 3, 4, 5, 6, 7, 8, 9, 10, 11, 12, 13, 14, 15, 16, and/or day 17 of differentiation, or any range derivable therein (e.g., days 1-17, 1-16, 1-15, 2-15, etc.). In some embodiments, the BMP inhibitor is included in the differentiation media on all of days 1-17 of differentiation. Nonetheless, it is anticipated that it may be possible to exclude the BMP inhibitor from the differentiation media at certain times, e.g., on 1, 2, or 3 of the above days. In some embodiments, the BMP inhibitor is optionally not included in the differentiation media on days 11-17, and in some preferred embodiments the BMP inhibitor is included in the differentiation media on days 1-10. Mono-SMAD methodologies are further discussed in WO2018/035214.

In some embodiments, the BMP inhibitor is LDN-193189, dorsomorphin, DMH-1, or noggin. For example, cells can be cultured in a media comprising about 1-2500, 1-2000, or 1-1,000 nM LDN-193189 (e.g., from about 10 to 500, 50 to 500, 50 to 300, 50, 100, 150, 200, 250, 300, 500, 750, 1000, 1250, 1500, 1750, 2000, 2250, or about 2500 nM LDN-193189, or any range derivable therein). In some embodiments, cells can be cultured in a media comprising about 0.1 to 10 μM dorsomorphin (e.g., from about 0.1 to 10, 0.5 to 7.5, 0.75 to 5, 0.5 to 3, 1 to 3, 0.25, 0.5, 0.75, 1, 1.25, 1.5, 1.55, 1.6, 1.65, 1.7, 1.75, 2, 2.25, 2.5, 2.75, 3, or about 2 μM dorsomorphin, or any range derivable therein). In some embodiments, cells can be cultured in a media comprising about 1 μM DMH-1 (e.g., about 0.2-8, 0.5-2, or about 1 μM DMH-1, or any range derivable therein). LDN-193189, dorsomorphin, and DMH-1 can be successfully used in mono-SMAD inhibition methods to produce midbrain dopaminergic neurons or mDA precursor cells from iPS cells. In some embodiments the BMP inhibitor is K 02288 or DMH2.

In some aspects, a TGFβ inhibitor may be used to inhibit SMAD in a mono-SMAD method to generate midbrain dopaminergic neurons or mDA precursor cells from pluripotent cells such as iPS cells. For example, in some embodiments, the differentiation media comprises a TGFβ signaling inhibitor. Non-limiting examples of inhibitors of TGFβ signaling include LDN-193189, SB-525334, GW788388, A-83-01, GW6604, IN-1130, Ki26894, LY2157299, LY364947 (HTS-466284), A-83-01, LY550410, LY573636, LY580276, NPC-30345, SB-431542, SB-505124, SD-093, Sml6, SM305, SX-007, Antp-Sm2A, and LY2109761. For instance, the TGFβ inhibitor in a differentiation media may be SB431542. In some aspects, cells are cultured in a media comprising about 0.1 to 100 μM SB431542 (e.g., between about 1 to 100, 10 to 80, 15 to 60, 20-50, or about 40 μM SB431542). As used herein, a TGFβ signaling inhibitor, including a TGFβ receptor inhibitor, may be referred to simply as a “TGFβ inhibitor.” In some embodiments, a TGFβ inhibitor is not included in the differentiation media. In some embodiments, a TGFβ inhibitor (e.g., SB431542) be included in a differentiation media on days 1-3, or 1, 2, 3, and/or day 4 as the mono-SMAD inhibitor. As shown in the below examples, in some embodiments, a BMP inhibitor is used as the mono-SMAD inhibitor since these compounds were observed to produce superior differentiation of pluripotent cells into midbrain DA neurons or mDA precursor cells, as compared to use of a TGFβ inhibitor.

### III. INCLUSION OF MEK INHIBITOR

In some aspects, a MEK inhibitor is included in a differentiation media, e.g., in combination with the BMP inhibitor or mono-SMAD inhibitor to produce midbrain dopaminergic neurons or mDA precursor cells from pluripotent cells such as iPS cells. In some embodiments, the MEK inhibitor is PD0325901. Non-limiting examples of MEK inhibitors that could be used include PD0325901, trametinib (GSK1120212), selumetinib (AZD6244), pimasertib (AS-703026), MEK162, cobimetinib, PD184352, PD173074, BIX 02189, AZD8330 and PD98059. For example, in some embodiments, the method comprises culturing the cells in the presence of between about 0.1 and 10 μM (e.g., between about 0.1 and 5; 0.5 and 3 or 0.5 and 1.5 μM) of the MEK inhibitor, such as PD0325901. In some embodiments, cells are contacted with the MEK inhibitor (e.g., PD0325901) on day 3, 4, 5, or days 3-5 of the differentiation.

Thus, in certain aspects, differentiating the cells comprises culturing a population of pluripotent cells in a media comprising a BMP inhibitor, an activator of Sonic hedgehog (SHH) signaling, an activator of Wnt signaling, a MEK inhibitor or a combination of the foregoing, wherein the media does not contain exogenously added FGF8b. In some instances, a TGFβ inhibitor may be used instead of a BMP inhibitor. In some embodiments, the method does not comprise purification of cells using a DA-specific marker. In some aspects, the pluripotent cells comprise a resistance gene under the control of a neuronal promoter that may be used for the purification of neuronal cells (e.g., neuronal cells expressing an antibiotic resistance gene will survive exposure to the antibiotic, whereas non-neuronal cells will die).

In some embodiments, midbrain DA neuronal precursor cells may be produced by a method comprising: obtaining a population of pluripotent cells; differentiating the cells into a neural lineage cell population in a medium comprising a MEK inhibitor (e.g., PD0325901), wherein the medium does not contain exogenously added FGF8b on day 1 of the differentiation; and further differentiating cells of the neural lineage cell population to provide an enriched population of midbrain DA neurons or mDA precursor cells. In some embodiments, it has been observed that inclusion of FGF8 (e.g., FGF8b) in the differentiation media on day 1 can, in some instances, impede or prevent differentiation of the cells into midbrain DA neuronal precursor cells. In some embodiments, FGF8 may optionally be included in a differentiation media on later days of differentiation such as, e.g., days 9, 10, 11, 12, 13, 14, 15, 16, 17, or any range derivable therein, e.g., preferably wherein contact of pluripotent cells is initiated with the single SMAD inhibitor in a differentiation media on day 1.

### IV. INCLUSION OF WNT ACTIVATOR OR GSK INHIBITOR

In some aspects, a Wnt activator (e.g., a GSK3 inhibitor) is included in a differentiation media, e.g., in combination with the BMP inhibitor or mono-SMAD inhibitor to generate midbrain dopaminergic neuronal precursor cells from pluripotent cells such as iPS cells. In some embodiments, pluripotent cells into a population of neuronal cells comprising midbrain DA neurons or mDA precursor cells, wherein the differentiation is in a media comprising at least a first activator of Wnt signaling.

A variety of Wnt activators or GSK3 inhibitors may be used in various aspects of the present disclosure. For example, the activator of WNT signaling can be a glycogen synthase kinase 3 (GSK3) inhibitor. Non-limiting examples of GSK3 inhibitors include NP031112, TWS119, SB216763, CHIR-98014, AZD2858, AZD1080, SB415286, LY2090314 and CHIR99021. In some embodiments, pluripotent cells are contacted with a single SMAD inhibitor that is not SB415286. In some embodiments, the activator of Wnt signaling is CHIR99021. Thus, in some aspects, a culture media for use according to the embodiments comprises from about 0.1 to about 10 μM CHIR99021 (e.g., between about 0.1 to 5, 0.5 to 5, 0.5 to 3, from greater than about 1.25 to 2.25, about 1.25, 1.5, 1.55, 1.65, 1.7, 1.75, 1.8, 1.9, 2.0, or about 1.75 μM CHIR99021, or any range derivable therein). In some preferred embodiments, about 1.6-1.7 μM, or about 1.65 μM of CHIR99021 is used.

In some preferred embodiments, the Wnt activator (e.g., GSK3 inhibitor) is optionally not included in the differentiation media on day 1 of differentiation. In some embodiments, the Wnt activator or GSK inhibitor (e.g., CHR99021) is included in the differentiation media on days 2, 3, 4, 5, 6, 7, 8, 9, 10, 11, 12, 13, 14, 15, 16, and/or day 17, or any combination or all of these days. For example, in some embodiments, the Wnt activator or GSK inhibitor is included in the differentiation media on days 2-17 or days 3-17.

### V. SONIC HEDGEHOG ACTIVATOR

In some aspects, an activator of Sonic hedgehog (SHH) signaling is included in a differentiation media, e.g., in combination with the BMP inhibitor or mono-SMAD inhibitor to generate midbrain dopaminergic neurons or mDA precursor cells from pluripotent cells such as iPS cells. In some embodiments, the Sonic Hedgehog activator is Sonic Hedgehog (Shh) or a mutant Shh. The Shh can be, e.g., a human or mouse protein or it may be derived from a human or mouse Shh. For example, in some embodiments, the Shh is a mutant mouse Shh protein such as mouse C25II Shh or human C24II Shh. In some embodiments, the differentiation media comprises both Shh (e.g., C25II Shh) and a small molecule activator of SHH such as, e.g., purmorphamine. Without wishing to be bound by any theory, the Shh and/or activator of Sonic Hedgehog may promote neural floor plate differentiation.

In some embodiments, mDA precursor cells are generated from pluripotent cells by a method comprising culturing the pluripotent cells in a media comprising at least a first activator of SHH signaling. For example, the activator of SHH signaling can be a recombinant SHH polypeptide (or a portion thereof) or a small molecule activator. In certain aspects, the activator of SHH may be Shh C25II, purmorphamine, or a purmorphamine analogue (e.g., a Smoothened agonist, such as SAG-1 or 3-chloro-N-[(1r,4r)-4-(methylamino)cyclohexyl]-N-[3-(pyridin-4-yl)benzyl]benzo[b]thiophene-2-carboxamide). Thus, in certain aspects, a culture media for use according to the embodiments comprises about 0.1 to 10 UM purmorphamine (e.g., between about 0.1 to 20, 0.5 to 10, 0.5 to 5 or about 2 μM purmorphamine). In further aspects, a culture media comprises about 1 to 1,000 ng/ml Shh C25II (e.g., about 10 to 1,000, 10 to 500, 50 to 500 or about 100 ng/ml Shh C25II). In some embodiments, the activator of SHH signaling includes both Shh C25II and purmorphamine. For example, cells may be cultured in a media comprising about 0.1 to 10 UM purmorphamine and about 1 to 1,000 ng/ml Shh C25II. The SHH activator(s) (e.g., Shh C25II and purmorphamine) may be included in a differentiation media on days 1, 2, 3, 4, 5, 6, and/or 7. In some embodiments, the SHH activators are excluded from the differentiation media on day 1. For example, in various embodiments, the SHH activator(s) are included in the differentiation media on days 1-6 or 2-7.

Thus, in certain aspects, pluripotent cells may be cultured in a differentiation for 1-6 days in an adherent culture system with a DMEM/F12 media comprising B27 supplement, 1-3000 or 1-1000 nM LDN-193189 (or 0.1 to 100 μM SB431542), 0.1 to 50 μM purmorphamine, 1 to 1,000 ng/ml Shh C25II, and 0.1 to 10 μM CHIR99021. In one aspect, the media may comprise B27 supplement, 200 nM LDN-193189 (or 10 μM SB431542), 2 μM purmorphamine, 100 ng/ml Shh C25II, and 1.25 μM CHIR99021. In some embodiments, the MEK inhibitor is included in the media after 1-2 days (e.g., the MEK inhibitor is included on days 2-4, or days 2, 3, and/or 4 of differentiation).

### VI. SOURCES OF PLURIPOTENT STEM CELLS

Pluripotent stem cells may be used in the methods disclosed herein for neural induction. Methods and compositions are disclosed herein that may be used, e.g., to produce midbrain DA neuronal precursor cells with improved therapeutic properties (e.g., for the treatment of a neurodegenerative disease such as PD).

The term “pluripotent stem cell” or “pluripotent cell” refers to a cell capable of giving rise to cells of all three germinal layers, that is, endoderm, mesoderm and ectoderm. Although in theory a pluripotent stem cell can differentiate into any cell of the body, the experimental determination of pluripotency is typically based on differentiation of a pluripotent cell into several cell types of each germinal layer. In some embodiments, the pluripotent stem cell is an embryonic stem (ES) cell derived from the inner cell mass of a blastocyst. In other embodiments, the pluripotent stem cell is an induced pluripotent stem cell derived by reprogramming somatic cells. In some embodiments, the pluripotent stem cell is an embryonic stem cell derived by somatic cell nuclear transfer. The pluripotent stem cell may be obtained or derived from a healthy subject (e.g., a healthy human) or a subject with a disease (e.g., a neurodegenerative disease, Parkinson's disease, etc.).

### A. Embryonic Stem Cells

Embryonic stem (ES) cells are pluripotent cells derived from the inner cell mass of a blastocyst. ES cells can be isolated by removing the outer trophectoderm layer of a developing embryo, then culturing the inner mass cells on a feeder layer of non-growing cells. Under appropriate conditions, colonies of proliferating, undifferentiated ES cells are produced. The colonies can be removed, dissociated into individual cells, and then replated on a fresh feeder layer. The replated cells can continue to proliferate, producing new colonies of undifferentiated ES cells. The new colonies can then be removed, dissociated, replated again and allowed to grow. This process of “subculturing” or “passaging” undifferentiated ES cells can be repeated to produce cell lines containing undifferentiated ES cells (e.g., as described in U.S. Pat. Nos. 5,843,780; 6,200,806; 7,029,913). A “primary cell culture” is a culture of cells directly obtained from a tissue such as, e.g., the inner cell mass of a blastocyst. A “subculture” is any culture derived from the primary cell culture.

Methods for obtaining mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be obtained from blastocysts using previously described methods (Thomson et al., 1995; Thomson et al., 1998; Thomson and Marshall, 1998; Reubinoff et al, 2000.) In one method, day-5 human blastocysts are exposed to rabbit anti-human spleen cell antiserum, and then exposed to a 1:5 dilution of Guinea pig complement to lyse trophectoderm cells. After removing the lysed trophectoderm cells from the intact inner cell mass, the inner cell mass is cultured on a feeder layer of gamma-inactivated mouse embryonic fibroblasts and in the presence of fetal bovine serum. After 9 to 15 days, clumps of cells derived from the inner cell mass can be chemically (e.g., exposed to trypsin) or mechanically dissociated and replated in fresh medium containing fetal bovine serum and a feeder layer of mouse embryonic fibroblasts. Upon further proliferation, colonies having undifferentiated morphology are selected by micropipette, mechanically dissociated into clumps, and replated (see U.S. Pat. No. 6,833,269). ES-like morphology is characterized as compact colonies with apparently high nucleus to cytoplasm ratio and prominent nucleoli. Resulting ES cells can be routinely passaged by brief trypsinization or by selection of individual colonies by micropipette. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as Matrigel™ or laminin in the presence of “conditioned” medium containing basic fibroblast growth factor (Xu et al., 2001). The medium can be previously conditioned by coculturing with fibroblasts.

Methods for the isolation of rhesus monkey and common marmoset ES cells are also known (Thomson, and Marshall, 1998; Thomson et al., 1995; Thomson and Odorico, 2000).

Another source of ES cells are established ES cell lines. Various mouse cell lines and human ES cell lines are known and conditions for their growth and propagation have been defined. For example, the mouse CGR8 cell line was established from the inner cell mass of mouse strain 129 embryos, and cultures of CGR8 cells can be grown in the presence of LIF without feeder layers. As a further example, human ES cell lines H1, H7, H9, H13 and H14 were established by Thomson et al. (2000). In addition, subclones H9.1 and H9.2 of the H9 line have been developed. It is anticipated that virtually any ES or stem cell line known in the art may be used with the present disclosure, such as, e.g., those described in Yu and Thomson, 2008, which is incorporated herein by reference.

The source of ES cells may include a blastocyst, cells derived from culturing the inner cell mass of a blastocyst, and cells obtained from cultures of established cell lines. Thus, as used herein, the term “ES cells” can refer to inner cell mass cells of a blastocyst, ES cells obtained from cultures of inner mass cells, and ES cells obtained from cultures of ES cell lines.

### B. Induced Pluripotent Stem Cells

Induced pluripotent stem (iPS) cells have characteristics of ES cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi et al., 2006, 2007). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF. In some preferred embodiments, the iPS cells are human iPS cells.

The induced pluripotent stem cells are morphologically similar to human ES cells and express various human ES cell markers. When grown under conditions that are known to result in differentiation of human ES cells, the induced pluripotent stem cells differentiate accordingly. For example, the induced pluripotent stem cells can differentiate into cells having neuronal structures and neuronal markers. It is anticipated that virtually any iPS cell or cell lines may be used with the present disclosure, including, e.g., those described in Yu and Thomson, 2008. As would be appreciated by one of skill, a variety of iPS cell lines have been generated, and iPS cells from these established cell lines can be used in various embodiments of the present disclosure.

In another method, human fetal or newborn fibroblasts are transfected with four genes, Oct4, Sox2, Nanog and Lin28 using lentivirus transduction (Yu et al., 2007). At 12-20 days post infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage and gut epithelium after injection into mice.

Methods of preparing induced pluripotent stem cells from mouse cells are also known (Takahashi and Yamanaka, 2006). Induction of iPS cells typically requires the expression of or exposure to at least one member from the Sox family and at least one member from the Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc.

iPS cells, like ES cells, have characteristic antigens that can be identified or confirmed by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by, e.g., by injecting approximately 0.5-10×10⁶ cells into the rear leg muscles of 8-12 week old male SCID mice. Teratomas develop that demonstrate at least one cell type of each of the three germ layers. iPS cells can be generated using somatic cells that have been modified to express reprogramming factors comprising an Oct family member and a Sox family member, such as Oct4 and Sox2 in combination with Klf or Nanog, e.g., as described above. The somatic cell may be any somatic cell that can be induced to pluripotency such as, e.g., a fibroblast, a keratinocyte, a hematopoietic cell, a mesenchymal cell, a liver cell, a stomach cell, or a β cell. In some embodiments, T cells may also be used as source of somatic cells for reprogramming (e.g., see WO 2010/141801, incorporated herein by reference).

Reprogramming factors may be expressed from expression cassettes comprised in one or more vectors, such as an integrating vector, a chromosomally non-integrating RNA viral vector (see U.S. application Ser. No. 13/054,022, incorporated herein by reference) or an episomal vector, such as an EBV element-based system (e.g., see WO 2009/149233, incorporated herein by reference; Yu et al., 2009). In a further aspect, reprogramming proteins or RNA (such as mRNA or miRNA) could be introduced directly into somatic cells by protein or RNA transfection (Yakubov et al., 2010).

### C. Embryonic Stem Cells Derived by Somatic Cell Nuclear Transfer

Pluripotent stem cells can be prepared by means of somatic cell nuclear transfer, in which a donor nucleus is transferred into a spindle-free oocyte. Stem cells produced by nuclear transfer are genetically identical to the donor nuclei. Methods for generating embryonic stem cells derived by somatic cell nuclear transfer are provided in Tachibana et al., 2013. As used herein, the term “ES cells” refers to embryonic stem cells derived from embryos containing fertilized nuclei, and embryonic stem cells produced by nuclear transfer are referred to as “NT-ESCs.”

### VII. MEDIUM FOR DIFFERENTIATION

A differentiation medium according to certain aspects of the present disclosure can be prepared using a medium to be used for culturing animal cells as its basal medium. In some embodiments, a differentiation medium is used to differentiate pluripotent cells into midbrain dopaminergic neuronal precursor cells (e.g., D17 cells) using only a single BMP inhibitor or a single TGF-beta inhibitor. For example, a differentiation medium used to promote differentiation of pluripotent cells (e.g., into midbrain dopaminergic precursor cells) may comprise a single BMP inhibitor (such as LDN-193189 or dorsomorphin; e.g., on days 1-17 of differentiation; an activator of Sonic hedgehog (SHH) signaling (such as purmorphamine, human C25II SHH, or mouse C24II SHH; e.g., on days 1-6, 2-7, or 1-7); an activator of Wnt signaling (such as a GSK inhibitor, e.g., CHIR99021; e.g., on days 2-17 or 3-17) and/or a MEK inhibitor (such as PD0325901; e.g., on days 2-4 or 3-5). In some embodiments, a single TGFβ inhibitor (such as SB-431542; e.g., on days 1-4) may be used instead of the single BMP inhibitor; however, in some embodiments a single BMP inhibitor may result in superior differentiation of cells into FOXA2⁺/LMX1A⁺, cells as compared to use of a single TGF-β inhibitor. In some embodiments, FGF-8 (e.g., FGF-8b) is not included in differentiation media on the first day or days 1, 2, 3, 4, 5, 6, 7, 8, 9, or 10, or any combination thereof (e.g., days 1-8); for example, in some embodiments, FGF-8 is included in the differentiation media on days 9, 10, 11, 12, 13, 14, 15, 16, and 17, or any combination thereof. In various embodiments, the differentiation media may contain TGFβ and bFGF, or, alternately, the differentiation media may be essentially free of TGFβ and bFGF.

In certain aspects, a method of differentiation according to the embodiments involves passage of cell through a range of media conditions for example cells are cultured
in adherent culture in a medium comprising: a single BMP inhibitor (or a TGFβ inhibitor); an activator of Sonic hedgehog (SHH) signaling; and an activator of Wnt signaling;
in suspension in a medium comprising a single BMP inhibitor (or a TGFβ inhibitor); an activator of SHH signaling; and an activator of Wnt signaling, wherein cell aggregates are formed;
in adherent culture in a Neurobasal medium comprising B27 supplement, L-glutamine, BDNF, GDNF, TGFβ, ascorbic acid, dibutyryl cAMP, and DAPT, (and, optionally, lacking exogenously added retinol or retinoic acid) for maturation.

As the basal medium, any chemically defined medium, such as Eagle's Basal Medium (BME), BGJb, CMRL 1066, Glasgow MEM, Improved MEM Zinc Option, Iscove's modified Dulbecco's medium (IMDM), Medium 199, Eagle MEM, αMEM, DMEM, Ham, RPMI 1640, and Fischer's media, variations or combinations thereof can be used, wherein TGFβ and bFGF may or may not be included.

In further embodiments, the cell differentiation environment can also contain supplements such as B-27 supplement, an insulin, transferrin, and selenium (ITS) supplement, L-Glutamine, NEAA (non-essential amino acids), P/S (penicillin/streptomycin), N2 supplement (5 μg/mL insulin, 100 μg/mL transferrin, 20 nM progesterone, 30 nM selenium, 100 μM putrescine (Bottenstein, and Sato, 1979 PNAS USA 76, 514-517) and/or β-mercaptoethanol (β-ME). It is contemplated that additional factors may or may not be added, including, but not limited to fibronectin, laminin, heparin, heparin sulfate, retinoic acid.

Growth factors may or may not be added to a differentiation medium. In addition or in place of the factors outlined above, growth factors such as members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) family antagonists may be employed at various steps in the process. In some embodiments, FGF-8 is included in a differentiation media as described herein. Other factors that may or may not be added to the differentiation media include molecules that can activate or inactivate signaling through Notch receptor family, including but not limited to proteins of the Delta-like and Jagged families as well as gamma secretase inhibitors and other inhibitors of Notch processing or cleavage such as DAPT. Other growth factors may include members of the insulin like growth factor family (IGF), the wingless related (WNT) factor family, and the hedgehog factor family.

Additional factors may be added in an aggregate formation and/or differentiation medium to promote neural stem/progenitor proliferation and survival as well as neuron survival and differentiation. These neurotrophic factors include but are not limited to nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the glial derived neurotrophic factor (GDNF) family including but not limited to neurturin, neublastin/artemin, and persephin and factors related to and including hepatocyte growth factor. Neural cultures that are terminally differentiated to form post-mitotic neurons may also contain a mitotic inhibitor or mixture of mitotic inhibitors including but not limited to 5-fluoro 2′-deoxyuridine, Mitomycin C and/or cytosine β-D-arabino-furanoside (Ara-C).

The medium can be a serum-containing or serum-free medium. The serum-free medium may refer to a medium with no unprocessed or unpurified serum and accordingly, can include media with purified blood-derived components or animal tissue-derived components (such as growth factors). From the aspect of preventing contamination with heterogeneous animal-derived components, serum can be derived from the same animal as that of the stem cell(s). In some embodiments, the medium is a defined medium, and the medium does not contain serum or other animal tissue-derived components (such as irradiated mouse fibroblasts or a media that has been conditioned with irradiated fibroblast feeder cells).

The medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolglycerol, or equivalents thereto. For example, an alternative to serum may be prepared by the method disclosed in International Publication No. 98/30679. Alternatively, commercially available materials can be used for more convenience. The commercially available materials include knockout Serum Replacement (KSR) and Chemically-defined Lipid concentrate (Gibco).

The medium can also contain fatty acids or lipids, amino acids (such as non-essential amino acids), vitamin(s), growth factors, cytokines, antioxidant substances, 2-mercaptoethanol, pyruvic acid, buffering agents, and inorganic salts. The concentration of 2-mercaptoethanol can be, for example, about 0.05 to 1.0 mM, and particularly about 0.1 to 0.5, or 0.01, 0.02, 0.03, 0.04, 0.05, 0.1, 0.2, 0.5, 0.8, 1, 1.5, 2, 2.5, 5, 7.5, 10 mM or any intermediate values, but the concentration is particularly not limited thereto as long as it is appropriate for culturing the stem cell(s).

In some embodiments, pluripotent stem cells are cultured in a medium prior to aggregate formation to improve neural induction and floor plate patterning (e.g., prior to being dissociated into single cells or small aggregates to induce aggregate formation). In certain embodiments of the invention, the stem cells may be cultured in the absence of feeder cells, feeder cell extracts and/or serum.

### B. Culture Conditions

A culture vessel used for culturing the cell(s) can include, but is particularly not limited to: flask, flask for tissue culture, spinner flask, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the cells therein. The cells may be cultured in a volume of at least or about 0.2, 0.5, 1, 2, 5, 10, 20, 30, 40, 50, 100, 150, 200, 250, 300, 350, 400, 450, 500, 550, 600, 800, 1000, 1500 mL, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel surface can be prepared with cellular adhesive or not depending upon the purpose. The cellular adhesive culture vessel can be coated with any substrate for cell adhesion such as extracellular matrix (ECM) to improve the adhesiveness of the vessel surface to the cells. The substrate used for cell adhesion can be any material intended to attach stem cells or feeder cells (if used). Non-limiting substrates for cell adhesion include collagen, gelatin, poly-L-lysine, poly-D-lysine, poly-L-ornithine, laminin, vitronectin, and fibronectin and mixtures thereof, for example, protein mixtures from Engelbreth-Holm-Swarm mouse sarcoma cells (such as Matrigel™ or Geltrex) and lysed cell membrane preparations (Klimanskaya et al., 2005). In some embodiments, the cellular adhesive culture vessel is coated with a cadherin protein, e.g., epithelial cadherin (E-cadherin).

Other culturing conditions can be appropriately defined. For example, the culturing temperature can be about 30 to 40° C., for example, at least or about 31, 32, 33, 34, 35, 36, 37, 38, 39ºC but particularly not limited to them. The CO₂ concentration can be about 1 to 10%, for example, about 2 to 7%, or any range derivable therein. The oxygen tension can be at least or about 1, 5, 8, 10, 20%, or any range derivable therein.

An adhesion culture may be used in certain aspects. If desired, the cells can be cultured in the presence of feeder cells. In the case where the feeder cells are used, stromal cells such as fetal fibroblasts can be used as feeder cells (for example, refer to; Manipulating the Mouse Embryo A Laboratory Manual (1994); Gene Targeting, A Practical Approach (1993); Martin (1981); Evans et al. (1981); Jainchill et al., (1969); Nakano et al., (1996); Kodama et al. (1982); and International Publication Nos. 01/088100 and 2005/080554). In some embodiments, feeder cells are not included in the cell culture media, and cells may be cultured using defined conditions.

In other aspects, a suspension culture may be used. Suspension cultures that may be used include a suspension culture on carriers (Fernandes et al., 2007) or gel/biopolymer encapsulation (U.S. Patent Publication No. 2007/0116680). Suspension culture of stem cells generally involves culture of cells (e.g., stem cells) under non-adherent conditions with respect to the culture vessel or feeder cells (if used) in a medium. Suspension cultures of stem cells generally include dissociation cultures of stem cells and aggregate suspension cultures of stem cells. Dissociation cultures of stem cells involve culture of suspended stem cells, such as single stem cells or those of small cell aggregates composed of a plurality of stem cells (for example, about 2 to 400 cells). When the dissociation culture is continued, the cultured, dissociated cells normally form a larger aggregate of stem cells, and thereafter an aggregate suspension culture can be produced or utilized. Aggregate suspension culture methods include embryoid culture methods (see Keller et al., 1995), and a SFEB (serum-free embryoid body) methods (Watanabe et al., 2005); International Publication No. 2005/123902).

### C. Culturing of Pluripotent Stem Cells

Methods for preparing and culturing pluripotent stem cells such as ES cells can be found in standard textbooks and reviews in cell biology, tissue culture, and embryology, including teratocarcinomas and embryonic stem cells: Guide to Techniques in Mouse Development (1993); Embryonic Stem Cell Differentiation in vitro (1993); Properties and uses of Embryonic Stem Cells: Prospects for Application to Human Biology and Gene Therapy (1998), all incorporated herein by reference. Standard methods used in tissue culture generally are described in Animal Cell Culture (1987); Gene Transfer Vectors for Mammalian Cells (1987); and Current Protocols in Molecular Biology and Short Protocols in Molecular Biology (1987 & 1995).

After somatic cells are introduced into or contacted with reprogramming factors, these cells may be cultured in a medium sufficient to maintain the pluripotency and the undifferentiated state. Culturing of induced pluripotent stem (iPS) cells can use various medium and techniques developed to culture primate pluripotent stem cells, embryonic stem cells, or iPS cells, for example as described in U.S. Pat. Publication 2007/0238170 and U.S. Pat. Publication 2003/0211603, and U.S. Pat. Publication 2008/0171385, which are hereby incorporated by reference. It is appreciated that additional methods for the culture and maintenance of pluripotent stem cells, as would be known to one of skill, can be used.

In certain embodiments, undefined conditions may be used; for example, pluripotent cells may be cultured on fibroblast feeder cells or a medium that has been exposed to fibroblast feeder cells in order to maintain the stem cells in an undifferentiated state. Alternately, pluripotent cells may be cultured and maintained in an essentially undifferentiated state using defined, feeder-independent culture system, such as a TeSR medium (Ludwig et al., 2006a; Ludwig et al., 2006b) or E8 medium (Chen et al., 2011; PCT/US2011/046796). Feeder-independent culture systems and media may be used to culture and maintain pluripotent cells. These approaches allow human pluripotent stem cells to remain in an essentially undifferentiated state without the need for mouse fibroblast “feeder layers.”

Various matrix components may be used in culturing, maintaining, or differentiating human pluripotent stem cells. For example, collagen IV, fibronectin, laminin, and vitronectin in combination may be used to coat a culturing surface as a means of providing a solid support for pluripotent cell growth, as described in Ludwig et al. (2006a; 2006b), which are incorporated by reference in their entirety.

Matrigel™ may also be used to provide a substrate for cell culture and maintenance of human pluripotent stem cells. Matrigel™ is a gelatinous protein mixture secreted by mouse tumor cells and is commercially available from BD Biosciences (New Jersey, USA). This mixture resembles the complex extracellular environment found in many tissues and is used by cell biologists as a substrate for cell culture. In some embodiments, E-cadherin (e.g., recombinant E-cadherin substratum) is provided as a substrate for the culture and maintenance of the pluriporent cells, such as human pluripotent cells or human iPS cells. Related methods are provided, e.g., in Nagaoka et al. (2010).

### D. Single Cell Passaging

In some embodiments of pluripotent stem cell culturing, once a culture container is full, the colony is split into aggregated cells or even single cells by any method suitable for dissociation, which cells are then placed into new culture containers for passaging. Cell passaging or splitting is a technique that enables cells to survive and grow under cultured conditions for extended periods of time. Cells typically would be passaged when they are about 70%-100% confluent.

Single-cell dissociation of pluripotent stem cells followed by single cell passaging may be used in the present methods with several advantages, like facilitating cell expansion, cell sorting, and defined seeding for differentiation and enabling automatization of culture procedures and clonal expansion. For example, progeny cells clonally derived from a single cell may be homogenous in genetic structure and/or synchronized in cell cycle, which may increase targeted differentiation. Exemplary methods for single cell passaging may be as described in US 2008/0171385, which is incorporated herein by reference.

In certain embodiments, pluripotent stem cells may be dissociated into single individual cells, or a combination of single individual cells and small cell clusters comprising 2, 3, 4, 5, 6, 7, 8, 9, 10 cells or more. The dissociation may be achieved by mechanical force, or by a cell dissociation agent, such as a chelating agent, sodium citrate (Na Citrate), or an enzyme, e.g., trypsin, trypsin-EDTA, Accutase, TrypLE Select, or the like. Dissociation of cells may be achieved using chemical separation (e.g., using a chelator or enzyme) and/or mechanical agitation to dissociate cells.

Based on the source of pluripotent stem cells and the need for expansion, the dissociated cells may be transferred individually or in small clusters to new culture containers in a splitting ratio such as at least or about 1:2, 1:4, 1:5, 1:6, 1:8, 1:10, 1:20, 1:40, 1:50, 1:100, 1:150, 1:200, or any range derivable therein. Suspension cell line split ratios may be done on volume of culture cell suspension. The passage interval may be at least or about every 1, 2, 3, 4, 5, 6, 7, 8, 9, 10, 11, 12, 13, 14, 15, 16, 17, 18, 19, 20 days or any range derivable therein. For example, the achievable split ratios for the different enzymatic passaging protocols may be 1:2 every 3-7 days, 1:3 every 4-7 days, and 1:5 to 1:10 approximately every 7 days, 1:50 to 1:100 every 7 days. When high split ratios are used, the passage interval may be extended to at least 12-14 days or any time period without cell loss due to excessive spontaneous differentiation or cell death.

In certain aspects, single cell passaging may be in the presence of a small molecule effective for increasing cloning efficiency and cell survival, such as a ROCK inhibitor or myosin II inhibitor. The ROCK inhibitor or myosin II inhibitor, e.g., Y-27632, HA-1077, H-1152, or blebbistatin, may be used at an effective concentration, for example, at least or about 0.02, 0.05, 0.1, 0.2, 0.5, 1, 2, 3, 4, 5, 10, 15, 20, 25, 30, 35, 40, 45, 50 to about 100 μM, or any range derivable therein.

### E. Differentiation of Stem Cells

Methods are provided herein for generating mDA precursor cells with improved therapeutic properties (e.g., for treating Parkinson's disease, etc.). Differentiation of pluripotent stem cells can be induced in a variety of manners, such as in attached colonies or by formation of cell aggregates, e.g., in low-attachment environment, wherein those aggregates are referred to as embryoid bodies (EBs). The molecular and cellular morphogenic signals and events within EBs mimic many aspects of the natural ontogeny of such cells in a developing embryo. Methods for directing cells into neuronal differentiation are provided for example in U.S. Publn. No. 2012/0276063, incorporated herein by reference. More detailed and specific protocols for DA neuron differentiation are provided in PCT Publication No. WO2013/067362, incorporated herein by reference.

Embryoid bodies (EBs) are aggregates of cells that can be derived from pluripotent stem cells, such as ES cells or iPS cells, and have been studied with mouse embryonic stem cells. In order to recapitulate some of the cues inherent to in vivo differentiation, three-dimensional aggregates (i.e., embryoid bodies) may be generated as an intermediate step. Upon the start of cell aggregation, differentiation may be initiated, and the cells may begin to a limited extent to recapitulate embryonic development. Though they cannot form trophectodermal tissue (which includes the placenta), cells of virtually every other type present in the organism can develop. Neural differentiation can be promoted following aggregate formation.

Cell aggregation may be imposed by hanging drop, plating upon non-tissue culture treated plates or spinner flasks; either method prevents cells from adhering to a surface to form the typical colony growth. ROCK inhibitors or myosin II inhibitors may be used before, during or after aggregate formation to culture pluripotent stem cells.

Pluripotent stem cells may be seeded into aggregate promotion medium using any method known in the art of cell culture. For example, pluripotent stem cells may be seeded as a single colony or clonal group into aggregate promotion medium, and pluripotent stem cells may also be seeded as essentially individual cells. In some embodiments, pluripotent stem cells are dissociated into essentially individual cells using mechanical or enzymatic methods known in the art. By way of non-limiting example, pluripotent stem cells may be exposed to a proteolytic enzyme which disrupts the connections between cells and the culturing surface and between the cells themselves. Enzymes which may be used to individualize pluripotent stem cells for aggregate formation and differentiation may include, but are not limited to, trypsin, in its various commercial formulations, such as TrypLE, or a mixture of enzymes such as Accutase®. In certain embodiments, pluripotent cells may be added or seeded as essentially individual (or dispersed) cells to a culturing medium for culture formation on a culture surface.

For example, dispersed pluripotent cells may be seeded into a culturing medium. In these embodiments, a culturing surface may be comprised of essentially any material which is compatible with standard aseptic cell culture methods in the art, for example, a non-adherent surface. A culturing surface may additionally comprise a matrix component as described herein. In some embodiments, a matrix component may be applied to a culturing surface before contacting the surface with cells and medium.

Substrates that may be used to induce differentiation such as collagen, fibronectin, vitronectin, laminin, matrigel, and the like. Differentiation can also be induced by leaving the cells in suspension in the presence of a proliferation-inducing growth factor, without reinitiating proliferation (i.e., without dissociating the neurospheres).

In some embodiments, cells are cultured on a fixed substrate in a culture medium. A proliferation-inducing growth factor can then be administered to the cells. The proliferation inducing growth factor can cause the cells to adhere to the substrate (e.g., polyornithine-treated plastic or glass), flatten, and begin to differentiate into different cell types.

### V. NON-STATIC CULTURE

In certain aspects, non-static culture could be used for culturing and differentiation of pluripotent stem cells. The non-static culture can be any culture with cells kept at a controlled moving speed, by using, for example, shaking, rotating, or stirring platforms or culture vessels, particularly large-volume rotating bioreactors. In some embodiments, a rocker table may be used. The agitation may improve circulation of nutrients and cell waste products, and may also control cell aggregation by providing a more uniform environment. For example, rotary speed may be set to at least or at most about 10, 15, 20, 25, 30, 35, 40, 45, 50, 75, 100 rpm, or any range derivable therein. The incubation period in the non-static culture for pluripotent stem cells, cell aggregates, differentiated stem cells, or progeny cells derived therefrom, may be at least or about 4 hours, 8 hours, 16 hours, or 1, 2, 3, 4, 5, 6 days, or 1, 2, 3, 4, 5, 6, 7 weeks, or any range derivable therein.

### VI. GENETIC ALTERATION AND PURIFICATION OF CELLS

In some embodiments, cell provided herein such as mDA precursor cells can be genetically altered. A cell is said to be “genetically altered” or “transgenic” when a polynucleotide has been transferred into the cell by any suitable means of artificial manipulation, or where the cell is a progeny of the originally altered cell that has inherited the polynucleotide. In some embodiments, cells may comprise an antibiotic resistance gene, e.g., under the control of a neuronal promoter such as, e.g., the MAP2 promoter. For example, in some embodiments, the marker gene is an antibiotic resistance gene, and neuronal cells may be purified by exposing the cell culture to an antibiotic, thus killing cells that have not differentiated into neuronal cells. For example, cells expressing a neomycin gene under the control of the MAP2 promoter may be exposed to G418 to kill non-neuronal cells. Additional methods that may be used with the present invention are described in U.S. patent application Ser. No. 14/664,245, which is incorporated by reference herein without disclaimer in its entirety.

In some embodiments, a population of cells comprising dopaminergic neurons may be purified by exposing the cells to a mitotic inhibitor or chemotherapeutic to kill dividing cells. For example, in some embodiments, a population of cells comprising immature midbrain DA neurons (e.g., D27-D31 cells) produced by methods of the present invention can be purified, e.g., by contacting the cells with Mitomycin C to kill dividing cells.

### VII. USE OF DOPAMINERGIC NEURONS AND DOPAMINERGIC NEURONAL PRECURSORS

The mDA precursor cells provided herein (e.g., D17 cells) can be used in a variety of applications. These methods include but are not limited to: transplantation or implantation of the cells in vivo; screening cytotoxic compounds, carcinogens, mutagens growth/regulatory factors, pharmaceutical compounds, etc., in vitro; elucidating mechanisms of neurodegeneration; studying the mechanism by which drugs and/or growth factors operate; a gene therapy; and the production of biologically active products.

### A. Test Compound Screening

Midbrain DA precursors (e.g., D17 cells) provided herein can be used to screen for factors (such as solvents, small molecule drugs, peptides, and polynucleotides) or environmental conditions (such as culture conditions or manipulation) that affect the characteristics of DA neurons or mDA precursor cells provided herein.

In some applications, stem cells (differentiated or undifferentiated) are used to screen factors that promote maturation of cells along the neural lineage, or that promote proliferation and maintenance of such cells in long-term culture. For example, candidate neural maturation factors or growth factors can be tested by adding them to stem cells in different wells, and then determining any phenotypic change that results, according to desirable criteria for further culture and use of the cells.

Screening applications of the present disclosure include the testing of pharmaceutical compounds in drug research. Standard methods of testing are provided, e.g., in In vitro Methods in Pharmaceutical Research, Academic Press, 1997). In certain aspects of the embodiments, cells produced by methods detailed herein may be used as test cells for standard drug screening and toxicity assays (e.g., to identify, confirm, and test for specification of function or for testing delivery of therapeutic molecules to treat cell lineage specific disease), as have been previously performed on primary neurons in short-term culture. Assessment of the activity of candidate pharmaceutical compounds generally involves combining the neurons provided in certain aspects of this invention with the candidate compound, determining any change in the electrophysiology, morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on neurons cells, or because a compound designed to have effects elsewhere may have unintended neural side effects. Two or more drugs can be tested in combination (by combining with the cells either simultaneously or sequentially), to detect possible drug-drug interaction effects.

In some applications, compounds can be screened or tested for potential neurotoxicity. Cytotoxicity can be determined in the first instance by the effect on cell viability, survival, morphology, or leakage of enzymes into the culture medium. In some embodiments, testing is performed to determine whether the compound(s) affect cell function (such as neurotransmission or electrophysiology) without causing toxicity.

### B. Treatment of Diseases of the Central Nervous System

### 1. Disease of the Central Nervous System

Dopaminergic neurons and mDA precursor cells (e.g., D17 cells) provided herein can be transplanted to regenerate neural cells in an individual having a disease of the central nervous system (CNS). In some embodiments, mDA precursor cells produced according to methods of the present invention may be administered to a subject to treat a CNS disease (e.g., administered to the brain or midbrain, such as the caudate nucleus, putamen, or substantia nigra to treat Parkinson's Disease). Such diseases can include, but are not limited to, neurodegenerative diseases, such as parkinsonism.

As used herein, term “parkinsonism” refers to a group of diseases that are all linked to an insufficiency of dopamine in the basal ganglia which is a part of the brain that controls movement. Symptoms include tremor, bradykinesia (extreme slowness of movement), flexed posture, postural instability, and rigidity. A diagnosis of parkinsonism requires the presence of at least two of these symptoms, one of which must be tremor or bradykinesia. The most common form of parkinsonism is idiopathic, or classic, Parkinson's disease (PD), but for a significant minority of diagnoses, about 15 percent of the total, one of the Parkinson's plus syndromes (PPS) may be present. These syndromes also known as atypical parkinsonism, include corticobasal degeneration, Lewy body dementia, multiple systematrophy, and progressive supranuclear palsy. In general, Parkinson's disease involves the malfunction and death of vital nerve cells in the brain primarily in an area of the brain called the substantia nigra. Many of these vital nerve cells make dopamine. When these neurons die off, the amount of dopamine decreases, leaving a person unable to control movement normally. The intestines also have dopamine cells that degenerate in Parkinson's disease patients, and this may be an important causative factor in the gastrointestinal symptoms that are part of the disease. The particular symptoms that an individual experiences can vary from person to person. Primary motor signs of Parkinson's disease include the following: tremor of the hands, arms, legs, jaw and face, bradykinesia or slowness of movement, rigidity or stiffness of the limbs and trunk and postural instability or impaired balance and coordination.

In some embodiments, iPSC-derived mDA precursor cells (e.g., D17 cells) can exhibit improved properties for clinical treatment of PD as compared to other iPSC-derived mature mDA neurons. iPSC-derived mDA neurons differentiated via a floor plate intermediate, may engraft, survive long-term, and reduce or reverse drug-induced motor asymmetry in athymic rats with unilateral 6-hydroxydopamine (6-OHDA) lesions (Hiller et al., 2020; Wakeman et al., 2017). Cells in various stages of development have been transplanted previously (Bye, Thompson, & Parish, 2012; Kirkeby et al., 2012; Kriks et al., 2011; Niclis et al., 2017).

In some embodiments, mDA precursor cells provided herein can display superior properties for clinical treatment of diseases such as PD. As shown in the below Examples, 1) a line of iPSCs and a differentiation process leading to the generation of mDA precursor cells that can be used clinically was developed; 2) intrastriatal grafts of iPSC-derived mDA progenitors (cryopreserved on day 17 in vitro) in immunocompromised rats completely reversed 6-OHDA-induced motor asymmetry, survive in large numbers and densely reinnervate the host striatum, and are superior to grafts of cells cryopreserved on days 24 and 37; 3) that D17 progenitors were observed to mature and maintain the appropriate mDA lineage in vivo; 4) that D17 and D24 grafts placed in the substantia nigra exhibited long-range axonal growth to multiple host targets normally innervated by the mesotelencephalic dopamine system; 5) higher doses of D17 progenitors provided faster and more complete functional recovery than lower doses with corresponding increases in cell survival and graft-derived TH innervation; and 6) neither teratomas nor excessive proliferation of cells were observed when transplanting iPSC subjected to our differentiation protocol. mDA precursor cells provided herein may exhibit one or more of, or all of the above advantages listed above when used clinically.

In some embodiments, the mDA precursor cells (e.g., D17 cells) are administered to a patient to treat a brain disease or a brain injury involving the death of dopaminergic neurons such as, e.g., Parkinson's disease (PD). As shown in the below examples, the mDA precursor cells were observed to engraftment, innervation, and functional efficacy in vivo using an animal model of PD (i.e., hemiparkinsonian rats). mDA progenitor or precursor cells (cryopreserved on Day 17, “D17”), immature mDA neurons (“D24”), and purified mDA neurons (“D37”), were tested and compared to R&D grade purified mDA neurons (D38, “G418”) that are available commercially (Hiller et al., 2020; Wakeman et al., 2017). The D17 or D24 cells were observed to provide long-distance innervation when grafted into the substantia nigra (SN). D17 mDA progenitors were observed to have the most robust survival and fiber outgrowth, and a dose-ranging experiments were used to determine the lowest dose that exerted an early onset of functional recovery in hemiparkinsonian rats. These results demonstrate that the mDA precursor cells provided herein can be used to treat PD in a mammalian subject such as a human.

It is anticipated that a variety of dosages of mDA precursor cells (e.g., D17 cells) as disclosed herein can be therapeutically administered to a mammalian subject such as a human. For example from about 2,500 cells/μL to about 150,000 cells/μL, from about 10,000 cells/μL to about 150,000 cells/μL, from about 40,000 cells/μL to about 100,000 cells/μL, from about 15,000 cells/μL to about 45,000 cells/μL, about 3e6-9e6 cells, 2500, 3000, 4000, 5000, 6000, 7000, 8000, 9000, 1e4, 2e4, 3e4, 4e4, 5e4, 6e4, 7e4, 8e4, 9e4, 1e5, 1.1e5, 1.2e5, 1.3e5, 1.4e5, or 1.5e5 cells/μL midbrain dopaminergic neuronal precursor cells, or any range derivable therein, can be administered to a mammalian subject such as a human. It is anticipated that the total number of cells administered to a mammalian subject such as a human patient may range from about 1e5 to about 100e6, and the total number of cells may be selected by the clinician based on the symptoms and other characteristics of the subject. Preferably, the cells are administered to the brain of the subject. For example, the mDA precursor cells may be administered to the striatum, such as the putamen or substantia nigra, of the subject. In some instances, it may be sufficient to administer the mDA precursor cells at one location in the brain of the subject. In other embodiments, the mDA precursor cells are administered at multiple sites and/or at multiple needle tracts into brain (e.g., the striatum or putamen) of the subject. In human subjects, it is anticipated that administration of the mDA cells at multiple sites in the striatum may in some instances facilitate more extensive innervation by the mDA precursor cells.

### 2. Methods for Administering Cells

The cells provided herein can be administered to a subject either locally or systemically. In some preferred embodiments mDA precursor cells (e.g., D17 cells) are administered into the brain of a subject. Methods for administering DA neurons to a subject, such as stereotaxic administration to the brain, are known in the art and can be applied to the cells and cell cultures provided herein. If the patient is receiving cells derived from his or her own cells, this is called an autologous transplant; such a transplant has little likelihood of rejection.

Exemplary methods of administering stem cells or differentiated neuronal cells to a subject, particularly a human subject, include injection or transplantation of the cells into target sites (e.g., striatum and/or substantia nigra) in the subject. The mDA precursor cells can be inserted into a delivery device which facilitates introduction, by injection or transplantation, of the cells into the subject. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. The stem cells can be inserted into such a delivery device, e.g., a syringe, in different forms. For example, the cells can be suspended in a solution, be in cell aggregates, or alternatively embedded in a support matrix when contained in such a delivery device.

Support matrices in which the stem cells, neurons, or neuronal precursor cells can be incorporated or embedded include matrices that are recipient-compatible and that degrade into products that are not harmful to the recipient. The support matrices can be natural (e.g., hyaluronic acid, collagen, etc.) and/or synthetic biodegradable matrices. Synthetic biodegradable matrices that may be used include synthetic polymers such as polyanhydrides, polyorthoesters, and polylactic acid. In some embodiments, dopaminergic neurons (e.g., dopaminergic neurons that are not fully differentiated) are embedded in hyaluronic acid matrix and administered to a subject to treat a neurodegenerative disease (e.g., Parkinson's disease).

As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is known in the art. The solution is preferably sterile and fluid to the extent that easy syringability exists.

Preferably, the solution is stable under the conditions of manufacture and storage and preserved against the contaminating action of microorganisms such as bacteria and fungi. In some embodiments a solution containing mDA precursor cells (e.g., D17 cells) is administered to a patient in sterile solution of BSS PLUS (Alcon, Fort Worth, TX). If desired a preservative or antibiotic may be included in the pharmaceutical composition for administration. Solutions of the invention can be prepared by incorporating mDA neuronal precursor cells as described herein in a pharmaceutically acceptable carrier or diluent and, other ingredients if desired.

### 3. Dosage and Administration

In one aspect, the methods described herein provide a method for enhancing engraftment of neuronal progenitor cells (e.g., D17 cells) or DA neurons in a subject. In some embodiments, the subject is a mammal, such as a human.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of each active ingredient required to be administered depend on the judgment of the practitioner and may be particular to each patient or subject. Suitable dosage ranges may depend on the route of administration, and various methods of administration can be used.

A variety of dosages of mDA precursor cells (e.g., D17 cells) as disclosed herein can be therapeutically administered to a mammalian subject. For example from about 2,500 cells/μL to about 150,000 cells/μL, from about 10,000 cells/μL to about 150,000 cells/μL, from about 40,000 cells/μL to about 100,000 cells/μL, about 15,000-45000 cells/μL, about 1e6-9e6 cells/μL, about 2500, 3000, 4000, 5000, 6000, 7000, 8000, 9000, 1e4, 2e4, 3e4, 4e4, 5e4, 6e4, 7e4, 8e4, 9e4, 1e5, 1.1e5, 1.2e5, 1.3e5, 1.4e5, or 1.5e5 midbrain dopaminergic neuronal precursor cells, or any range derivable therein, can be administered to a mammalian subject such as a human. It is anticipated that the total number of cells administered to a mammalian subject such as a human patient may range from about 1e5 to about 100e6, and the total number of cells may be selected by the clinician based on the symptoms and other characteristics of the subject. In some embodiments, the mDA precursor cells are administered to the brain or central nervous system of a mammalian subject, preferably a human patient, via injection (e.g., at a single site or at multiple sites in the brain, such as into the striatum or putamen).

### 4. Efficacy

The efficacy of a given treatment to enhance DA neuron engraftment can be determined by the skilled artisan. However, a treatment is considered “effective treatment,” as the term is used herein, if any one or all of the signs or symptoms of e.g., poor DA neuron engraftment are altered in a beneficial manner, other clinically accepted symptoms are improved, or even ameliorated, e.g., by at least 10% following treatment with a cell population as described herein. Efficacy can also be measured by a failure of an individual to worsen as assessed by hospitalization, need for medical interventions (i.e., progression of the disease is halted), or incidence of engraftment failure. Methods of measuring these indicators are known to those of skill in the art and/or are described herein. Treatment includes any treatment of a disease in an individual or an animal (some non-limiting examples include a human or a mammal) and includes: (1) inhibiting the disease, e.g., preventing engraftment failure; or (2) relieving the disease, e.g., causing regression of one or more symptoms. An effective amount for the treatment of a disease means an amount which, when administered to a mammal in need thereof, is sufficient to result in a treatment or therapeutic benefit for that disease. Efficacy of an agent can be determined by assessing physical indicators of, for example, DA neuron engraftment, such as, e.g., tremor, bradykinesia, flexed posture, balance and coordination, etc. In some embodiments, engraftment or neural function may be measured in vivo (e.g., in humans) using a PET scan to detect metabolism, activity, dopaminergic neurotransmission (e.g., using PET tracers for imaging of the dopaminergic system). Efficacy can be assessed in animal models of Parkinson's disease, for example, by performing behavioral tests, such as step tests or cylinder tests.

### C. Distribution for Commercial, Therapeutic, and Research Purposes

For purposes of manufacture, distribution, and use, the neural cells such as midbrain DA neuronal precursor cells as described herein may be supplied in the form of a cell culture or suspension in an isotonic excipient or culture medium, optionally frozen to facilitate transportation or storage.

mDA precursor cells described herein may be provided using different reagent systems, e.g., comprising a set or combination of cells that exist at any time during manufacture, distribution, or use. The cell sets may comprise any combination of two or more cell populations described in this disclosure, exemplified but not limited to programming-derived cells (neural lineage cells, their precursors and subtypes), in combination with undifferentiated stem cells or other differentiated cell types. The cell populations in the set may share the same genome or a genetically modified form thereof. Each cell type in the set may be packaged together, or in separate containers in the same facility, or at different locations, at the same or different times, under control of the same entity or different entities sharing a business relationship.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods for Generating Cell Cultures

Midbrain neuronal differentiation of human induced pluripotent stem (iPS) cell lines expanded on VTN-TN in Essential 8 medium was performed with small molecule and growth factor induction using a variety of differentiation media compositions and schedules as detailed in Table 1. Generally, the iPS cells were cultured in D1 DA Neuron Induction Medium on Day 1, D2 Neuron Induction Medium on Day 2, and D3-D4 DA Induction Medium on Day 3 and 4. On Day 5, the cells were dissociated with TrypLE for 15 minutes and collected in DA Quench Medium before transferring the cells to a spinner flask suspension culture to form aggregates in D5 DA Neuron Aggregate Formation Medium.

On Day 6, the aggregates were settled, about 66% of the medium was removed, and the aggregates were fed DA Neuron Induction Medium. On Days 7-16, the aggregates were fed daily with DA Neuron Aggregate Maintenance Medium, and the medium was changed on Day 11 through 16. On Day 17, aggregates were dissociated to a single-cell suspension with TrypLE and plated onto Matrigel in D17 DA Neuron Aggregate Plating Medium. On Days 18, 20, 22 the medium was replaced with DopaNeuron Maturation Medium. On Day 24, the cells were dissociated using Accutase and plated in DA Neuron Maturation Plating Medium. The next day, the medium was replaced with DopaNeuron Maturation Medium.

On Days 27 and 29, the media was replaced with DA Neuron Maturation Medium plus Mitomycin C. On Day 31, the cells were dissociated with Accutase and re-plated onto poly-L-ornithine (PLO)/Laminin-coated flasks in DA Neuron Maturation Plating Medium. Next, on Days 32, 34, and 36, the cells were fed DopaNeuron Maturation Medium. On Day 37 or 38, the cells were again dissociated with Accutase and subjected to analysis or cryopreserved for later use.

TABLE 1
Regular timing media conditions (200 nM LDN).

| | | | | |
|---|---|---|---|---|
| Component | Vendor | Cat# | Stock | Final Conc. |

E8 Plating Medium (Day −2)

| | | | | |
|---|---|---|---|---|
| Essential 8 Basal Medium | Life Technologies | A14666SA | 1× | 98% |
| Essential 8 Supplement | Life Technologies | A14666SA | 50× | 2% |
| H1152 | CDI | H024 | 100 μM | 1 μM |

E8 Medium (Days −1 and 0)

| | | | | |
|---|---|---|---|---|
| Essential 8 Basal Medium | Life Technologies | A14666SA | 1× | 98% |
| Essential 8 Supplement | Life Technologies | A14666SA | 50× | 2% |

D1 DA Induction Medium (Day 1)

| | | | | |
|---|---|---|---|---|
| DMEM/F12 | Life Technologies | 11330-032 | 1× | 98% |
| B-27 Supplement (+VitA) | Life Technologies | 17504-044 | 2× | 2% |
| LDN-193189 | Stemgent | 04-0074 | 10 mM | 200 nM |
| Purmorphamine | Cayman | 10009634 | 10 mM | 2 μM |
| C25II SHH | R&D Systems | 464-SH | 100 μg/mL | 100 ng/ml |
| CHIR99021 | Stemgent | 04-0004 | 20 mM | 1.75 μM |

D2 DA Induction Medium (Day 2)

| | | | | |
|---|---|---|---|---|
| DMEM/F12 | Life Technologies | 11330-032 | 1× | 98% |
| B-27 Supplement (+VitA) | Life Technologies | 17504-044 | 2× | 2% |
| LDN-193189 | Stemgent | 04-0074 | 10 mM | 200 nM |
| Purmorphamine | Cayman | 10009634 | 10 mM | 2 uM |
| C25II SHH | R&D Systems | 464-SH | 100 μg/mL | 100 ng/ml |
| CHIR99021 | Stemgent | 04-0004 | 20 mM | 1.25 μM |
| PD0325901 | Stemgent | 04-0006 | 10 mM | 1.0 μM |

D3-4 DA Induction Medium (Days 3 and 4)

| | | | | |
|---|---|---|---|---|
| DMEM/F12 | Life Technologies | 11330-032 | 1× | 98% |
| B-27 Supplement (−VitA) | Life Technologies | 12587-010 | 2× | 2% |
| LDN-193189 | Stemgent | 04-0074 | 10 mM | 200 nM |
| Purmorphamine | Cayman | 10009634 | 10 mM | 2 μM |
| C25II SHH | R&D Systems | 464-SH | 100 μg/ml | 100 ng/ml |
| CHIR99021 | Stemgent | 04-0004 | 20 mM | 1.25 μM |
| PD0325901 | Stemgent | 04-0006 | 10 mM | 1.0 μM |

DA Quench Medium 1 (Days 5 and 17)

| | | | | |
|---|---|---|---|---|
| DMEM/F12 | Life Technologies | 11330-032 | 1× | 98% |
| B-27 Supplement (−VitA) | Life Technologies | 12587-010 | 2× | 2% |
| Blebbistatin | Sigma | B0560 | 10,000× | 2.5 μM |

D5 DA Aggregate Formation Medium (Day 5)

| | | | | |
|---|---|---|---|---|
| DMEM/F12 | Life Technologies | 11330-032 | 1× | 98% |
| B-27 Supplement (−VitA) | Life Technologies | 12587-010 | 2× | 2% |
| LDN-193189 | Stemgent | 04-0074 | 10 mM | 200 nM |
| Purmorphamine | Cayman | 10009634 | 10 mM | 2 μM |
| C25II SHH | R&D Systems | 464-SH | 100 μg/ml | 100 ng/ml |
| CHIR99021 | Stemgent | 04-0004 | 20 mM | 1.25 μM |
| Blebbistatin | Sigma | B0560 | 2,500× | 10 μM |

D6 DA Induction Medium (Day 6)

| | | | | |
|---|---|---|---|---|
| DMEM/F12 | Life Technologies | 11330-032 | 1× | 98% |
| B-27 Supplement (−VitA) | Life Technologies | 12587-010 | 2× | 2% |
| LDN-193189 | Stemgent | 04-0074 | 10 mM | 200 nM |
| Purmorphamine | Cayman | 10009634 | 10 mM | 2 μM |
| C25II SHH | R&D Systems | 464-SH | 100 μg/mL | 100 ng/ml |
| CHIR99021 | Stemgent | 04-0004 | 20 mM | 1.25 μM |

D7-10 DA Aggregate Maintenance Medium (Days 7-10)

| | | | | |
|---|---|---|---|---|
| DMEM/F12 | Life Technologies | 11330-032 | 1× | 98% |
| B-27 Supplement (−VitA) | Life Technologies | 12587-010 | 2× | 2% |
| LDN-193189 | Stemgent | 04-0074 | 10 mM | 200 nM |
| CHIR99021 | Stemgent | 04-0004 | 20 mM | 1.25 μM |

D11-16 DA Aggregate Maintenance Medium (Days 11-16)

| | | | | |
|---|---|---|---|---|
| DMEM/F12 | Life Technologies | 11330-032 | 1× | 98% |
| B-27 Supplement (−VitA) | Life Technologies | 12587-010 | 2× | 2% |
| LDN-193189 | Stemgent | 04-0074 | 10 mM | 200 nM |
| CHIR99021 | Stemgent | 04-0004 | 20 mM | 3.0 μM |
| FGF8 | R&D Systems | 423-F8 | 50 μg/mL | 100 ng/ml |

D17 DA Aggregate Plating Medium (Days 17)

| | | | | |
|---|---|---|---|---|
| DMEM/F12 | Life Technologies | 11330-032 | 1× | 98% |
| B-27 Supplement (−VitA) | Life Technologies | 12587-010 | 2× | 2% |
| LDN-193189 | Stemgent | 04-0074 | 10 mM | 200 nM |
| CHIR99021 | Stemgent | 04-0004 | 20 mM | 3.0 μM |
| FGF8 | R&D Systems | 423-F8 | 50 μg/mL | 100 ng/ml |
| Blebbistatin | Sigma | B0560 | 10,000× | 2.5 μM |

DA Maturation Medium (Days 18+)

| | | | | |
|---|---|---|---|---|
| Neurobasal | Life Technologies | 21103-049 | 1× | 98% |
| Glutamax | Life Technologies | 35050-061 | 200 mM | 2 mM |
| B-27 Supplement (−VitA) | Life Technologies | 12587-010 | 2× | 2% |
| Ascorbic Acid | Sigma | A4403 | 200 mM | 200 μM |
| Rec Hu BDNF | R&D Systems | 248-BD | 20 μg/mL | 20 ng/ml |
| Rec Hu GDNF | R&D Systems | 212-GD | 20 μg/mL | 20 ng/ml |
| Rec Hu TGFB3 | R&D Systems | 243-B3 | 10 μg/mL | 1 ng/ml |
| dbcAMP | Sigma | D0627 | 100 mM | 500 μM |
| DAPT | Sigma | D5942 | 20 mM | 5 μM |

DA Maturation Medium + MMC (Days 27 and 29)

| | | | | |
|---|---|---|---|---|
| Neurobasal | Life Technologies | 21103-049 | 1× | 98% |
| Glutamax | Life Technologies | 35050-061 | 200 mM | 2 mM |
| B-27 Supplement (−VitA) | Life Technologies | 12587-010 | 2× | 2% |
| Ascorbic Acid | Sigma | A4403 | 200 mM | 200 μM |
| Rec Hu BDNF | R&D Systems | 248-BD | 20 μg/mL | 20 ng/ml |
| Rec Hu GDNF | R&D Systems | 212-GD | 20 μg/mL | 20 ng/ml |
| Rec Hu TGFB3 | R&D Systems | 243-B3 | 10 μg/mL | 1 ng/mL |
| dbcAMP | Sigma | D0627 | 100 mM | 500 μM |
| DAPT | Sigma | D5942 | 20 mM | 5 μM |
| Mitomycin C | Sigma | M4287 | 1 mg/ml | 100 ng/ml |

DA Quench Medium 2 (Days 18+)

| | | | | |
|---|---|---|---|---|
| Neurobasal | Life Technologies | 21103-049 | 1× | 98% |
| Glutamax | Life Technologies | 35050-061 | 200 mM | 2 mM |
| B-27 Supplement (−VitA) | Life Technologies | 12587-010 | 2× | 2% |
| Blebbistatin | Sigma | B0560 | 10,000× | 2.5 μM |

DA Maturation Plating Medium (Days 18+)

| | | | | |
|---|---|---|---|---|
| Neurobasal | Life Technologies | 21103-049 | 1× | 98% |
| Glutamax | Life Technologies | 35050-061 | 200 mM | 2 mM |
| B-27 Supplement (−VitA) | Life Technologies | 12587-010 | 2× | 2% |
| Ascorbic Acid | Sigma | A4403 | 200 mM | 200 μM |
| Rec Hu BDNF | R&D Systems | 248-BD | 20 μg/mL | 20 ng/ml |
| Rec Hu GDNF | R&D Systems | 212-GD | 20 μg/mL | 20 ng/ml |
| Rec Hu TGFB3 | R&D Systems | 243-B3 | 10 μg/mL | 1 ng/ml |
| dbcAMP | Sigma | D0627 | 100 mM | 500 μM |
| DAPT | Sigma | D5942 | 20 mM | 5 μM |
| Blebbistatin | Sigma | B0560 | 10,000× | 2.5 μM |

Midbrain neuronal differentiation of human induced pluripotent stem (iPS) cell lines expanded on VTN-TN in Essential 8 medium was performed with small molecule and growth factor induction using a variety of differentiation media compositions and schedules as detailed in Table 2. Generally, the iPS cells were cultured in D1 DA Neuron Induction Medium on Day 1, D2 Neuron Induction Medium on Day 2, and D3-D4 DA Induction Medium on Day 3 and 4. On Day 5, the cells were dissociated with TrypLE for 15 minutes and collected in DA Quench Medium before transferring the cells to a spinner flask suspension culture to form aggregates in D5 DA Neuron Aggregate Formation Medium.

On Days 6 and 7, the aggregates were settled, about 66% of the medium was removed, and the aggregates were fed D6-7 DA Induction Medium (Day 6-7). On Days 8-10, the aggregates were fed daily with D8-10 DA Aggregate Maintenance Medium. On days 11-16 the aggregates were fed daily with D11-16 DA Aggregate Maintenance Medium. On Day 17, aggregates were dissociated to a single-cell suspension with TrypLE and allowed to sit in quench (DA Quench Medium 1) for 15 min before washing and cryopreserving in cryopreservation medium. Cryopreserved cells were stored in the vapor phase of liquid nitrogen.

TABLE 2
FCDI DAPC-1

| | | | | |
|---|---|---|---|---|
| Component | Vendor | Cat# | Stock | Final Conc. |

E8 Plating Medium (Day −2)

| | | | | |
|---|---|---|---|---|
| Essential 8 Basal Medium | Life | A14666SA | 1× | 98% |
| | Technologies | | | |
| Essential 8 Supplement | Life | A14666SA | 50× | 2% |
| | Technologies | | | |
| H1152 | FCDI | H024 | 100 μM | 1 μM |

E8 Medium (Days −1 and 0)

| | | | | |
|---|---|---|---|---|
| Essential 8 Basal Medium | Life | A14666SA | 1× | 98% |
| | Technologies | | | |
| Essential 8 Supplement | Life | A14666SA | 50× | 2% |
| | Technologies | | | |

D1 DA Induction Medium (Day 1)

| | | | | |
|---|---|---|---|---|
| DMEM/F12 | Life | 11330-032 | 1× | 98% |
| | Technologies | | | |
| B-27 Supplement (+VitA) | Life | 17504-044 | 2× | 2% |
| | Technologies | | | |
| LDN-193189 | Stemgent | 04-0074 | 10 mM | 200 nM |
| Purmorphamine | Cayman | 10009634 | 10 mM | 2 μM |
| Recombinant Human Shh (C24II), | R&D Systems | 1845-GMP | 100 μg/ml | 100 ng/ml |
| GMP | | | | |

D2 DA Induction Medium (Day 2)

| | | | | |
|---|---|---|---|---|
| DMEM/F12 | Life | 11330-032 | 1× | 98% |
| | Technologies | | | |
| B-27 Supplement (+VitA) | Life | 17504-044 | 2× | 2% |
| | Technologies | | | |
| LDN-193189 | Stemgent | 04-0074 | 10 mM | 200 nM |
| Purmorphamine | Cayman | 10009634 | 10 mM | 2 μM |
| Recombinant Human Shh (C24II), | R&D Systems | 1845-GMP | 100 μg/mL | 100 ng/ml |
| GMP | | | | |
| CHIR99021 | Stemgent | 04-0004 | 20 mM | 1.65 μM |

D3-4 DA Induction Medium (Days 3 and 4)

| | | | | |
|---|---|---|---|---|
| DMEM/F12 | Life | 11330-032 | 1× | 98% |
| | Technologies | | | |
| B-27 Supplement (−VitA) | Life | 12587-010 | 2× | 2% |
| | Technologies | | | |
| LDN-193189 | Stemgent | 04-0074 | 10 mM | 200 nM |
| Purmorphamine | Cayman | 10009634 | 10 mM | 2 μM |
| Recombinant Human Shh (C24II), | R&D Systems | 1845-GMP | 100 μg/mL | 100 ng/ml |
| GMP | | | | |
| CHIR99021 | Stemgent | 04-0004 | 20 mM | 1.65 μM |
| PD0325901 | Stemgent | 04-0006 | 10 mM | 1.0 μM |

DA Quench Medium 1 (Days 5 and 17)

| | | | | |
|---|---|---|---|---|
| DMEM/F12 | Life | 11330-032 | 1× | 98% |
| | Technologies | | | |
| B-27 Supplement (−VitA) | Life | 12587-010 | 2× | 2% |
| | Technologies | | | |
| Blebbistatin | Sigma | B0560 | 10,000× | 2.5 μM |
| Benzonase | MilliporeSigma | 101697 | | 15 Units/mL |

D5 DA Aggregate Formation Medium (Day 5)

| | | | | |
|---|---|---|---|---|
| DMEM/F12 | Life | 11330-032 | 1× | 98% |
| | Technologies | | | |
| B-27 Supplement (−VitA) | Life | 12587-010 | 2× | 2% |
| | Technologies | | | |
| LDN-193189 | Stemgent | 04-0074 | 10 mM | 200 nM |
| Purmorphamine | Cayman | 10009634 | 10 mM | 2 μM |
| Recombinant Human Shh (C24II), | R&D Systems | 1845-GMP | 100 μg/mL | 100 ng/ml |
| GMP | | | | |
| CHIR99021 | Stemgent | 04-0004 | 20 mM | 1.65 μM |
| PD0325901 | Stemgent | 04-0006 | 10 mM | 1.0 μM |
| Blebbistatin | Sigma | B0560 | 2,500× | 10 μM |

D6-7 DA Induction Medium (Day 6-7)

| | | | | |
|---|---|---|---|---|
| DMEM/F12 | Life | 11330-032 | 1× | 98% |
| | Technologies | | | |
| B-27 Supplement (−VitA) | Life | 12587-010 | 2× | 2% |
| | Technologies | | | |
| LDN-193189 | Stemgent | 04-0074 | 10 mM | 200 nM |
| Purmorphamine | Cayman | 10009634 | 10 mM | 2 μM |
| Recombinant Human Shh (C24II), | R&D Systems | 1845-GMP | 100 μg/mL | 100 ng/mL |
| GMP | | | | |
| CHIR99021 | Stemgent | 04-0004 | 20 mM | 1.65 μM |

D8-10 DA Aggregate Maintenance Medium (Days 8-10)

| | | | | |
|---|---|---|---|---|
| DMEM/F12 | Life | 11330-032 | 1× | 98% |
| | Technologies | | | |
| B-27 Supplement (−VitA) | Life | 12587-010 | 2× | 2% |
| | Technologies | | | |
| LDN-193189 | Stemgent | 04-0074 | 10 mM | 200 nM |
| CHIR99021 | Stemgent | 04-0004 | 20 mM | 1.65 μM |

D11-16 DA Aggregate Maintenance Medium (Days 11-16)

| | | | | |
|---|---|---|---|---|
| DMEM/F12 | Life | 11330-032 | 1× | 98% |
| | Technologies | | | |
| B-27 Supplement (−VitA) | Life | 12587-010 | 2× | 2% |
| | Technologies | | | |
| LDN-193189 | Stemgent | 04-0074 | 10 mM | 200 nM |
| CHIR99021 | Stemgent | 04-0004 | 20 mM | 3.0 μM |
| FGF8 | R&D Systems | 423-F8 | 50 μg/ml | 100 ng/ml |

Cryopreservation (Day 17)

| | | | | |
|---|---|---|---|---|
| CryoStor CS10 | Biolife | 210102 | 1× | 100% |
| | Solutions | | | |

### Example 2

### mDA Progenitor Patterning

Efficient patterning of mDA progenitors, as measured by the percentage of cells co-expressing PoxA2 and Lmx1 on process day 17, is generally required for obtaining a highly enriched population of mDA neurons at the end of the manufacturing process. If the majority of the cells on day 17 are not mDA progenitors, the neurons obtained will have a large population of non-midbrain phenotype neurons, or will have an outgrowth of proliferative cells that typically leads to neuron detachment or difficulties or an inability to purify the post-mitotic neurons.

These mono-SMAD experiments were repeated, with the modification that Benzonase® (endonuclease, EMD Millipore) was included in the incubation on Day 5 at a concentration of 10 U/mL. Inclusion of the Benzonase® in the incubation on Day 5 was observed to reduce or prevent excessive clumping in the aggregate formation.

### Example 3

### Flow Cytometry Assay for FoxA2/Lmx1 Co-Expression

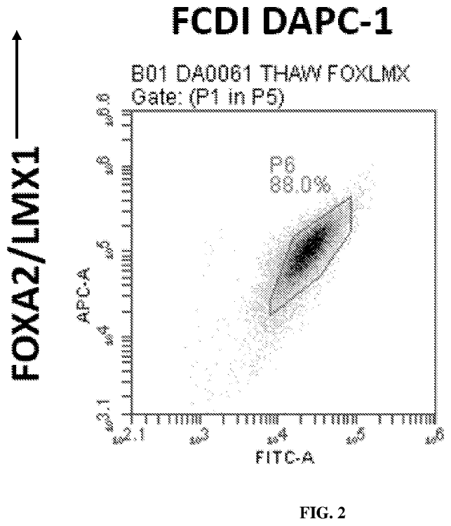

FoxA2/Lmx1 co-expression is a critical readout for successful dopamine neuron progenitor patterning, and therefore an intracellular flow cytometry assay was developed that is less subjective and variable than results derived using cell counting software run on immunocytochemistry images. The assay can accurately quantify the percentage of cells co-expressing FoxA2 and Lmx1 on process day 17 to day 24, with results that correlate to counts from analyzed ICC images. Progenitor patterning is considered successful when the cells are >65% FoxA2+/Lmx1+ on day 17 (FIG. 2).

### Example 4

Dopamine Release from iPSC-mDA Neurons

iPSC line “K” (21534.101) was differentiated to process completion (day 37) and cryopreserved. Cells were thawed and plated at high density (8.8×10⁵/cm²). The cells were fed with Maturation Medium without DAPT every third day for a total of 14 days. On the assay day, cells were washed and incubated 30 min with HBSS (with or without 56 mM KCl). The dopamine concentration in the release solution was determined using a competitive dopamine ELISA kit (Eagle Biosciences). No dopamine release was detected from iPSC-derived forebrain neurons (iCell Neurons). Conversely, iPSC-mDA cells derived using the optimized mono-SMADi process (DA Therapy Neurons) secreted at least as much dopamine as cells derived using the optimized dual-SMAD process (iCell DopaNeurons). Thus, the cells are able to perform a key functional attribute of mature dopamine neurons.

### Example 5

Electrical Activity of iPSC-mDA Neurons

Cryopreserved iPSC-mDA neurons were thawed and plated onto PEI-coated 48-well multielectrode array (MEA) plates. Cells were cultured according to the FUJIFILM Cellular Dynamics, Inc application protocol “Measuring synchronous neuronal activity on the Maestro multielectrode array” in U.S. application Ser. No. 14/830,162. Neurons made with the optimized mono-SMADi protocol (DA Therapy) demonstrated similar electrical activity compared to cells made with the optimized dual-SMADi protocol (iCell Dopa G100), including mean firing rate (mFR), bursting (macro BPMs) and connectivity. Mean firing rate (mFR), frequency, and connectivity burst intensity increased with time, plateauing by approximately day 16 post-thaw. Temporal Raster plots showed clean inter-spike intervals, high burst intensities, and bursting across all electrodes in a well, demonstrating a high degree of electrical activity.

### Example 6

### Quantitative Gene Expression Profile of FCDI DAPC-1 Neurons

RNA was extracted from four batches of iPSC-mDA cells derived using the optimized mono-SMADi process (Batch 1-4) and one batch of iPSC-mDA cells derived using the optimized dual-SMADi protocol (iCell DopaNeurons) on process day 37. After RNA isolation, real-time quantitative polymerase chain reaction (PCR) was performed using TaqMan Gene Expression Assays (Applied Biosystems), with results expressed as relative expression to GAPDH control. Values <10⁻⁴ are considered background (shaded box). Expression of midbrain and mDA neuron markers were similar between batches and between cells made using the different protocols. Markers for non-midbrain regions or non-mDA cell types were low, and also similar between mono-SMADi and dual-SMADi-derived cells. Results are shown in FIG. 12 and FIG. 13.

### Example 7

Engraftment of iPSC-DA Progenitors and Neurons in Rat Parkinson's Disease Model System

iPSC line “K” was differentiated using the optimized mono-SMADi protocol and cryopreserved at different stages of the differentiation process (Day 17, day 24, and Day 37). In addition, iPSC-mDA cells derived using the optimized dual-SMADi protocol (iCell Dopa) were cryopreserved on process day 37. Cells were thawed and transplanted bilaterally to the striatum (4.5×10⁵ cells/injection) of 6-OHDA-treated but asymptomatic nude (RNU) rats (n=3 per group). After 3 months, engraftment and innervation of the cells was assessed by histology of coronal sections. Although neuron engraftment and innervation was observed in all four groups (Human NCAM stain), the iPSC-DA progenitor cells (day 17) and immature mDA neurons (day 24) had much larger grafts and greater innervation compared to the more mature mono-SMADi and dual-SMADi-derived mDA neurons (day 37 and day 37 iCell Dopa, respectively). In addition, larger numbers of DA neurons (TH+) were observed in the progenitor and immature DA neuron grafts. Ki67 staining revealed almost no proliferative cells in the grafts from day 37 cells, and few Ki67⁺ cells in the grafts from day 17 and day 24 cells. No tumors, neural outgrowth, or other adverse effects were observed in any of these animals. These results suggest that cells drawn from earlier in the optimized mono-SMADi differentiation process (day 17-24) are better able to engraft and innervate compared to more mature cells. Results are shown in FIG. 9.

### Example 8

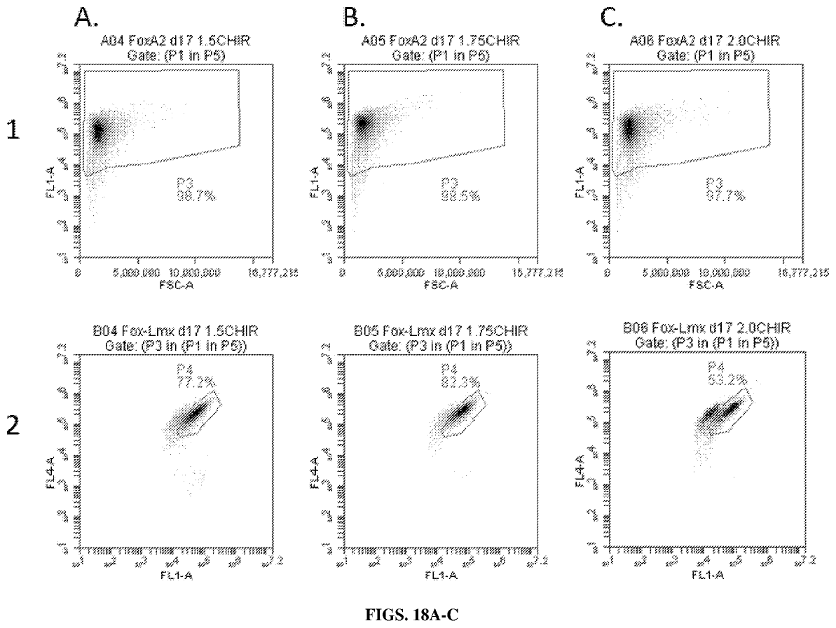

The above experiments were repeated using a variety of CHIR concentrations. Results are shown in FIG. 18. As shown in the results, marked improvements were observed when using CHIR99021 concentrations from about 1.5 to about 1.75 μM.

### Example 9

### Characterization of mDA Progenitor Cells

A cryopreserved single-cell suspension containing iPSC derived midbrain dopamine neuron progenitor cells (“FCDI DAPC-1”) were generated via the methods described in the above Examples. The cells were derived from an allogeneic human iPSC line (FCDI designation 21534.101) via directed differentiation to obtain a population of dopaminergic neuron progenitor cells.

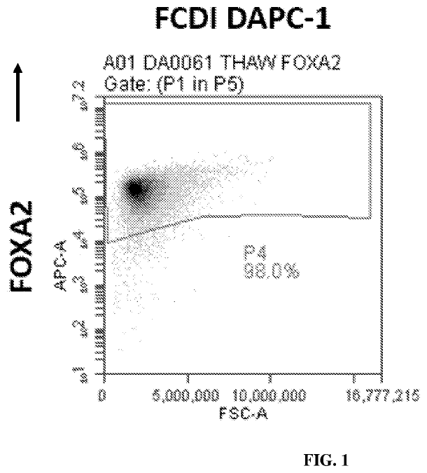

FOXA2 flow cytometry assay was performed on the mDA progenitor cells generated as described in the above Examples. The FOXA2 flow cytometry assay indicated that the mDA progenitor cells showed correct floor plate patterning of FCDI DAPC-1. Results are shown in FIG. 1.

The FOXA2/LMX flow cytometry assay revealed co-expression of FOXA2 and LMX in FCDI DAPC-1 mDA progenitor cells. Parallel ICC staining was performed for comparison, and co-expressing cells appearing yellow were observed. Results are shown in FIG. 2.

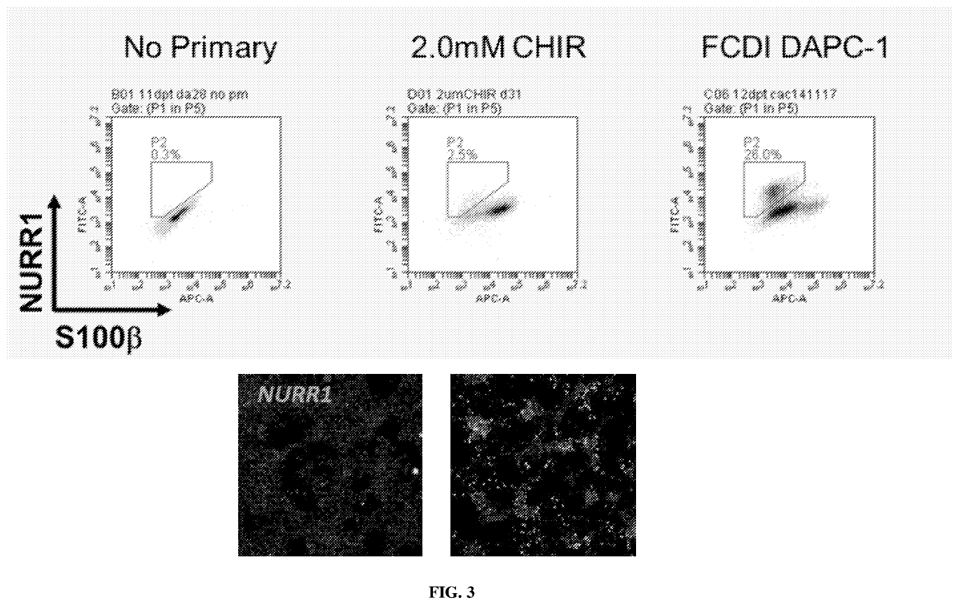

After 12 days in culture post thaw, FCDI DAPC-1 mDA progenitor cells have the potential to differentiate into immature DA neurons as demonstrated by NURR1 expression. Parallel ICC staining was also performed. Results are shown in FIG. 3.

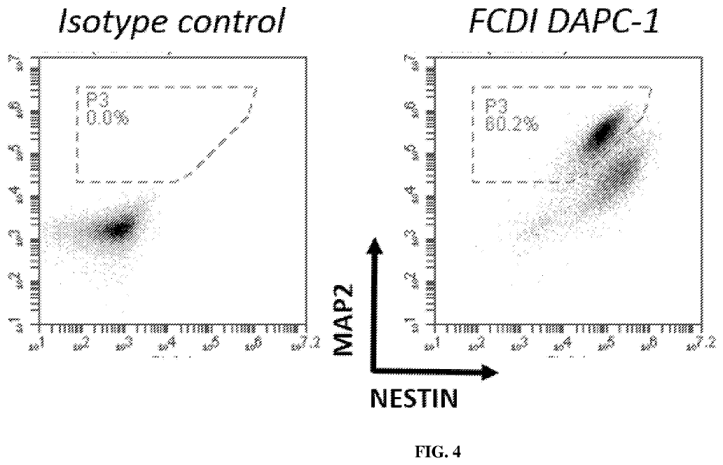

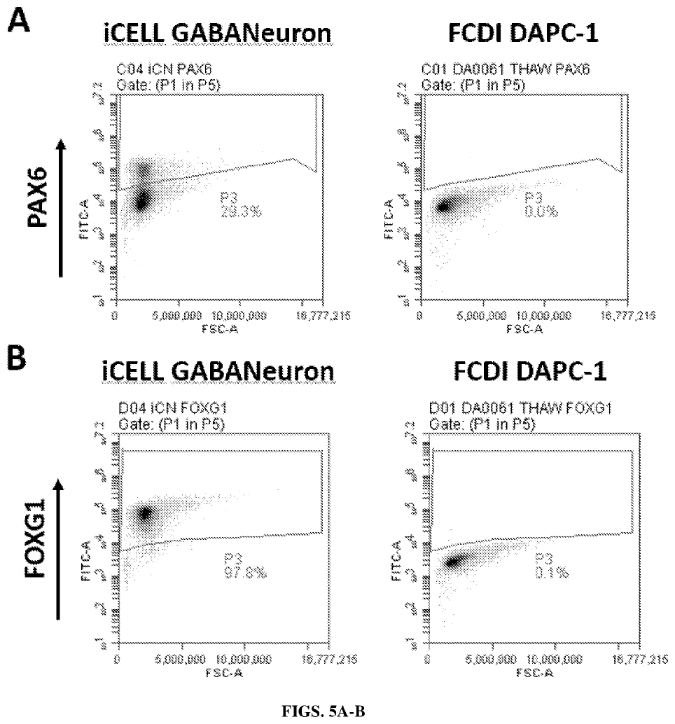

MAP2/Nestin flow cytometry assay was used to identify the percentage of cells with the potential to become mature (post-mitotic) neurons by 14 days post-thaw. Results from a representative batch are shown in FIG. 4. The mutually exclusive Nestin co-stain was included for better separation and gating of the MAP2+ population.

TABLE 6

| | | | |
|---|---|---|---|
| | | | LifeTech Alexa |
| Marker | Vendor, Catalog | Dilution | Fluor ® (1:1000) |

Flow Cytometry

| | | | |
|---|---|---|---|
| Rabbit-FOXA2 | Cell Signaling, 8186 | 1:500 | A21244 |
| Mouse-FOXA2 | Abcam, ab60721 | 1:10,000 | A31571 |
| Rabbit-LMX1 | Millipore, AB10533 | 1:10,000 | A11008 |
| Mouse-NURR1 | ThermoFisher, MA1-195 | 1:1,000 | A21202 |
| Mouse-MAP2 + Alexa488 | Millipore, MAB3418X | 1:1,000 | NA |
| Mouse-Nestin + Alexa647 | BD, 560393 | 1:20 | NA |
| Mouse-TH | Sigma, T2928 | 1:8500 | A21121 |

Immunocytochemistry

| Mouse-FOXA2 | Abcam, ab60721 | 1:10,000 | A21131 |
|---|---|---|---|
| Rabbit-LMX1 | Millipore, AB10533 | 1:5,000 | A21244 |
| Mouse-NURR1 | ThermoFisher, MA1-195 | 1:1,000 | A21131 |
| Mouse-TH | Sigma, T2928 | 1:10,000 | A21202 |
| Mouse-MAP2 + Alexa488 | Milipore, MAB3418X | 1:1,000 | NA |
| Mouse-Nestin + Alexa647 | BD, 560393 | 1:20 | NA |
| Rabbit-BARHL1 | Novus Biologicals, NBP1-86513 | 1:1,000 | A31573 |
| Sheep-PITX2 | R&D Systems, AF7388 | 1:1,000 | A11015 |
| Immunohistochemistry | | | DAB or IFC |
| Mouse-hNuclei | Millipore, MAB1281 | 1:1,000 | DAB |
| Mouse-hNuclei | Millipore, MAB1281 | 1:500 | IFC |
| Rabbit-TH | Pelfreez, P40141 | 1:1,000 | DAB |
| Rabbit-TH | Pelfreez, P40141 | 1:500 | IFC |
| Mouse-hKi67 | Cell Signaling, 9027S | 1:1,000 | DAB |
| Rabbit-5-HT | Millipore, S5545 | 1:2,000 | DAB |
| Rabbit-Iba1 | Wako, 019-19741 | 1:2,000 | DAB |
| Rabbit-GFAP | SC123, Y40420 | 1:2,000 | DAB |
| Goat-FOXA2 (HNF-3B) | R&D Systems, AF2400 | 1:200 | IFC |
| Mouse-FOXA2 | Abcam, ab60721 | 1:200 | IFC |
| Rabbit-GIRK2 (Kir3.2) | Alamone Labs, APC-006 | 1:500 | IFC |
| Sheep-Calbindin | Cell Signaling, 2173 | 1:300 | IFC |
| Horse anti-Mouse | Vector Labs, BA-2001 | 1:200 | NA |
| Goat anti-Rabbit | Vector Labs, BA-1000 | 1:200 | NA |
| Donkey anti-Sheep AF-488 | Invitrogen, A-11015 | 1:200 | NA |
| Donkey anti-Rabbit AF-488 | Invitrogen, A-21206 | 1:200 | NA |
| Donkey anti-Rabbit AF-555 | Invitrogen, A-31572 | 1:200 | NA |
| Donkey anti-Mouse AF-647 | Invitrogen, A-31571 | 1:200 | NA |
| anti-Mouse AF-488 | Invitrogen, A-21202 | 1:200 | NA |
| anti-Goat-AF-488 | Invitrogen, A-11055 | 1:200 | NA |

TABLE 7
Significance of qPCR (FIG. 1).
One-way ANOVA with Bonferroni post-hoc test.
(* P < 0.05, ** P < 0.01, *** P < 0.001)

| | D17 vs. D24 | D17 vs. D37 | D24 vs. D37 |
|---|---|---|---|
| otx2 | * | *** | *** |
| foxa2 | *** | *** | * |
| lmx1a | * | ns | * |
| en1 | ** | ** | ns |
| pitx3 | *** | ns | *** |
| nurr1 | ns | ns | ns |
| TH | ns | ns | ns |
| Dat | ns | ns | ns |
| Girk2 | ns | ns | ns |
| Calb | ns | ns | ns |
| etv5 | *** | *** | ns |
| cnpy1 | ns | ns | ns |
| spry1 | ns | ns | ns |
| Dbx1 | ns | ns | ns |
| pitx2 | ns | ns | ns |
| barhl | ns | ns | ns |
| hoxa2 | ns | ns | ns |
| Lhx2 | ns | ns | * |
| Foxg1 | * | * | ns |
| Pax6 | ns | ns | ns |
| BRN3a | ns | ns | ns |
| Phox2a | * | ** | ns |
| HB9 | * | * | ns |
| Chat | ns | ** | ns |
| Vglut1 | ns | ns | ns |
| Gad1 | * | ns | * |
| Sert | *** | ns | *** |
| Glast | * | ** | ns |
| S100b | ns | ns | ns |
| CD44 | ns | ns | ns |
| Sox1 | ns | ns | ns |
| Dcx | ns | ** | ns |
| Neun | ns | ** | * |
| Nkx2.1 | ns | ns | ns |
| Eomes | ns | ns | ns |

Dopamine secretion by cells similar to FCDI DAPC-1 (“PD Therapy Cells”, a process variation from earlier in development) was measured after culturing for 5 weeks in Maturation Medium. The concentration of dopamine released during a 30 min incubation in HBSS was measured. Higher values were obtained after cell depolarization (HBSS+56 mM KCl).

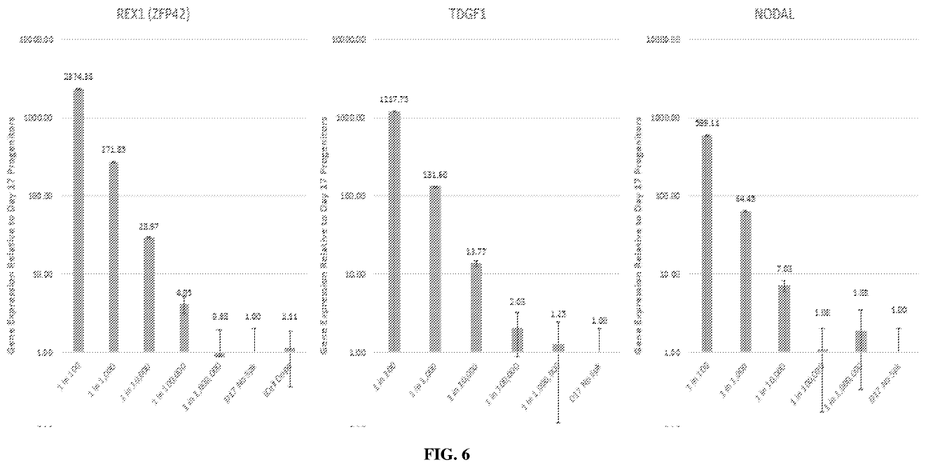

FCDI DAPC-1 cells were stained with anti-PAX6 (Biolegend #901301) (FIG. 5A) or anti-FOXG1 (FIG. 5B). iCell GABA Neurons (FCDI) are shown as a positive control; they are cells patterned to a forebrain phenotype, predominantly GABAergic, and contain a subpopulation of PAX6+ neurons and also FOXG1+ neurons. Results are shown in FIGS. 5A-B.

RT-QPCR assays for REX1, TDGF1 and NODAL can detect inhibitory post-synaptic currents (iPSCs) spiked into DA progenitor cells (FCDI DAPC-1 process). The REX1 assay is the most sensitive, reproducibly detecting one iPSC in 100,000 FCDI DAPC-1 process cells. Results are shown in FIG. 6.

TABLE 3
Detection of iPSCs spiked into FCDI DAPC-1
process (5%) on process day 5.

| | | Control | 5% Spike |
|---|---|---|---|
| Day 5 | Oct4 | 1.2% | 6.7% |
| | Tra-1-81 | 99.9% | 99.9% |
| Day 11 | Oct4 | nd | 0.7% |
| | Tra-1-81 | nd | 9% |
| Day 17 | Oct4 | 0.3% | 0.1% |
| | Tra-1-81 | 0.1% | 0.0% |

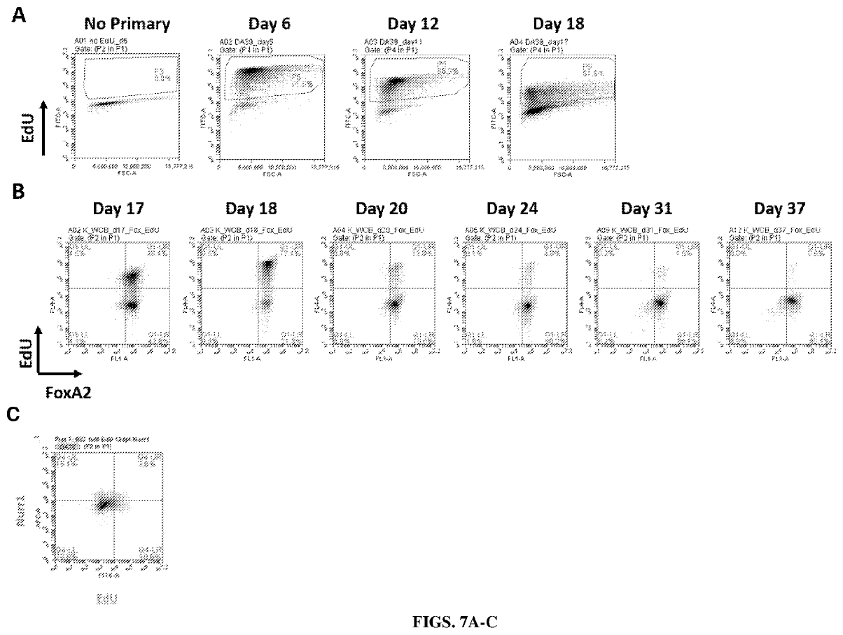

As shown above, the dopaminergic neuron progenitor cells displayed phenotypic markers (FIG. 1 and FIG. 2) and developmental potential similar to dopamine neurons precursors (FIG. 3, FIG. 4) found in the substantia nigra region of the developing midbrain. FCDI DAPC-1 lacks significant forebrain neurons and residual iPSCs that could be detrimental to therapeutic use (FIGS. 5A-B, FIG. 6, and Table 3). Importantly, and unlike other DA cell therapy products, FCDI DAPC-1 was observed to be a proliferating progenitor cell population as demonstrated by EdU incorporation (FIG. 7).

### Example 10

### Reduction of Motor Deficits in a PD Animal Model In Vivo

An animal model of Parkinson's Disease (PD), 6-OHDA lesioned athymic nude rats (RNU, Crl:NIH-Foxn1^{rnu}), was used for further studies. These animals display significant motor defects, which can be observed using the amphetamine induced rotation test (Blesa et al., 2014; Campos et al., 2013; Deumens et al., 2002; Vermilyea, et al., 2018). Dopaminergic progenitor neurons (D19) produced as described in the Examples above were administered to the substantia nigra of mice to determine if this would alleviate motor defects in the animals as observed using the amphetamine induced rotation test. As discussed below, while mature D37 neurons did not improve motor defects in animals, administration of the D17 and D19 dopaminergic progenitor neurons to the mice were able to completely reverse these motor defects by 6 months in vivo.

Rats with unilateral damage to the nigrostriatal dopamine system (e.g., induced by neurotoxins, such as 6-hydroxydopamine, overexpression of α-synuclein, or injections of toxic synuclein protofibrils) have been used as experimental models to mimic the loss of dopamine neurons seen in Parkinson's disease. The amphetamine rotation test is commonly used to monitor the extent of motor impairment induced by the lesion, and this test has also become the standard tool to demonstrate transplant-induced functional recovery or the efficacy of neuroprotective interventions aimed to preserve or restore DA neuron function. This test is described, e.g., in Wakeman et al., 2017.

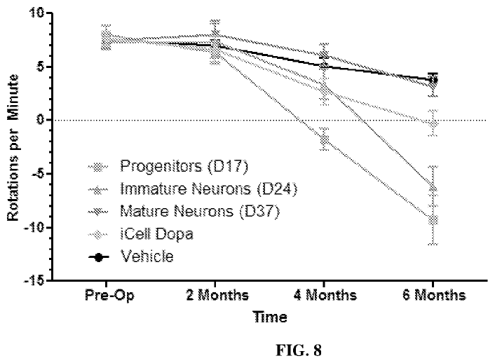

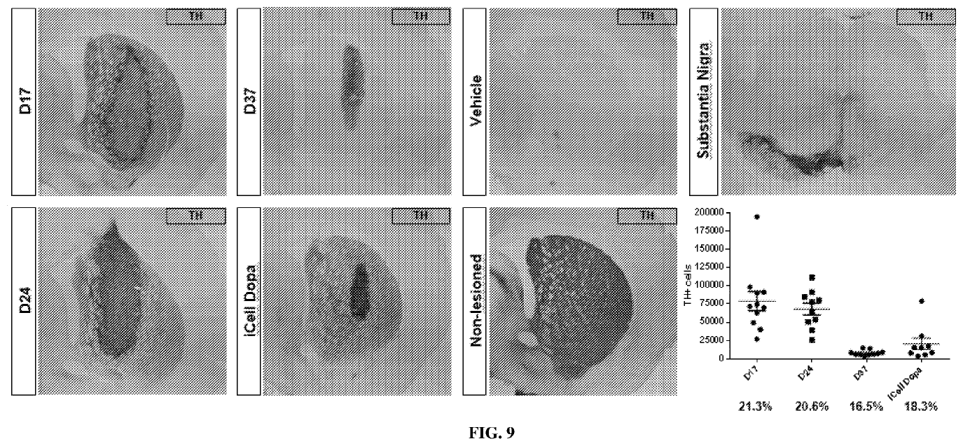

Amphetamine rotations were tested in the rat PD model as described above. As shown in FIG. 8, administration of day 17 (D17) dopaminergic neuronal precursor cells resulted in alleviation of motor symptoms in the rats by 6 months, as observed with the amphetamine rotations test. D24 immature neurons improved motor performance, although the effect from the D24 neurons appeared to be less than the effect of the D17 neurons, which was particularly notable at the 4-month and 6-month timepoints.

Immunohistochemistry staining of brain slices was performed in brain slices at 6-months after administration of the neurons to the striatum of rats. Increased NCAM expression was observed after administration of D17 or D24 neurons, as compared to mature D37 or iCell® Dopa neurons. These results indicate that progenitor (D17) and immature (D24) mDA neurons outperform more mature (D37) mDA neurons in engraftment.

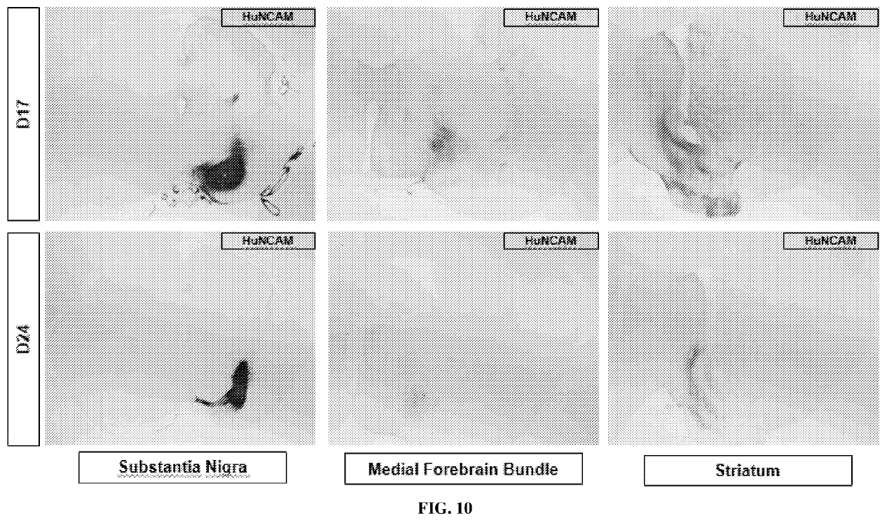

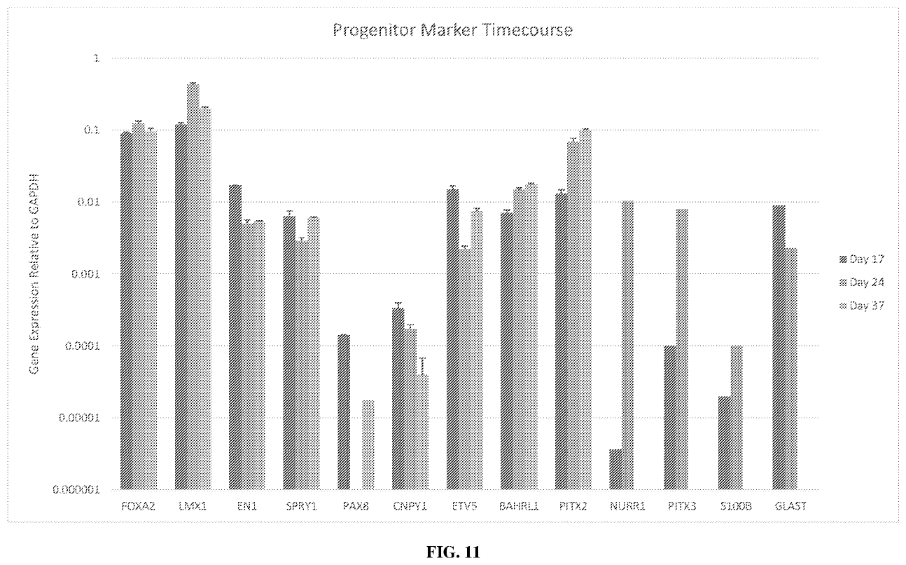

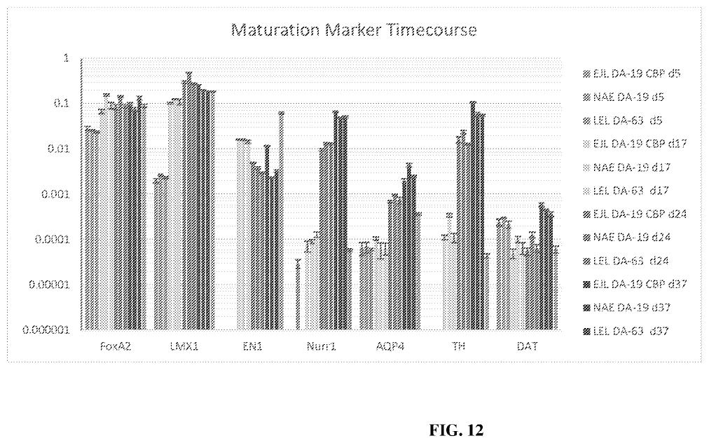

Striatal re-innervation was observed at 6 months post-transplant. Innervation of the striatum by the D17 cells appeared to be the highest, as compared to the other neurons tested. D17 and D24 cells displayed marked improvements in innervation as compared to D37 or iCell® Dopa neurons. Results are shown in FIG. 9, and examples of intranigral innervation of grafts into the striatum are shown in FIG. 10.

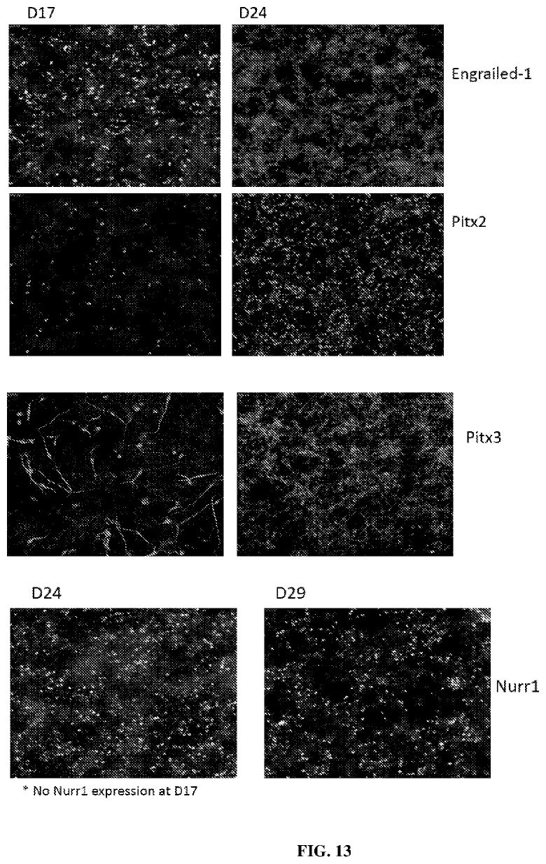

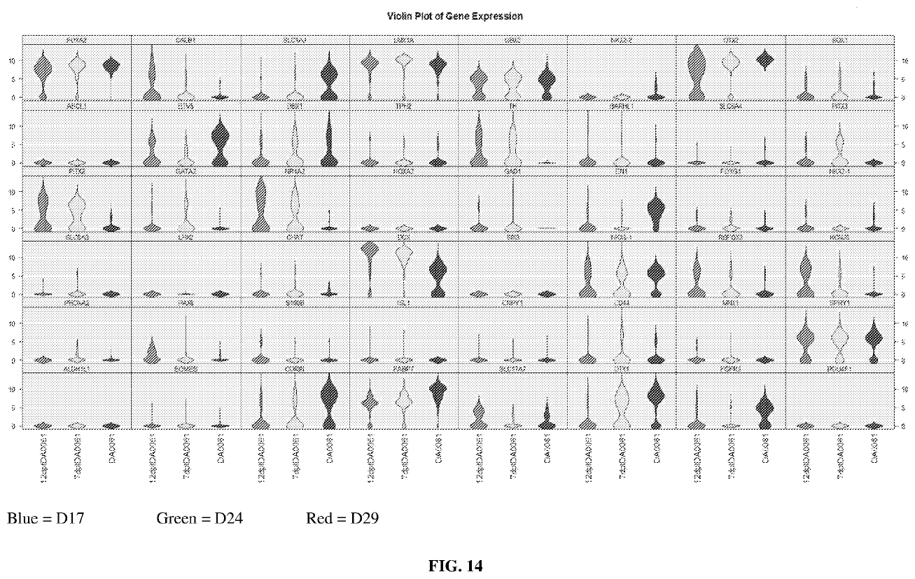

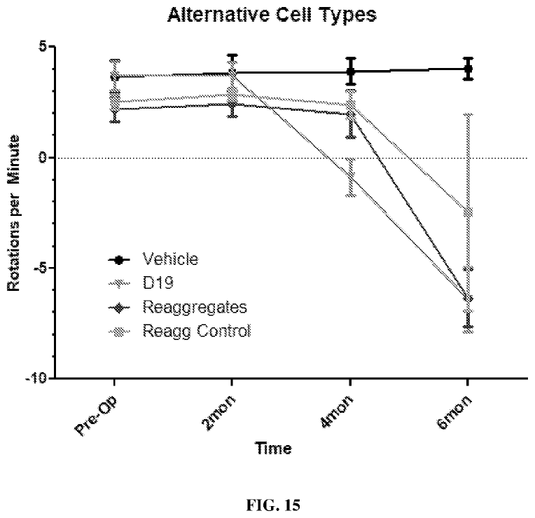

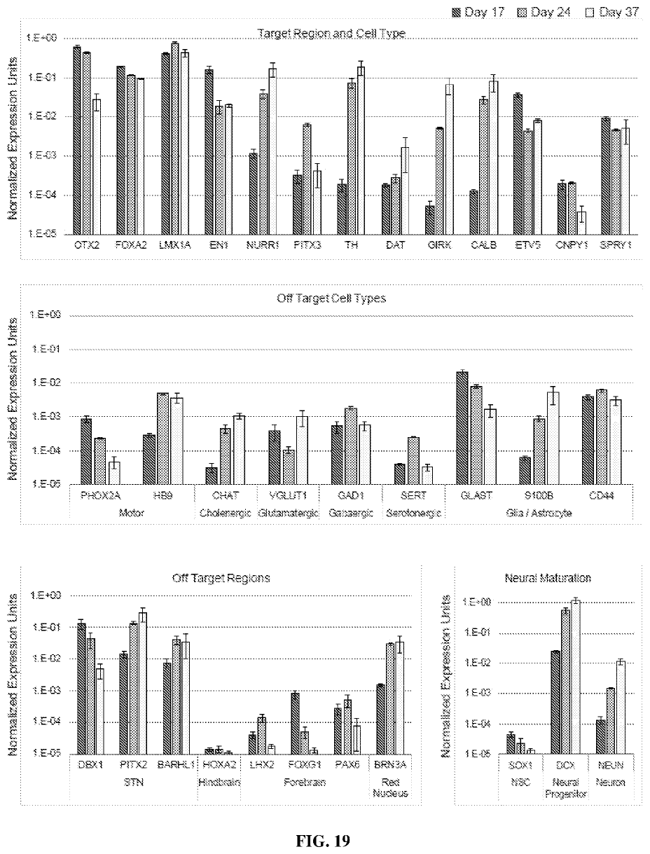

Progenitor markers were measured in the D17, D24, and D37 cells using qPCR. When comparing the D17 and D24 cells, Lmx1, Nurr1, and Pitx3 are expressed at a higher level in D24 cells whereas En-1, Pax8, ETV5, and Glast are expressed at higher levels in the D17 cells (FIG. 11). Maturation markers were also measured across the cells, and AQP4 and tyrosine hydroxylase (TH) are expressed at higher levels in D24 compared to D17 cells (FIG. 12). Additional data regarding normalized expression of different genes in different cell types generated after varying durations of differentiation (at D17, D24, and D37 timepoints) are shown in FIG. 19. These results are consistent with increased differentiation of the D24 cells into mature dopaminergic neurons, as compared to the D17 dopaminergic neuronal precursor cells. Immunocytochemistry was also performed on the D24 and D17 cells, and results are shown in FIG. 13. Results from the immunocytochemistry (ICC) experiments were consistent with the qPCR findings.

Functional testing of alternative cell types showed that administration of D19 “intermediate” dopaminergic cells was able to completely reverse motor deficits by the 6-month timepoint. These cells followed the method described in Table 2, until D15 in which they were plated on LN521 in D17 plating medium, and then fed Neuron Maturation Medium Minus DAPT D16-18, and frozen on D19; with the modification that CHIR concentration was changed from 1.75 to 1.65 μM and with benzonase added to the D5 and D17 quench media. The D19 animals started showing functional improvements by 4 months and this group saw a more rapid improvement compared to the Reaggregates (D17 cells dissociated and reaggregated to a smaller size overnight and frozen on D18) or their control cells (The D17 cells from which reaggregates were made). The Reaggregates and their control cells maintained motor deficits through the 4-month time point before improvements were realized. The total number of cells injected for each animal was; D19 on average 290 k per animal, Reaggregates on average 333 k, and Reaggregate Control on average 369 k per animal. Multiple animals in each of the Reaggregate and Reaggregate Control group (N=3) were tested. The D19 animal group had N=10 animals. Results are shown in FIG. 15.

### Example 11

### Expression of D17 Dopaminergic Precursor Neurons

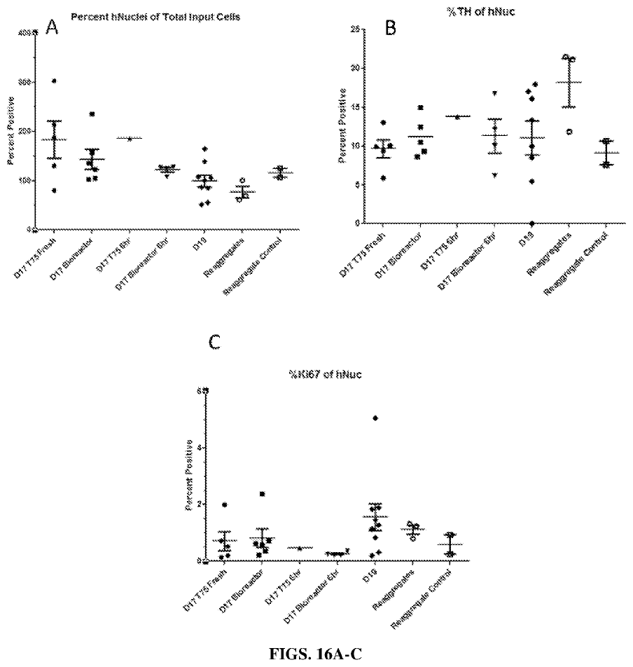

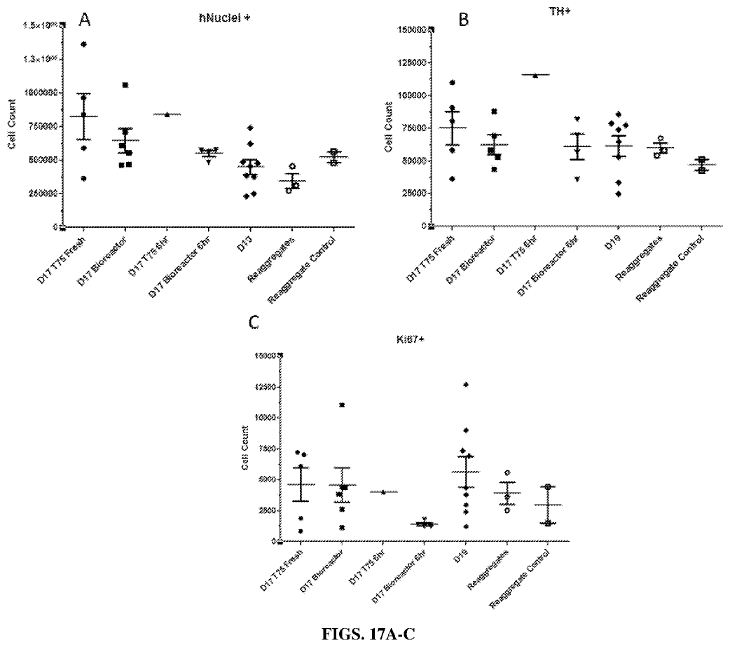

Brain slices were stained 6-month post engraftment for the presence of human nuclie (hNuc), tyrosine hydroxylase (TH) and Ki67. TH is involved in the production of dopamine by dopaminergic neurons and dopaminergic neuronal precursor neurons. Ki67 is a gene involved with cell proliferation. h-Nuc is a gene marker expressed by the neuronal precursor cells and was measured to evaluate if further cell expansion occurred after engraftment. Results are shown in FIG. 16. A full series of 40 μm coronal sections stained for HuNuclei using the DAB method were counted at 60× magnification using Stereo Investigator optical fractionator (Microbrightfield Bioscience, Version 10.40). TH (every 12^{th} serial section) and HuNuclei (every 12^{th} serial section) stereological parameters were frame size (75 μm×75 μm) and grid size 250 μm×250 μm) to count 9% of the total graft area with average CE=0.13 for HuNuclei (Gundersen m=1); frame size (80 μm×80 μm) and grid size 225 μm×225 μm) to count 12.6% of the total graft area with average CE=0.17 for TH (Gundersen m=1). Percentages were calculated based on the calculated numbers of hNuc, TH, and Ki67 positive cells in each graft. The total calculated number of cells divided by the total input number of cells results in percent positive.

As shown in FIGS. 16A-C, each group mean shows more than 100% positive for hNuc, indicating cell expansion after engraftment. At the 6-month time point of sacrifice the Ki67 positive population accounts for less than 1% of the hNuc population on average with the exception of D18 and Reaggregates. This low percentage of Ki67 supports the idea that the cells are no longer proliferating after 6 months engrafted but does not reflect the proliferative ability of the engrafted cells early after the engraftment date. Having an average hNuc positive greater than 100% for all groups suggests a proliferative cell type early after engraftment that changed into a definitive cell type that no longer proliferates but retains its human origin marker. The percentage of TH positive cells is much lower in this animal study than previously seen. Averages for these groups are around 10-15% whereas previously the inventors have seen average percent TH+ in the range of 20-30%.

Stereology Analysis for hNuc, TH, and Ki67 was performed. Every 12th section (½ series) was stained for hNuclei, TH, or hKi67 and quantified by unbiased stereology. For each animal, the graft area was outlined and counted. Each graph has a unique Y-axis. Results are shown in FIGS. 17A-C.

The number of hNuc positive cells from each animal in each test group, including the mean and standard error of the mean (SEM), are shown in FIG. 17A. The use of this marker demonstrates the cell that is hNuclei-ir is of human origin (injected test material). The D17 T75 fresh group shows the largest range of engrafted hNuc+ cells compared to all other groups. All other groups appear to have consistent engraftment of cells between all animals in that group. The mean for each group varies across samples. Analysis using a 1 way ANOVA test indicates there is a statistical significance in the mean number of hNuclei+ cells in the D17 T75. Fresh and D19 groups (p=0.0384).

The number of TH positive cells from each animal in each group, including the mean and SEM, are shown in FIG. 17B. TH-ir positive cells indicate a cell type able to produce dopamine and that the cell is from the test material due to the ablation performed prior to transplant. Apart from the D17 T75 6 hr group (which only had stains from one animal to quantitate) all the groups show similar numbers of TH+ cells engrafted with a mean at roughly 60,000 cells. One-way ANOVA testing indicates there is no statistical difference between these treatment groups for TH engraftment.

FIG. 17C shows the number of Ki67 positive cells from each animal in each group, the mean and SEM. Ki67-ir cells indicate a cell type that is capable of division/propagation. The specific antibody used in this assay is human specific and will only bind to cells of human origin. These results indicate that administered cells display very low rates of cell proliferation.

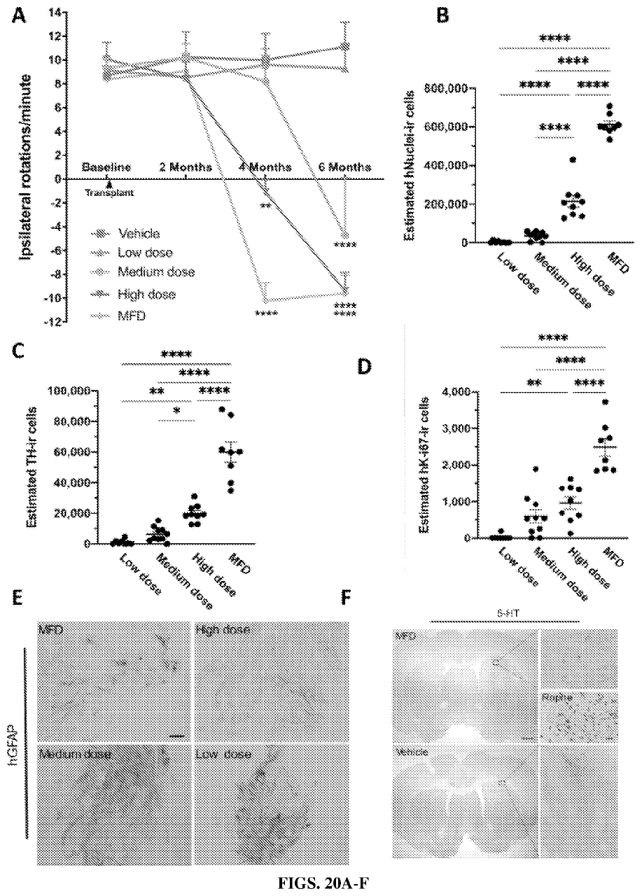

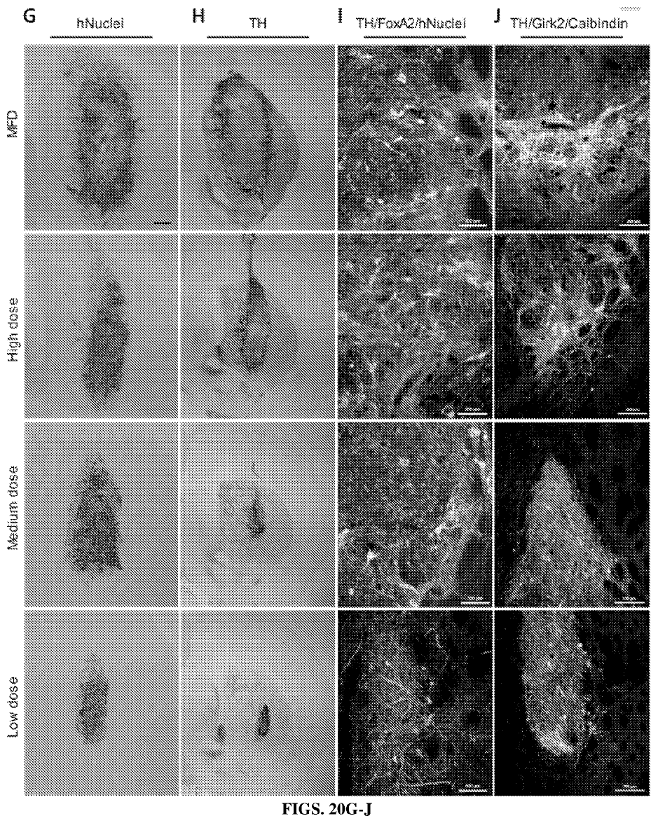

Improvements in behaviors in vivo were observed in 6-OHDA lesioned animals that were administered the D17 cells. Characterization and analysis of function, survival, and innervation of D17 progenitors in vivo are shown in FIGS. 20A-J. Time-based analysis of d-amphetamine-induced rotations measured pre-operatively and at 2, 4, and 6 months post-engraftment (FIG. 20A). Stereological estimates of hNuclei-ir cells contained in grafts of low, medium, high, or maximum feasible dose (FIG. 20B). Quantification of stereological estimates of TH-ir cells (FIG. 20C) and stereological estimates for each group (FIG. 20D) were performed. Graft sections showed positive staining for hGFAP (FIG. 20E), 5-HT (FIG. 20F). Low, medium, high, and maximum feasible dose D17 cells were imaged for hNuclei (FIG. 20G), TH (FIG. 20H), immunofluorescent triple-labeled hNuclei/TH/FoxA2 (FIG. 20I), and TH/Girk2/Calbindin (FIG. 20J). These results demonstrate that the D17 cells can be administered to restore behavioral capabilities in vivo as observed using an animal model of Parkinson's disease.

### Example 12

### Materials and Methods

The following methods were used in experiments described in Examples 10-12.

Lesioning and Engraftment: Female nude rats received 6-OHDA lesioning at 8-9 weeks of age. The neurotoxin was administered directly to the medial forebrain bundle while the rats were anesthetized in a stereotactic apparatus. Rats were tested every three weeks post lesioning using amphetamine to score rotations measured using a Rotometer. Animals indicating successful lesioning (rotations ≥ 5/min over a 30 min period) were randomly distributed into experimental treatment groups based on amphetamine rotation data to receive cells or a vehicle control. Freshly prepped cells were injected at a concentration of 150,000 cells/μL in a volume of 3 μL (450,000 cells per animal) directly into the striatum of the rat.

Rotation Measurements: After lesioning, animals showed rotational behavior (circling) towards the lesioned side, indicating lesion success. This behavior was induced using amphetamine which increases the amount of dopamine in the brain. After allowing the rat to acclimate to the chamber for 5 minutes, rotations were tracked for 90-minutes, binned every 5-minutes, and average net rotations-per-minute were calculated. Amphetamine rotations were measured every 2 months post-engraftment (FIG. 1). Apomorphine injections were used to track rotations in the opposite direction of the lesioned hemisphere. Apomorphine induced rotations were tracked for 60-minutes and measured every 3 months post-engraftment (FIG. 2).

Post-mortem Analysis: Rats (6 months) were anesthetized and perfused transcardially with ice-cold 0.9% saline followed by 4% paraformaldehyde. Brains were removed and post-fixed in 4% paraformaldehyde for 18-24 hours before being placed in a sucrose gradient (10%, 20%, 30%) and allowed to sink. All brains were sectioned into 40 μm coronal sections on a frozen sledge microtome and processed for immunohistochemistry using 3,3′-Diaminobenzidine (DAB) with nickel enhancement where applicable or fluorescence immunohistochemistry. Stereological parameters for TH and HuNuclei (½ series) were frame size (80 μm×80 μm) and grid size (350 μm×350 μm) to count 9% of the total area with average CE=0.14 for TH (Gundersen m=1) counted at 60× magnification. Sections (every 12th section) containing graft were stained for Ki-67. The entire area of the graft body (100%) was counted using an Olympus BX61. The number of Ki67+ cells is calculated as 12×the sum of the number of Ki-67+ cells in the 5 sections counted.

### Example 13

### Characterization of mDA Precursor Cells In Vitro

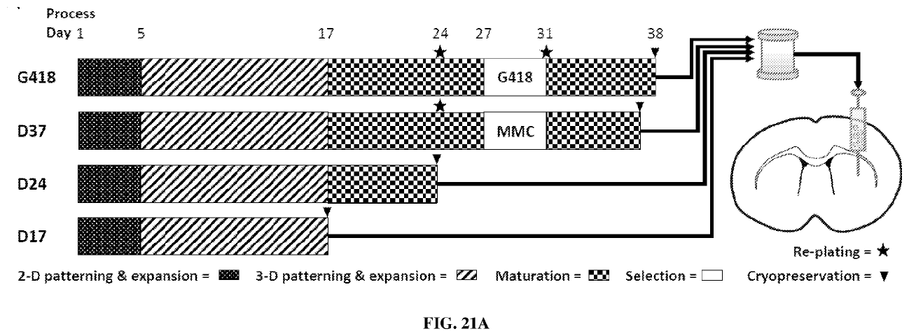

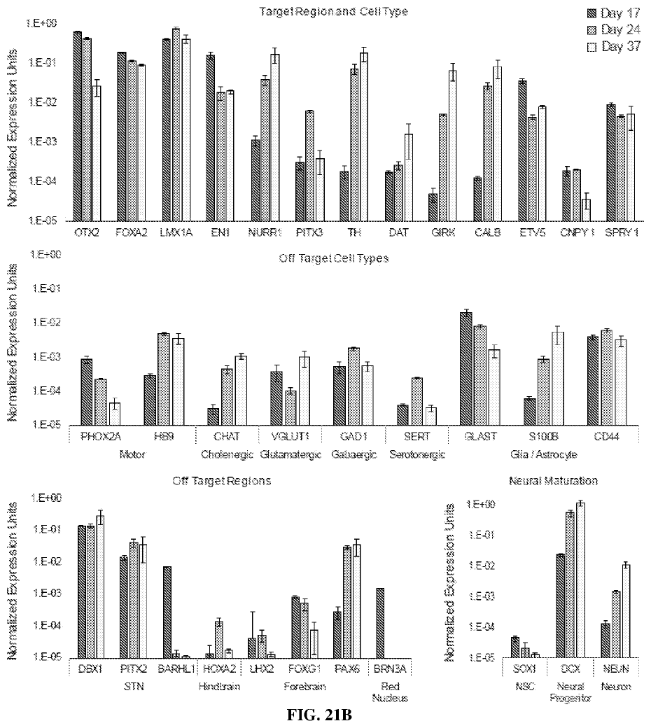

Previous transplantation studies utilized research-grade iPSC-derived mDA neurons, and cells made using variations of the same differentiation protocol (Hiller et al., 2020; Wakeman et al., 2017). For the next stages in the development of a cell therapy, additional steps were taken to transition to a process suitable for cGMP manufacturing and clinical use. A clinical grade human iPSC line was used. An iPSC master cell bank and working cell bank were manufactured under cGMP conditions. The early stage of iPSC-mDA differentiation was adapted by altering the timing and concentration of small molecule inhibitors. To address safety and regulatory concerns, the raw materials used in the differentiation process were clinical grade where possible. The iPSC-mDA neurons differentiated to the most advanced maturational stage (D37) were enriched during the differentiation process using a low concentration of mitomycin C to remove proliferative cells as previously described (Hiller et al., 2020) (FIG. 21A). This approach bypassed the need for the drug selection cassette used in the R&D grade G418 cells. The mDA progenitor (D17) and immature (D24) mDA neurons cannot be enriched with mitomycin C because they are still proliferative; thus a major goal of these experiments was to determine whether the adapted differentiation process (without an enrichment step) was adequate to prevent unwanted cell proliferation in grafted D17 and D24 cells.

Previous studies provided evidence that the human iPSC-mDA neurons can express high levels of regional midbrain markers and low levels of forebrain and hindbrain markers (Hiller et al., 2020; Wakeman et al., 2017). A similar gene expression panel was used to characterize cells made using the differentiation process adapted for translational use (FIG. 21B, Table 5). All differentiation stages (Days 17, 24, and 37) expressed regional midbrain markers OTX2, FOXA2, and LMX1A at high levels. EN1 was most highly expressed at D17, decreased by D24, and maintained that level of expression through D37. More mature mDA markers (NURR1, TH, DAP, GIRK, CALB) were either expressed at very low levels or not at all on D17 and showed a progressive increase from D24 to D37. PITX3 expression was highest at D24. Markers reported to be predictive of good engraftment (Kirkeby et al., 2017), ETV5 and SPRY1, were expressed at all stages, while CNPY1 had low expression at D17 and D24 and was nearly undetectable by D37. Expression levels of markers for non-mDA cell types such as motor (PHOX2A, HB9), cholinergic (CHAT), glutamatergic (VGLUT1), GABAergic (GAD1), and serotonergic (SERT) neurons were low/not expressed across all differentiation stages. The most highly expressed off-target marker was GLAST, indicating that some astrocyte precursors were present in the culture. Consistent with the presence of STN neurons, which express some of the same molecular markers of mDA neurons (Kee et al., 2017; Nouri & Awatramani, 2017), expression of DBX1, PITX2, and BARHL1 was observed at all stages of differentiation. The hindbrain marker HOXA2 was not expressed, and low levels of forebrain markers were detected throughout D17-37. Flow cytometry demonstrated that <1% of D17 cells express FOXG1 or PAX6, indicating a lack of forebrain neuron progenitors. BRN3A which is expressed in the red nucleus in the midbrain (Agarwala, Sanders, & Ragsdale, 2001; Wallen et al., 1999) was also detected. At all time points tested (D17, D24, and D37) the marker of neural stem cells SOX1 was not expressed, indicating that the cultured cells had passed the stem cell stages of differentiation. At each of the three time points the neural progenitor marker DCX was expressed, while expression of the more mature neural marker NEUN increased from D17 to D37.

TABLE 5

| Gene | TaqMan Assay |
|---|---|
| OTX2 | Hs00222238_m1 |
| FOXA2 | Hs00232764_m1 |
| LMX1A | Hs00898455_m1 |
| EN1 | Hs00154977_m1 |
| NURR1/NR4A2 | Hs00428691_m1 |
| PITX3 | Hs01013935_m1 |
| TH | Hs00165941_m1 |
| SLC6A3/DAT | Hs00997374_m1 |
| GIRK2/KCNJ6 | Hs01040524_m1 |
| CALB | Hs00191821_m1 |
| ETV5 | Hs00927557_m1 |
| CNPY1 | Hs01073160_m1 |
| SPRY1 | Hs01083036_s1 |
| DBX1 | Hs01380082_m1 |
| PHOX2A | Hs00605931_m1 |
| HB9/MNX1 | Hs00907365_m1 |
| CHAT | Hs00758143_m1 |
| VGLUT1 | Hs00220404_m1 |
| GAD1/GAD67 | Hs01065893_m1 |
| SLC6A4/SERT | Hs00169010_m1 |
| GLAST/SLC1A3 | Hs00188193_m1 |
| S100B | Hs00902901_m1 |
| CD44 | Hs01075864_m1 |
| PITX2 | Hs04234069_mH |
| BARHL1 | Hs01063929_m1 |
| HOXA2 | Hs00534579_m1 |
| LHX2 | Hs00180351_m1 |
| FOXG1 | Hs01850784_m1 |
| PAX6 | Hs00242217_m1 |
| POU4F1/BRN3A | Hs00366711_m1 |
| SOX1 | Hs01057642_s1 |
| DCX | Hs01035496_m1 |
| NeuN/RBFOX3 | Hs01370653_m1 |
| GAPDH | 4333764F |

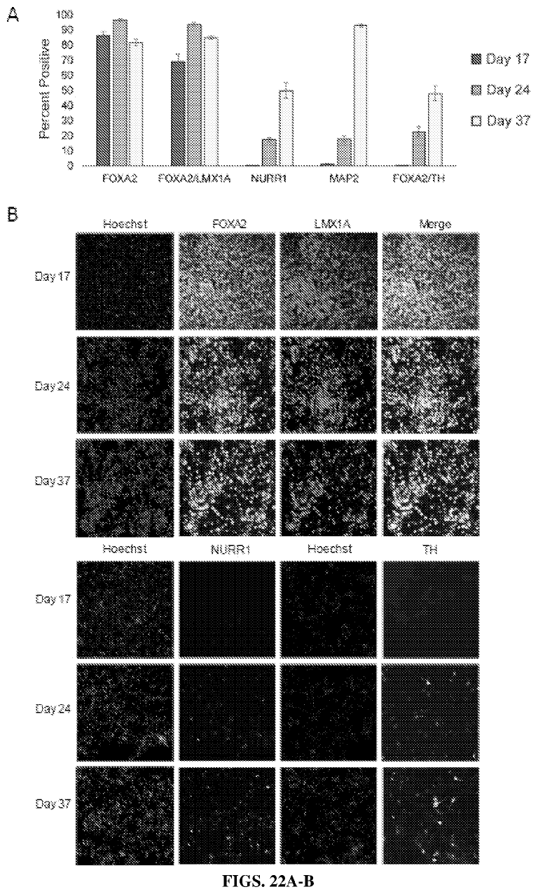

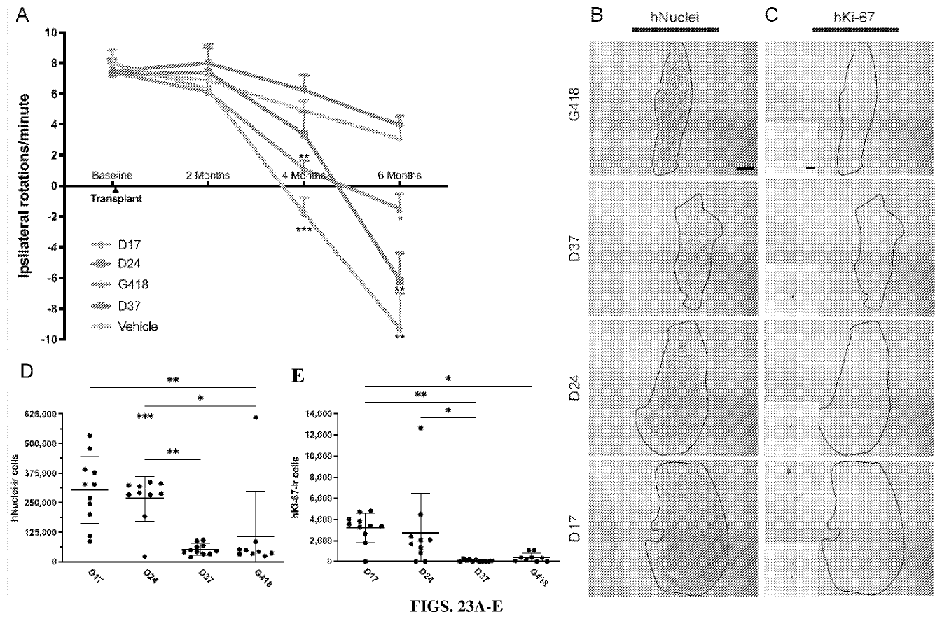

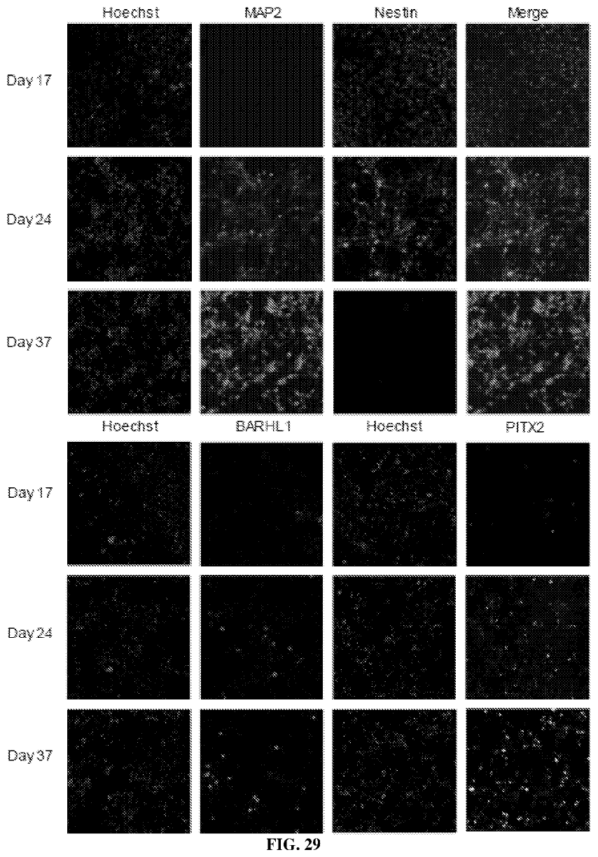

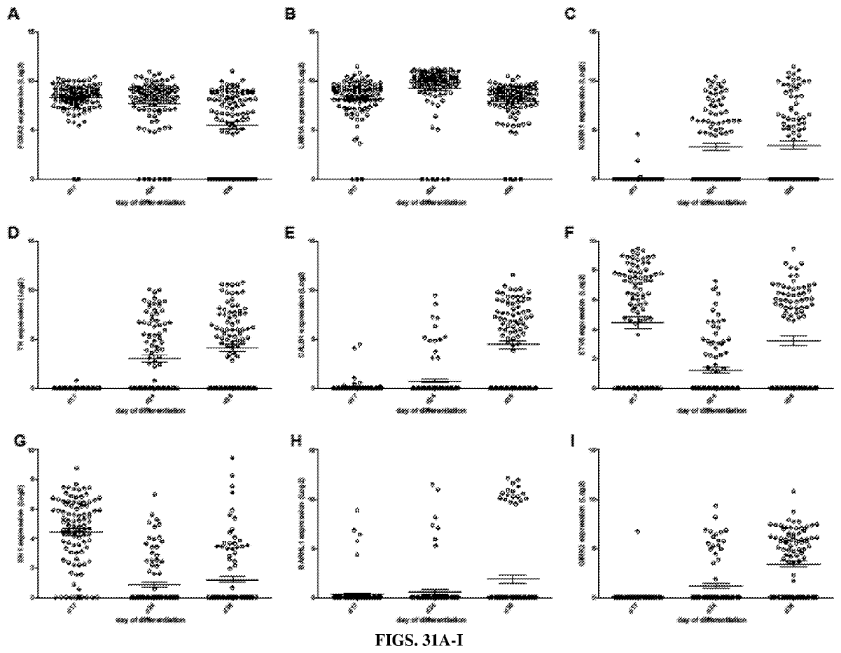

Flow cytometry was then used to examine the mDA population at the protein level (FIG. 22A) and single cell PCR to examine the mDA population at the RNA level (FIGS. 31A-I). The percentage of FOXA2-immunoreactive (ir) cells remained high (>80%) from D17 through D37, while co-expression of FOXA2 and LMX1 was around 70% at D17, increasing above 90% by D24. This population of FOXA2/LMX1-ir cells remained high (˜85%) in D37 cultures. As expected and consistent with the qPCR results, more mature markers such as NURR1, MAP2, and TH were not detected in D17 samples. The total population percentages of each of these three markers increased over time with approximately 20% being immunoreactive for each in D24 samples, and 50% (NURR1, FOXA2/TH) or 90% (MAP2) being immunoreactive in D37 samples. Immunocytochemistry was used to visually identify these populations of cells (FIG. 22B, FIG. 29). Consistent with the flow cytometry results, LMX1A and FOXA2 were co-expressed in a high percentage of cells at each developmental timepoint. Also consistent with the flow cytometry, NURR1− and TH-ir cells were not present at D17, while a smattering was seen by D24, and a higher number of cells, as well as brighter individual cells, were observed at D37. MAP2 was not detected in D17 samples but became increasingly expressed over time with robust MAP2-ir at D37. Inversely, Nestin-ir cells were abundant on both D17 and D24, but nearly undetectable at D37. STN markers BARHL1 and PITX2 were detected at all time points with few immunoreactive cells present at D17 and an increasing number of cells detected over time. A small percentage of the D37 cells express BARHL1, suggesting that STN neurons are a minority subset of the NURR1-ir cells, and are significantly outnumbered by immature mDA neurons.

Together, these data show that the differentiation protocol resulted in production of cultures with a primarily midbrain phenotype that include cells from regions close to the SN, including the STN and red nucleus cells. Furthermore, minimal contamination of forebrain or hindbrain cells was observed. The D17 cells were observed to be at a progenitor stage and did not express NURR1 or other markers characteristic of mature mDA neurons, other than EN1.

### Example 14

### Effect of Cellular Maturity on Transplant Survival and Function

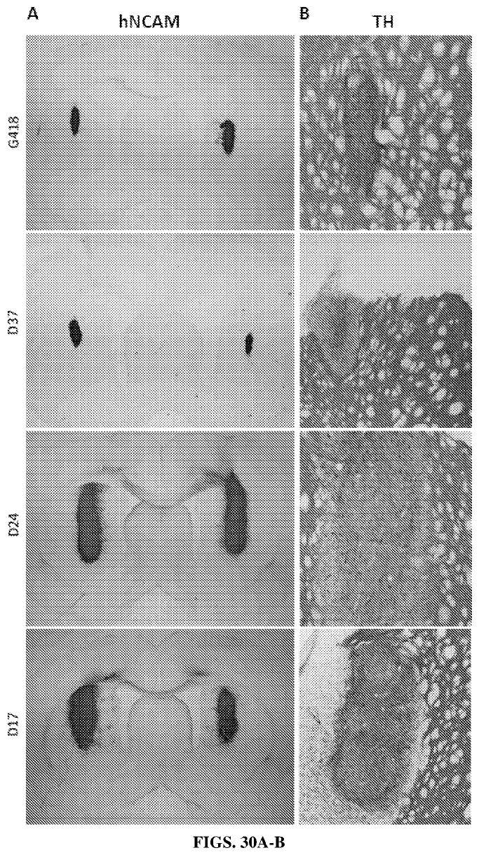

To evaluate the effect of cellular maturity on transplant survival, grafts of D17, D24, D37, or G418 cells were injected into the striatum bilaterally of intact athymic rats. At 3 months post-transplantation (FIGS. 30A-B), coronal sections stained for human-specific neural cell adhesion molecule (hNCAM) revealed relatively small G418 and D37 grafts, with few hNCAM-ir fibers innervating the host striatum. In contrast, large hNCAM-ir grafts, and their processes, were visible in animals engrafted with either D17 or D24 cells. While all grafts contained TH-ir neurons, only D17 grafts were cytoarchitecturally arranged in a manner similar to what is characteristically seen with fVM implants, with dopaminergic cell bodies localized at the periphery of the grafts (L. Thompson, Barraud, Andersson, Kirik, & Bjorklund, 2005).

After observing that the modified differentiation protocol produced cells that are viable in the immunocompromised intact rat brain, we performed a long-term functional study. Rats with unilateral 6-OHDA-induced medial forebrain bundle (MFB) lesions confirmed by repeated d-amphetamine-induced rotations were transplanted with vehicle control or D17, D24, D37, or G418 cells (150,000 cells/μL; 3 μL; n=9-11/group) and sacrificed 6 months post-injection. A summary table (Table 4) describes the histological and behavioral findings for each cell type and dosing group.

TABLE 4

| | | | | | D17, | D17, | D17, | |
|---|---|---|---|---|---|---|---|---|
| Endpoint\ | | | | | Low | Medium | High | D17, |
| Treatment | G418 | D37 | D24 | D17 | Dose | Dose | Dose | MFD |
| Behavioral | Yes, | No | Yes, | Yes, | No | Yes, | Yes, | Yes, |
| recovery | by 6 MPI | | by 6 MPI | by 4 MPI | | by 6 MPI | by 4 MPI | by 4 MPI |
| TH ODU | 0.33 | 0.13 | 0.30 | 0.46 | 0.09 | 0.13 | 0.36 | 0.51 |
| TH-ir | 20,355 | 9,318 | 67,830 | 79,061 | 1,087 | 6,400 | 19,973 | 59,929 |
| Cells (%) | (23.5%) | (16.1%) | (25.5%) | (24.0%) | (7.5%) | (15.0%) | (10.0%) | (10.2%) |
| hKi-67-ir | 352 | 0 | 1,858 | 3,412 | 0 | 532 | 1,038 | 2,402 |
| cells (%) | (0.6%) | (0.0%) | (0.6%) | (1.2%) | (0.0%) | (1.2%) | (0.4%) | (0.4%) |
| 5-HT-ir | n.d. | n.d. | n.d. | n.d. | n.d. | n.d. | n.d. | 277 |
| cells (%) | | | | | | | | (0.04%) |

Normalized TH ODU values range from a minimum of 0, representing the denervated striatum, to 1, representing the intact striatum;
(%) = median % of estimated hNuclei-ir cells;
MPI = months post-injection;
n.d. = not determined

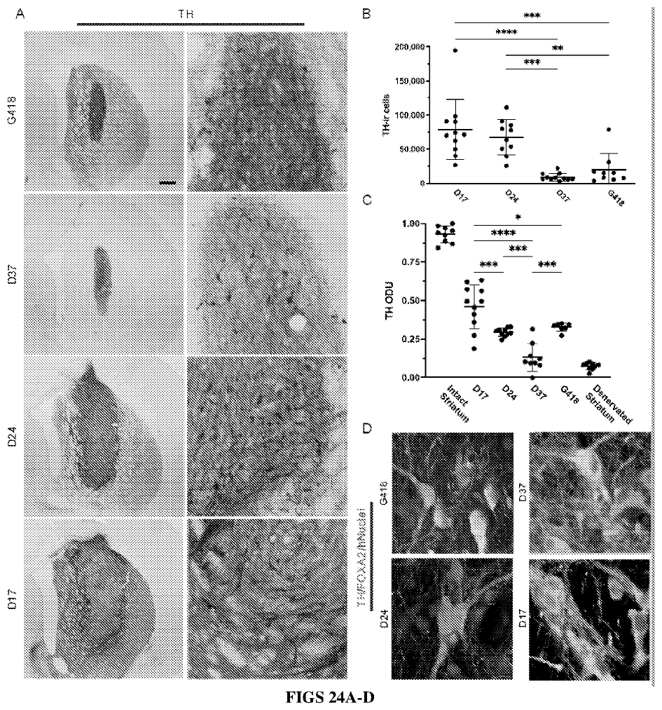

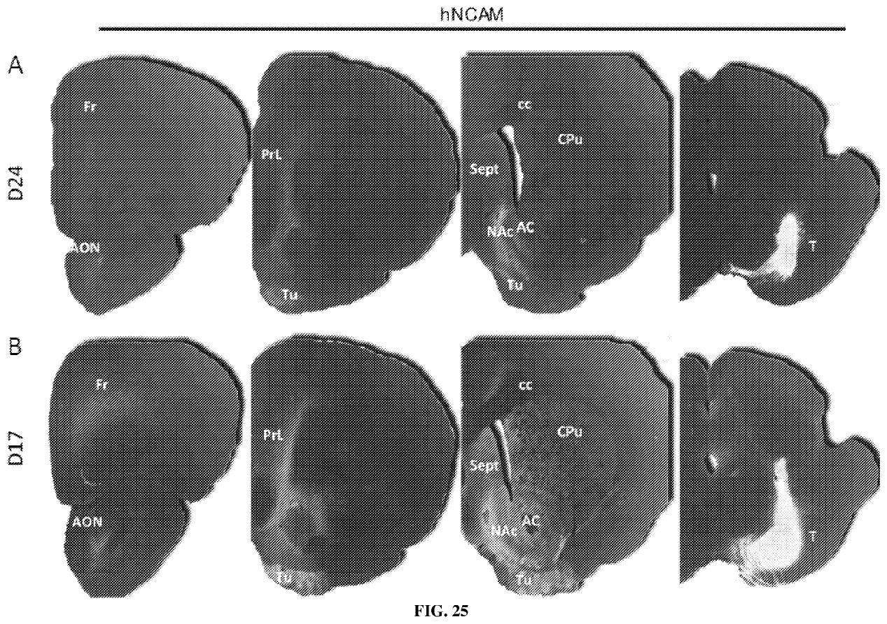

To demonstrate the functional capacity of each cell type, d-amphetamine-induced rotation testing at baseline (10-11 weeks after 6-OHDA lesioning) was performed at 2, 4, and 6 months post-transplantation (FIG. 24A). Hemiparkinsonian rats that received vehicle or D37 grafts failed to demonstrate functional recovery. A mixed-effects ANOVA with Tukey's post-hoc testing revealed that rats receiving D17. D24, or G418 cells exhibited significant (P<0.005, P<0.005, P<0.05) recovery of motor asymmetry by 6 months post-injection. Additionally, animals receiving D17 grafts displayed full (P<0.0005) normalization of rotations by 4 months post-injection. These surprising results demonstrate the superiority of the D17 cells to promote functional recovery in vivo, as observed using an animal model of PD.

To quantify survival of transplants of each cell type, human-specific nuclei (hNuclei) were counted in graft sections using unbiased stereology (FIG. 23B, FIG. 23D). An average (+SD) of 304.303±140,487 hNuclei-ir cells in the D17 group; 266,956±95,419 in the D24 group; 52,623±22,955 in the D37 group; and 108,093±188,944 in the G418 group were estimated based on these experiments, representing 67.6%, 59.3%, 11.7%, and 24.0%, respectively, of transplanted cells. A one-way ANOVA with Tukey's post-hoc adjustment demonstrated better engraftment and survival of grafts comprised of D17 (P<0.005, P<0.01) and D24 (P<0.005, P<0.05) cells compared to D37 and G418, respectively.

Excessive levels of proliferation would preclude any cell type from clinical use due to the increased risk for developing intracerebral teratomas or overgrowth of lineage restricted cells (e.g. neural progenitors). Stereological estimates of human-specific Ki-67 (hKi-67) revealed a median (±IQR) of 3,412±1,391 hKi-67-ir cells in D17 grafts; 1,858±2,275 in D24 grafts; 0±180 in D37 grafts; and 352±697 in G418 grafts, representing only 1.2%, 0.6%, 0.0%, and 0.6% of hNuclei-ir cells, respectively (FIG. 23C, FIG. 23E). We detected significant differences between total number of hKi-67-ir cells for D17 and G418 (P<0.01) or D37 (P<0.05) as well as between D24 and D37 (P<0.05) and between D17 and D37 (P<0.005) for the number as a proportion of hNuclei-ir cells using a Kruskal-Wallis rank sum test with Dwass-Steele-Critchlow-Fligner post-hoc test. These findings demonstrate that grafts of cells transplanted earlier in differentiation contained more proliferating cells post-implantation. D17 and D24 grafts were qualitatively similar in size at 3 months and 6 months, suggesting that any volumetric expansion related to proliferation subsided soon after transplant. Critically, there was no evidence of teratomas or outgrowths compressing neighboring brain regions.

Stereology was used to estimate the number of TH-ir cells in each graft and an average (±SD) of 79,061±44,167 TH-ir cells in D17 grafts; 67,830±25,944 in D24 grafts; 9,318±5,523 in D37 grafts, and 20,355±23,452 in G418 grafts was observed, representing 24.0%, 25.5%, 16.1%, and 23.5% of estimated hNuclei-ir cells, respectively (FIGS. 24A-B). The TH-ir population was significantly larger in D17 (P<0.0001, P<0.005) and D24 (P<0.0005 and P<0.01) transplants compared to D37 and G418 transplants, respectively, by one-way ANOVA with Tukey's post-hoc test. There was also a significant difference between D17 and D37 (P<0.05) for TH-ir cell yield.

To evaluate the ability of each cell type to reinnervate the host striatum with TH-ir axons, the inventors measured TH optical density in the striatum, excluding the body of the graft. Using the TH-denervated striatum of vehicle-treated animals and the contralateral intact striatum as reference points, the data were rescaled from 0 to 1 based on the minimum and maximum values obtained, respectively, and converted to optical density units (ODU) (FIG. 24C). The inventors calculated a mean (±SD) of 0.46±0.14 ODU in D17-treated animals; 0.29±0.03 ODU in D24-treated rats; 0.13±0.09 ODU in D37-treated rats; and 0.33±0.03 ODU in G418-treated rats. The D17 grafts had significantly more TH-ir processes than any other cell type (P<0.0005, P<0.0001, P<0.05 compared to D24, D37, and G418, respectively), while both D24 (P<0.001) and G418 (P<0.0005) cells had significantly more than D37 transplants, as shown using a one-way ANOVA with Tukey's post-hoc adjustment. Together, these data demonstrate that cells transplanted earlier in development (namely D17) comprise populations enriched for TH and neurite outgrowth.

FOXA2 plays a critical role in the induction and maintenance of authentic mDA neurons (Domanskyi, Alter, Vogt, Gass, & Vinnikov, 2014; Kittappa, Chang, Awatramani, & Mckay, 2007). Immunofluorescent co-labeling was utilized to determine FOXA2 expression in hNuclei/TH-ir neurons (FIG. 24D) and showed that most transplanted cells expressed FOXA2. A substantial subset of hNuclei/FOXA2-ir cells also expressed TH, confirming an authentic mDA phenotype.

### Example 15

### Long-Range Site-Specific Innervation by mDA Precursor Cells

The ability to innervate over long distances is extremely helpful for promoting therapeutic responses from administering stem cell grafting to treat PD in the human brain. To assess these capabilities in the cells, the inventor grafted D17 cells or D24 cells into the SN of rats and examined whether long-range projections to their natural targets in the forebrain were formed. At 6 months post-grafting, hNCAM immunoreactivity was evaluated in coronal sections to identify fibers emanating from the grafts and their targets (FIG. 25). Projections from D24 grafts primarily innervated A10 structures in the prelimbic cortex, olfactory tubercle, anterior olfactory nucleus, septum, and nucleus accumbens, with sparse fibers in the striatum, an A9 target. The inventors observed markedly denser innervation of these same A9 and A10 targets in addition to the frontal cortex (A10) by D17 grafts. In both D17- and D24-grafted animals, we observed hNCAM-ir fibers in the most rostral brain regions examined (approximately 7-8 mm from the most rostral aspect of the graft in the SN), demonstrating the ability to project fibers over long-distances. These results demonstrate the superiority of D17 cells in innervating their natural targets over long distances.

### Example 16

### Dose Response of mDA Precursor Cells

The D17 grafts demonstrated the most robust efficacy, viability, and dopaminergic phenotypic expression without problematic proliferation, and were chosen by the inventors for further study. To determine an optimal dosing strategy, the concentration of D17 cells were titrated down from the amount used in the initial examination. Hemiparkinsonian athymic rats received 3 μL striatal transplants of the maximum feasible dose (MFD) of 150,000 cells/μL, High dose (40,000 cells/μL), Medium dose (10,000 cells/μL), Low dose (2,500 cells/μL), or vehicle control (n=8-11/group). Motor asymmetry was assessed every 2 months post-transplantation by d-amphetamine-induced rotations for 6 months, at which point rats were sacrificed and brains were assessed histologically.

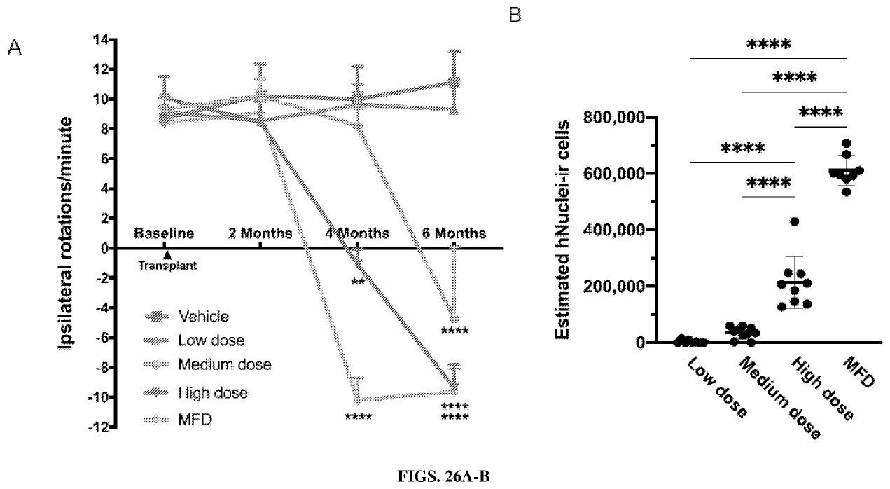

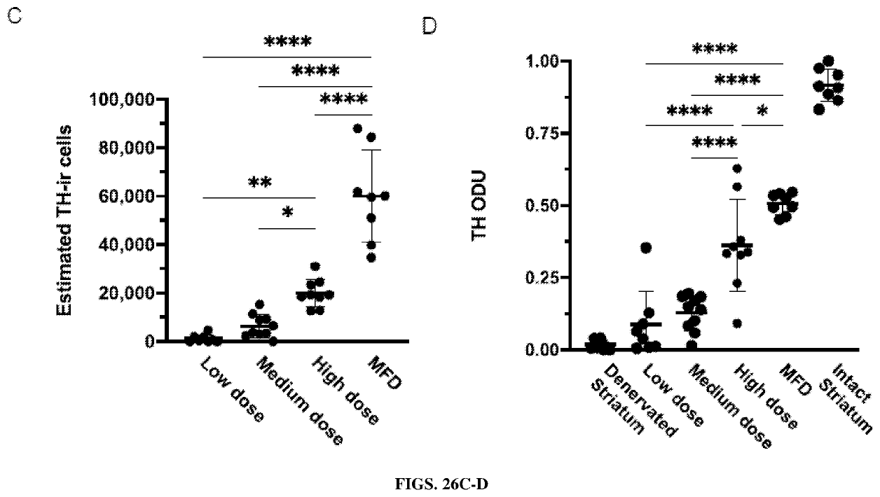

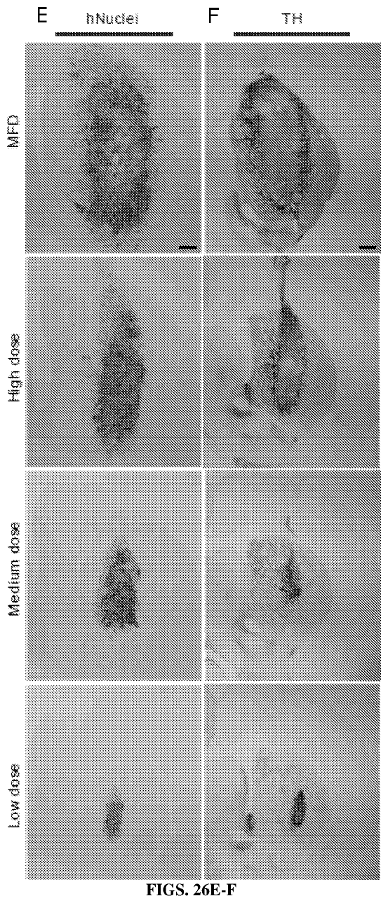

The inventors observed a clear dose response in all behavioral and histological analyses. Rats that received vehicle or low dose of transplanted cells failed to demonstrate functional recovery in the d-amphetamine-induced rotation test. A mixed-effects ANOVA with Tukey's post-hoc adjustment revealed that rats that received the medium (P=0.002), high (P<0.0001), or ‘maximum feasible’ (P<0.0001) dose displayed full normalization of motor asymmetry by 6 months after transplantation (FIG. 26A). Notably, grafts of the high (P=0.0002) or ‘maximum feasible’ (P<0.0001) dose were effective in normalizing rotations as early as 4 months post-injection. Further, the extensive innervation in rats from the two highest dose groups resulted in over-compensation of d-amphetamine-induced rotation resulting in circling in the direction opposite to what was seen pre-grafting (FIG. 26A).

When hNuclei staining in the grafts was quantified (FIG. 26B, FIG. 26E), the number of surviving cells directly correlated with dosage, with an estimated mean (±SD) 611,588±53,377 surviving cells in MFD-treated animals; 214,898±91,906 in high dose animals; 36,848±18,816 in medium dose animals; and 4,604±5,904 in low dose animals. Significant differences were calculated by a one-way ANOVA with Tukey's post-hoc test for MFD compared to low, medium, and high doses as well as high compared to medium and low doses (P<0.0001 for all comparisons).

We also quantified the number of TH-ir cells within each graft (FIG. 26C, FIG. 26F) using unbiased stereology. As expected, the number of TH-ir cells directly correlated with dosage, with an estimated mean (±SD) 59,929±18,927 TH-ir cells in MFD grafts; 19,973±5,759 in high dose grafts; 6,400±4,709 in medium dose grafts; and 1,087±1,471 TH-ir cells in low dose grafts, representing 10.2%, 10.0%, 15.0%, and 7.5% of estimated hNuclei-ir cells, respectively. Significant differences were calculated for MFD (P<0.0001) compared to low, medium, and high doses as well as high compared to medium (P=0.03) and low (P=0.002) doses using a one-way ANOVA with Tukey's post-hoc adjustment.

In order to evaluate the ability for each cell type to replenish the host tissue with TH-ir processes, the inventors measured and processed TH optical density in the striatum in the same fashion as described above. The density of projections reinnervating the striatum correlated with dosage, with a mean (±SD) of 0.51±0.04 ODU. 0.36±0.16 ODU. 0.13±0.06 ODU, and 0.09±0.12 ODU calculated in the MFD, high, medium, and low dose groups, respectively (FIG. 26D). Significant differences were found when comparing MFD to low (P<0.0001), medium (P<0.0001), and high (P<0.05) doses, as well as for high dose compared to medium (P<0.0001) and low (P<0.0001) doses with a one-way ANOVA and Tukey's post-hoc test.

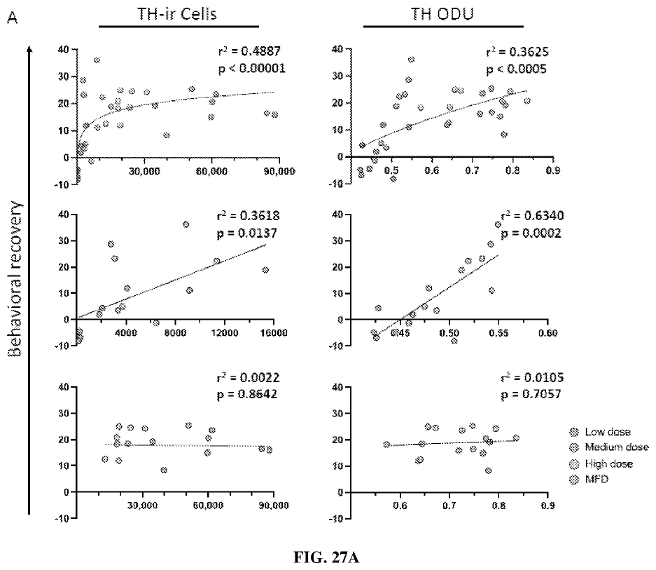

Upon first assessment, the low dose group displayed no behavioral correction despite containing 4,604±5,904 hNuclei-ir cells and 1,087±1,471 TH-ir cells. Further inspection revealed 5 rats with little-to-no surviving grafts that did not recover motor asymmetry. In contrast, rats with substantial surviving grafts (containing 1,827; 2,068; and 4,100 TH-ir cells) recovered to varying degrees (18%; 49%; and 85% reduction in rotations, respectively) by 6 months post-transplantation. To further scrutinize the behavioral effect of different doses of D17 mDA progenitors, behavioral recovery was plotted against number of TH-ir cells and TH optical density (FIG. 27A). In view of the non-linear quality of the data, logarithmic regression was used to assess these correlations. Results of r2=0.3625 (P<0.0005) for TH optical density and r2=0.4887 (P<0.00001) for TH-ir cells were observed, indicating moderate correlations with functional recovery. When we partitioned the data into low/medium and high/MFD groups, linear relationships in the low/medium groups for TH optical density (r2=0.6340; P<0.0005) and TH-ir cells (r2=0.3618; P<0.05) were observed. These analyses indicated that while there was a clear ceiling effect for both measures of dopaminergic phenotype, graft-derived innervation was a more robust indicator of overall graft function at lower doses.

### Example 17

### Characterization of mDA Phenotype In Vivo

To confirm mDA phenotype, immunofluorescent triple-labeling of grafts at 6 months post-injection experiments were performed (FIG. 27B). A majority of grafted cells expressed TH/FOXA2 with most TH-co-expressing cells localized to the borders of the graft. Additionally, many hNuclei-ir cells expressing TH/GIRK2 (62.6±2.9%) were observed, with a smaller population of TH/Calbindin-ir (31.8±1.7%) cells (FIG. 27C), evincing both A9 and A10 dopaminergic subtypes, consistent with the long-range innervation patterns by D17 cells grafted to the SN. Some GIRK2-ir cells were observed that did not express TH (3.3±1.2%), which may be of parabrachial or paranigral origin. These results support the observations that D17 cells produced superior innervation of long-range targets as compared to other cells.

### Example 18

### Testing for Proliferation, Gliosis, or Serotonergic Contamination

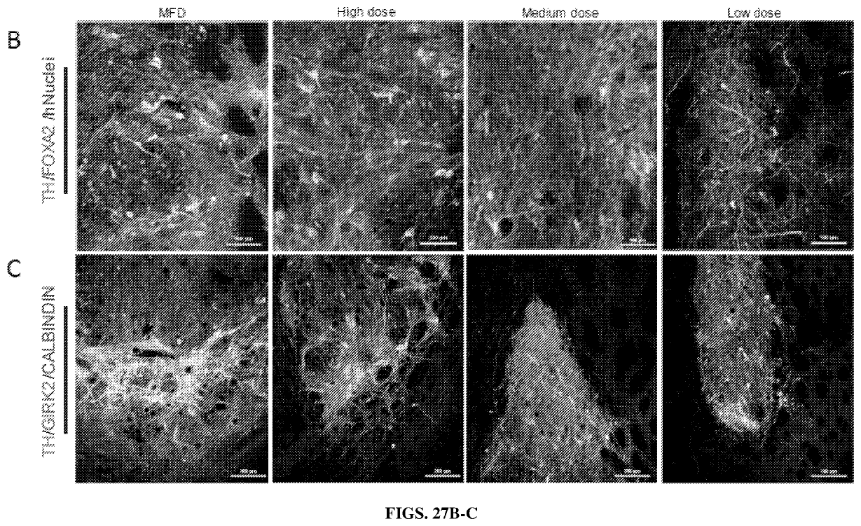

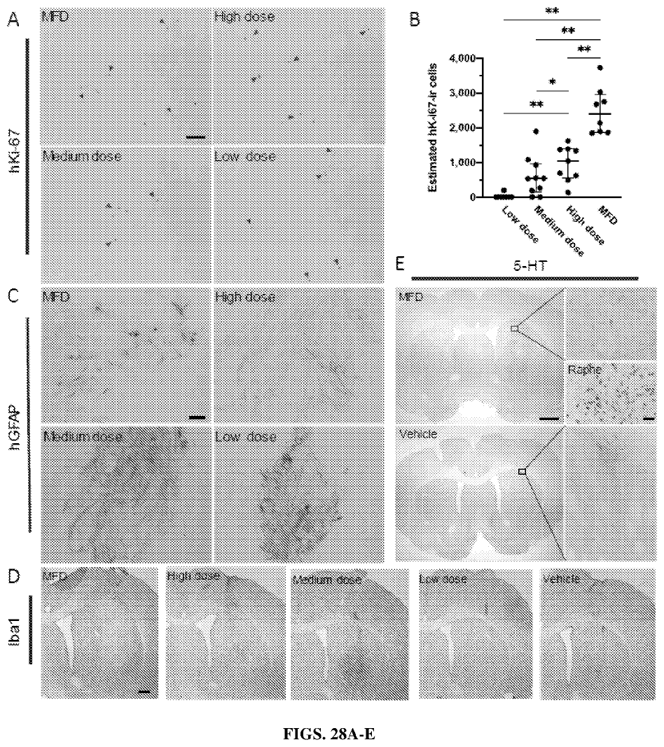

Critically, low levels of continued proliferation were seen in the grafts after 6 months, as determined via unbiased stereology performed on sections stained for hKi-67 (FIG. 28A, B) and in agreement with our previous studies. We estimated 2,402±1,006 hKi-67-ir cells in MFD grafts; 1,038±741 in high dose grafts; 532±745 in medium dose grafts; and 0±5 hKi-67-ir cells in low dose grafts, representing 0.4%, 0.4%, 1.2%, and 0.0% of estimated hNuclei-ir cells, respectively. We calculated significant differences for MFD compared to high (P=0.003), medium (P=0.004), and low (P=0.003) dose as well as low compared to high (P=0.003) and medium (P=0.04) dose groups for total number of hKi-67-ir cells and for percentages of low compared to high and ‘maximum feasible’ dose (P<0.05) using Kruskal-Wallis and Dwass, Steel, Critchlow-Fligner method. Again, we report no evidence of teratoma formation.

To assess the degree of astrocytosis within the grafts, sections were stained with human-specific GFAP (FIG. 28C). We observed patterns of immunoreactivity, largely resembling long fibers coursing through the body of the graft with some astrocytic bodies, consistent with the GLAST expression detected by qPCR and similar to murine fVM grafts (L. H. Thompson, Kirik, & Bjorklund, 2008). We evaluated Iba1-ir to determine whether there was an elevated microglial response to the xenotransplants. Generally Iba1-ir was not pronounced, except near the injection site in the cortex in close proximity to the craniotomy, site of dura puncture and near the periphery of the graft, where animals did show slightly increased immunoreactivity and/or activated microglia. Iba1-ir microglia with reactive morphology were observed within or near the perimeter of the transplants (and one animal in the medium dose group had more intense Iba1-ir in the graft), and some animals had a population of microglia with thickened processes and more intense staining near the dorsal aspect of the grafts in close proximity to the craniotomy and site of dura puncture (FIG. 28D). We found that D17 grafts contained very few serotonergic (5-HT) cells (FIG. 28E), with an estimated 277±194 5-HT-ir cells (0.04% of estimated hNuclei-ir cells) in MFD grafts. These data collectively show an overall lack of outgrowth of off-target cell types or host gliosis.

### Example 19

iPSC-Derived mDA Precursor Cells for Treatment of PD

As shown in the above experiment, grafts of mature (D37/G418) neurons clearly differed from transplants of immature neurons (D24) and progenitors (D17), both in terms of behavioral effects and regarding histological characteristics. The difference in graft size was apparent as early as 3 months post-injection based on hNCAM- and TH-immunostaining, with mature (D37/G418) neurons forming thin, pencil-shaped grafts, and younger (D17/D24) cells forming comparably large grafts. At 6 months post-grafting, we observed a robust dopaminergic phenotype in D17 and D24 grafts in comparison to D37 or G418, which was also reflected by a full reversal of d-amphetamine-induced motor asymmetry in D17- and D24-grafted rats. For all cell types and doses, grafted cells expressed TH/FOXA2, confirming that the maturation during the 6 months after grafting in vivo continued and resulted in mature mDA neurons derived from the implanted progenitors and immature neurons. When we grafted D17 and D24 cells intranigrally, preferential innervation of both A9 and A10 targets over long distances was observed. These findings are consistent with earlier observations with fVM and ESC-VM tissue (Cardoso et al., 2018; Grealish et al., 2014) and are also supported by experiments above showing both TH/GIRK2-ir and TH/Calbindin-ir cells within the grafts. GIRK2 and CALBINDIN are commonly used to differentiate A9 and A10 mDAs; sequencing and/or advanced multiplexing may also be used to further define these populations. The ability of the grafted iPSC mDA cells to project fibers over long distances in the rat brain indicates that these approaches can be applied to the human putamen.

Marked differences in outgrowth of graft-derived TH-immunoreactive fibers into the host striatum were observed depending on the differentiation protocol used. Rats grafted with D17, D24, and G418 cells exhibited TH-ir fibers covering the whole striatum, while rats grafted with D37 cells, which exhibited no graft-induced behavioral recovery, showed almost no graft derived TH-ir axons innervating the host. In fact, high magnification images revealed that though these grafts contained TH-ir cells and fibers, their axons ended abruptly upon reaching the outermost edges of the D37 grafts. With comparable numbers of TH-ir cells in D17 and D24 grafts as well as in D37 and G418 grafts, it is highly likely that the propensity for D17 and G418 cells to innervate the host underlies their function. Indeed, similar behavioral outcomes in animals with large (67,800 TH-ir cells) D24 grafts as in animals with smaller (6,400 TH-ir cells) D17 grafts were observed; without wishing to be bound by any theory, it is anticipated that this finding was due to the similar reinnervation of the host striatum. Regression analyses showed a ceiling effect for number of D17 TH-ir cells and their processes but also that, at lower doses, innervation was more highly correlated than number of TH-ir cells with behavioral recovery. These results observed in rats may be particularly important for obtaining improved therapeutic responses in humans, where the putamen (3.96 cm³ in PD patients (Yin et al., 2009)) is substantially larger than the rat striatum. More TH-ir cells and their processes may be necessary to produce clinical benefit in humans; alternately or in combination, cells can be deposited at multiple sites along multiple needle tracts in an arrangement conducive to total reinnervation of the putamen, possibly without the diminishing returns associated with large grafts seen here in rats. The indication here that D17 mDA progenitors are effective across a wide range of doses indicates that clinicians may have some latitude in utilizing various surgical approaches for administration of the cells. Additional studies to even further optimize the dosing regimen in humans can be performed and it is anticipated that similar therapeutic results will be observed.

Proliferating cells in grafts of mature cells (G418, D37) were only rarely observed, consistent with previous observations (Hiller et al., 2020; Wakeman et al., 2017). While D17 and D24 grafts contained more hKi-67-ir cells than grafts of G418/D37 cells, the number of proliferating cells as a proportion of surviving grafted cells was low (<1,000 per 100,000 hNuclei-ir in D17/D24 grafts), demonstrating that a purification step was not necessary to prevent undesirable cell proliferation. Further, hKi-67-ir cells were not present in clusters indicative of active cell division in any of the grafted rats. Another safety concern is the development of GIDS which have been reported in a subset of patients transplanted with fVM (Freed et al., 2001; Hagell & Cenci, 2005; Olanow et al., 2009), and it has been suggested that aberrant grafting of serotonergic neurons contributes to the development of GIDs. As further evidence of the safety of the iPSC-derived mDA precursor cell grafts, the inventors did not observe serotonergic neurons in numbers near those postulated to induce GIDs (Carlsson et al., 2009).

The above transplantation studies using stem cell-derived mDA cells utilize, in some embodiments, progenitor and immature neuron developmental stages. Without wishing to be bound by any theory, it is anticipated that the mDA precursor cells provided herein may exhibit many of the beneficial effects of fetal tissues that have been used successfully in clinical trials (Li & Li, 2021). It is difficult to directly compare the developmental stage of the cells used in different studies due to differences in the differentiation protocols, but many of the cells that are the focus of efforts to adapt them for translational use incorporate exposure to neuronal maturation factors such as BDNF, GDNF, TGF-β3, and/or DAPT (Doi et al., 2020; Kim et al., 2021; Kirkeby et al., 2017; Song et al., 2020). In studies where different developmental stages were directly compared, the conclusion has been that NURR1+ immature neurons were more efficacious than less mature progenitors (Ganat et al., 2012; Qiu et al., 2017). In contrast, the above studies demonstrate that D17 cells exposed to mDA patterning factors (SMAD inhibition, SHH, WNT, FGF8) and cryopreserved prior to NURR1 expression result in grafts that outperform the same cells cultured an additional week with maturation factors (D24, NURR1+/−). Cells at both maturational stages engraft and mature into mDA neurons in similar numbers, suggesting the performance disparity is not simply due to differences in proliferative potential; without wishing to be bound by any theory, it is that this may results from differences in innervation, A9 patterning, and/or other early mDA maturation signals received in vivo. Single-cell sequencing of grafted cells can be used to further analyze other non-dopaminergic cells that are comprised in the grafts. Importantly, D17 cells were observed to have been adequately patterned and did not require exposure to maturation factors before transplantation to “lock in” mDA patterning or prevent the proliferation of undesirable (e.g., serotoninergic) cell types.

Without wishing to be bound by any theory, the data presented above support the idea that if mDA neurons or precursor cells are too mature at the time of grafting to the striatum, they typically survive less well and have less marked behavioral effects. The above studies also demonstrate that relatively small grafts of D17 progenitors can give rise to dopaminergic innervation sufficient to elicit behavioral recovery in hemiparkinsonian rats. These data support the idea that a relatively small total number of cells can be injected at a small number of locations in the striatum in each patient, which may result in therapy of PD and also indicate that favorable clinical safety may be observed.

Some other mDA progenitor cells that are being tested in clinical trials have been derived from ESCs (NCT04802733) (Piao et al., 2021) or iPSCs (JMA-IIA00384, UMIN000033564) (Doi et al., 2020). The above data show that the mDA precursor cells provided herein (e.g., D17 cells) can be administered to a patient to treat PD. If desired, the mDA precursor cells may be administered in combination with an immunosuppression drug or regimen and/or dopamine replacement therapy, if desired. In some embodiments, a dopamine replacement therapy is not administered to the patient after administration of the mDA precursor cells. It is anticipated that the mDA precursor cells provided herein (e.g., D17 cell) when administered to select PD patients can be able to achieve significant clinical benefit using dopamine cell replacement therapy in carefully selected groups of.

### Example 20

### Levels of Off-Target Cell Types

Incorrect patterning during midbrain DA progenitor differentiation can yield dangerous off-target cell types such as neural progenitors with a forebrain (rostral) phenotype and serotonergic cells. Forebrain-type cells can be a particular concern, because previous DA neuron differentiation protocols often included neural progenitors with rostral (FOXG1+) and/or lateral (PAX6+) cell types that can form rosette structures in vivo, resulting in neural outgrowth that has been observed to persist for months post-engraftment (Kriks et al., 2011). Cultures were thus tested for off-target or non-dopaminergic cell types.

FCDI DAPC-1 (Day 17 DA progenitor) cells were differentiated and cryopreserved as described in Example 1 (Table 2). Cells were thawed and washed with DPBS prior to flow cytometry or qPCR analysis of Day 17 progenitor cells (0 days post-thaw, 0DPT). Alternatively, cells were thawed and cultured in DA Maturation Medium (Table 1) for analysis of cells at later time points (7-20 days post-thaw, 7-20DPT) to assess expression of markers expressed in more mature cells.

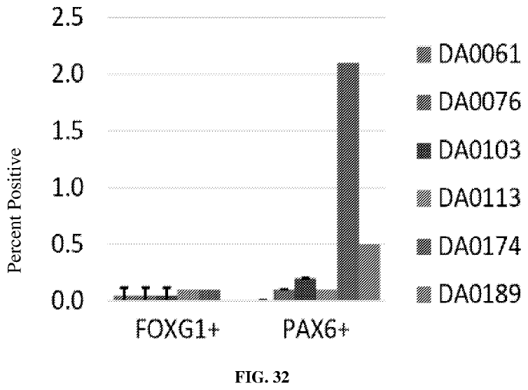

Flow cytometry assays were used to monitor FOXG1 and PAX6 expression at thaw. The FOXG1 and PAX6 flow cytometry assays were performed on 6 representative engineering batches, each thawed one or two separate times (n=9 total). On average, FCDI DAPC-1 is 0.1% FOXG1+ with a standard deviation (SD) of 0.1%, and 0.4% PAX6+ with a SD of 0.7% at thaw confirming that FCDI DAPC-1 lacks expression of markers for these off-target cell types (FIG. 32). Based on flow cytometry assays non-target cell markers FOXG1+ and PAX6+ that would be expressed by potentially dangerous cells, the cell culture contains a very low percentage of such forebrain neuron progenitors. Results are shown below in Table 5.

TABLE 5

| Flow Cytometry | Average (% positive) ± |
|---|---|
| Assay | Standard Deviation |
| FOXG1 | 0.1 ± 0.1 |
| PAX6 | 0.4 ± 0.7 |

In substantial quantities, inclusion of serotonergic cells in grafts can be potentially dangerous and may contribute to graft-induced dyskinesias (Carlsson et al., 2009). Definitive markers for serotonergic cells include serotonin (5-HT) and tryptophan hydroxylase-2 (TPH2) which is the rate limiting enzyme in 5-HT synthesis, and 5-HT transporter (SERT). Since these markers are only expressed in mature cells, assays were not performed on FCDI DAPC-1 immediately post-thaw (ODPT). There are no known definitive markers for serotinergic cell progenitors. To determine the earliest time point at which serotonergic cells could be detected, FCDI DAPC-1 were evaluated at 0DPT (zero days post-thaw), 7DPT, 14DPT, and 19-20DPT using qPCR and immunohistochemistry (ICC). As a positive control for serotonergic marker expression by qPCR, a total-RNA sample from human Pons, a brain region harboring serotonergic cells, was included.

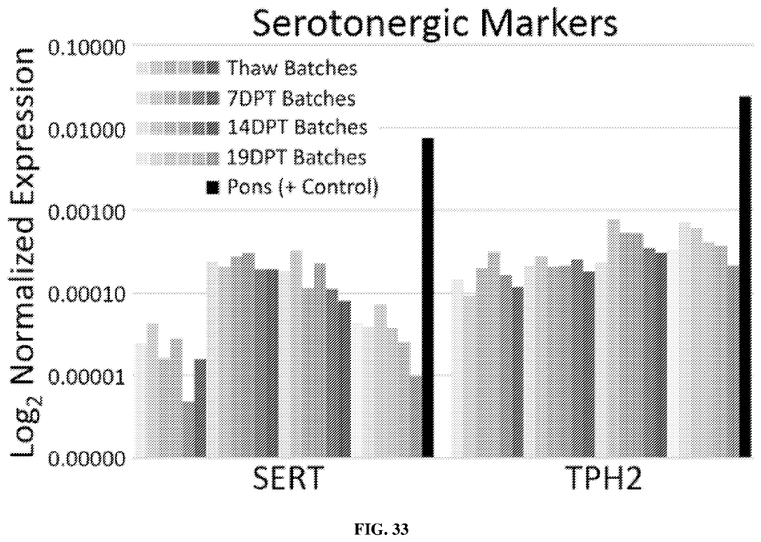

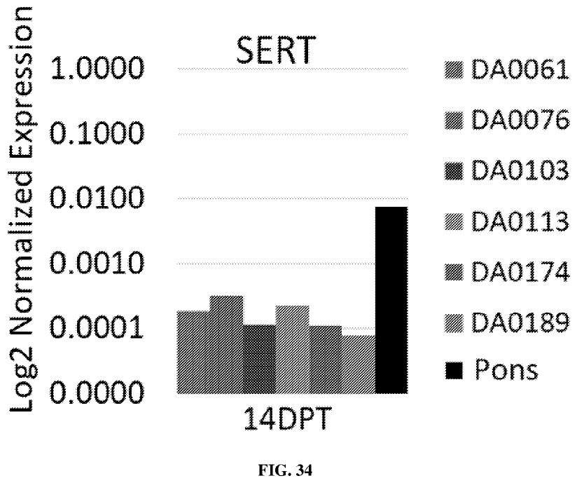

Expression levels of SERT and TPH2 were observed to be low in FCDI DAPC-1 both at 0DPT and throughout maturation in culture compared to Pons (FIG. 33). Results are shown below in Table 6. SERT expression increases significantly between 0DPT and 7DPT, and then was markedly reduced after 14DPT. TPH2 showed a gradual increase of expression from 0DPT to 19DPT. For both markers, peak expression is seen at either 7DPT or 14DPT, and expression at 14DPT is consistent across different DAPC-1 batches (FIG. 34). Results are shown below in Table 7.

TABLE 6

| | Time- | Average ΔCq ± |
|---|---|---|
| Assay | point | Standard Deviation |
| SERT | Thaw | 15.96 ± 1.12 |
| | 7DPT | 12.10 ± 0.29 |
| | 14DPT | 12.68 ± 0.77 |
| | 19DPT | 14.60 ± 0.59 |
| | Pons | 7.06 ± N/A |
| TPH2 | Thaw | 12.65 ± 0.63 |
| | 7DPT | 12.15 ± 0.22 |
| | 14DPT | 11.48 ± 1.15 |
| | 19DPT | 11.07 ± 0.47 |
| | Pons | 5.39 ± N/A |

TABLE 7

| qPCR | Average ΔCq ± | Pons Average |
|---|---|---|
| Assay | Standard Deviation | ΔCq |
| SERT | 12.68 ± 0.77 | 7.06 |
| TPH2 | 12.65 ± 0.63 | 5.39 |

Log₂ normalized expression values below 0.001 to be markers were considered to be very low expression, and values below 0.0001 were considered to be below detection. These results indicate that cells in FCDI DAPC-1 cultures express serotonergic markers, SERT and TPH2, at a very low level as shown using qPCR.

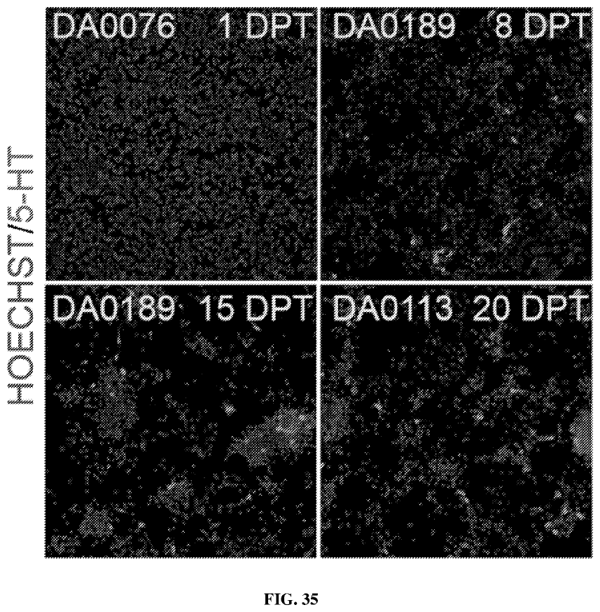

To determine the percent of serotonergic cells, we performed ICC staining for 5-HT on cells along the post thaw culture timecourse (FIG. 35). Results are shown below in Table 8. Essentially no 5-HT+ cells were observed at 1 day post-thaw (1DPT). A significant population of 5-HT+ cells was observed at 8DPT, 15DPT, and 20DPT. Quantification using high content imaging software (Molecular Devices ImageXpress) showed the percentage of serotonergic neurons to be approximately 0.2% (0DPT), 3.4% (8DPT), 2.1% (15DPT, and 5.6% (20DPT). This data demonstrates that DAPC-1 contains approximately 5% serotonergic neuron progenitors that can be visualized by 8DPT as mature (post-mitotic) serotonergic neurons. This percentage does not increase over time.

TABLE 8

| | Number of | Average | Average |
|---|---|---|---|
| | Wells Counted | Nuclei | 5-HT+ (%) |
| DAPC-1 | 6 | 1842 | 0.17 |
| 7DPT | 8 | 1509 | 3.43 |
| 14DPT | 8 | 670 | 2.1 |
| 20PT | 12 | 955 | 5.57 |

### Example 21

### Materials and Methods

The following materials and methods were used in Examples 13-20.

Statistical analysis: Statistical analysis was performed in SAS (for stereological and behavioral outcomes in dosing study) or Prism (version 9.1.2, GraphPad). Graphs were made in Prism. Data from immunohistochemical analyses were analyzed using a one-way analysis of variance with Tukey's test post hoc test, except for hKi-67 which was analyzed by Kruskal-Wallis test with Dwass, Steel, Critchlow-Fligner method post-hoc. Behavioral data were analyzed by mixed effects analysis of variance with Tukey's test post hoc test. Histological data were represented as mean±SD except for hKi-67 (median±IQR). Median percentages were reported for TH- or hKi-67-ir cells as proportion of hNuclei-ir cells for each animal. Rotations were reported as mean±SEM.

Cell differentiation: Research use G418 neurons (iCell DopaNeurons, Fujifilm Cellular Dynamics, Inc.) were derived as previously described (Hiller et al., 2020; Wakeman et al., 2017), utilizing an engineered iPSC line to allow G418 drug selection of neurons during the differentiation process, and with cryopreservation of the neurons on process day 38. For clinical development, a non-engineered iPSC line that had been reprogrammed using procedures and reagents appropriate for cell therapy development was selected and expanded into a master cell bank (MCB) and working cell bank (WCB) in a cGMP manufacturing facility (Waisman Biomanufacturing, Madison, WI). The iPSC-mDA differentiation protocol was adjusted for this iPSC line, including simplification of SMAD signaling inhibition (LDN-193189, Reprocell) and shifting GSK-3 inhibition (CHIR99021, Reprocell) one day later, to process day 2, at a higher concentration adjusted for this timing. Raw materials were upgraded to be appropriate for clinical development, including the use of GMP grade Shh C25II, BDNF, GDNF, and TGFβ3 (Bio-techne). D37 neurons were purified in-process using mitomycin C (Tocris, 150 ng/mL on process days 27 and 29) as previously described (Hiller et al., 2020), and were cryopreserved with CryoStor (Biolife Solutions) on process day 37. D17 progenitors were manufactured using the same differentiation process, except that progenitor aggregates were dissociated with CTS TrypLE Select Enzyme (Thermo) and cryopreserved on process day 17, without being exposed to maturation medium (Kriks et al., 2011) or mitomycin C treatment. D24 immature neurons were cryopreserved later in the process (process day 24), after being plated in maturation medium for one week, but without mitomycin C treatment. The cells used to compare iPSC DA maturation stages were produced in a research lab using the manufacturing process adapted for clinical translation. The D17 cells used for the dose-ranging study were made in a controlled, non-classified clean lab using the same process.

qPCR: Cells were thawed and lysed with Buffer RLT Plus (Qiagen) containing 1:100 beta-Mercaptoethanol. Total RNA was extracted using a RNeasy Plus kit (Qiagen). cDNA was generated using a High Capacity RNA-to-cDNA Kit (ThermoFisher) with a 500 ng RNA input. Quantitative polymerase chain reaction (qPCR) was performed on a LightCycler480 (Roche) using TaqMan Gene Expression Master Mix (ThermoFisher), TaqMan assays (see Table 5 for list of assays), and 2.5 ng cDNA input. Values are expressed as relative to glyceraldehyde-3-phosphate dehydrogenase (GAPDH). Three biological replicates were analyzed in technical triplicates for each time point.

Flow cytometry: Cells were thawed as previously described (Wakeman et al., 2017). Cells were centrifuged and stained with GhostDye510 (Tonbo Biosciences), fixed with 4% formaldehyde, and washed with wash buffer (2% FBS in DPBS). Cells were stained with primary antibodies in 1×BD Perm/Wash (BD Biosciences)+0.2% Triton X-100 (except for Map2 stain, which did not contain Triton X-100) at 4° C. (see Table 6 for list of antibodies and dilutions), and labeled with secondary antibodies (where applicable) at room temp. Flow cytometry was performed on a MACSQuant® Analyzer 10 flow cytometer (Miltenyi Biotec). Three biological replicates were analyzed for each maturation time point.

Immunocytochemistry: Cells were thawed, seeded at 170,000 cells/well to 96-well plates, cultured overnight, and fixed with 4% formaldehyde. Cells were stained with primary antibodies in stain buffer (2% FBS, 2% Donkey Serum, 0.2% Triton X-100 in DPBS) at 4ºC (see Table 6 for list of antibodies and dilutions), and labeled with secondary antibodies (where applicable) and Hoechst (ThermoFisher) at room temp. Cells were analyzed on an ImageXpress High Content Imager (Molecular Devices) at 10× magnification. Three biological replicates were analyzed for each time point.

Animal procedures: All animal procedures were performed with Institutional Animal Care and Use Committee approval from Rush University Medical Center.

Lesion induction and transplant: Female athymic nude (rnu) rats were acclimated for one week following reception. At 9-10 weeks of age, (170-200 g) rats received unilateral injections of 6-OHDA (15 mg in 3 μL 0.5% ascorbic acid) to the right MFB (Anterior/Posterior [AP]: −4.0 mm; Medial/Lateral [ML]: −1.3 mm from bregma, Dorsal/Ventral [DV]: −7.0 mm from dura). Animals with confirmed lesions by 10 weeks post-lesion received striatal (AP: +0.5 mm; ML: +3.0 mm from bregma, DV: −5.3 mm from dura) injections of iPSC-mDA cells (n=8-11/group) and were sacrificed at 3 or 6 months post-transplantation. Cryopreserved cells were thawed and cells counted via trypan blue exclusion. The cells were centrifuged and resuspended at the appropriate densities for injection. Intranigral grafts were placed at AP: +0.5 mm; ML: −3.0 mm from bregma, DV: −5.0 mm from dura. In all experiments, injection volume was 3 μL. A concentration of 150,000 cells/μL was used in the cellular maturity comparison and intranigral experiments, and 2,500, 10,000, 30,000, or 150,000 cells/μL were used for the dose-ranging experiment.

d-amphetamine-induced rotations: Animals received intraperitoneal injections of d-amphetamine (2.5 mg/kg, Sigma), placed in harnesses in semi-opaque chambers, and connected to a Rotometer system (San Diego Instruments). Net ipsilateral (clockwise) rotations for the time period 10-40 minutes following d-amphetamine administration were reported.

Tissue processing: Tissue was processed and immunohistological and stereological analyses were performed as previously described (Hiller et al., 2020). Briefly, rats were anesthetized with a ketamine/xylazine mixture and perfused with normal saline followed by 4% paraformaldehyde. Brains were removed, placed in a sucrose gradient, and sectioned at 40 μM on a sliding microtome. Free-floating sections were stained using antibody concentrations for immunofluorescent triple-labeling or DAB-processing listed in Table 6. Sections were mounted on glass gelatin-coated slides, coverslipped, and imaged.

Stereology: Coverslipped slides were analyzed by unbiased stereology (StereoInvestigator v10.40, MBF biosciences). For cellular maturity comparison experiment, 5.22% of total graft area was probed for TH, hNuclei, or hKi-67 in half series ( 1/12 serial sections) of stained tissue. For dose-ranging experiment, 5.22% of TH-ir and hNuclei-ir grafts, 28.4% of hKi-67-ir grafts, or 20.3% of 5-HT-ir grafts were probed in half series ( 1/12 serial sections) of stained tissue. For animals in low or medium dose groups with Gundersen m=1 coefficient of error ≥0.45 or where no cells were counted in either hNuclei- or TH-stained sections, an additional half series ( 1/12 serial sections) was stained and re-probed using the same parameters. Estimates were then calculated for the full series (⅙ serial sections) and the results were averaged.

Optical density: Grayscale images of 7 (center of graft±3) coronal sections stained for TH were analyzed for each animal. In each section, a contour was drawn around the striatum, excluding the body of the graft, and mean pixel intensity of the area was recorded using ImageJ. Values were averaged for each animal and the data were rescaled considering the minimum point of the denervated striatum as 0 and the maximum point of the intact striatum as 1. Data sets for cellular maturity comparison and dose ranging experiments were rescaled separately.

mDA subtype quantification: Graft sections from 4 MFD animals were stained for TH/GIRK2/CALBINDIN and imaged by a Nikon Eclipse Ti2 confocal microscope with a Nikon A1RHD camera using NIS Elements AR software (version 5.10.01) and stored as .tiff files. Markers in 53-80 cells in each graft were quantified from z-stacks using ImageJ (version 1.53a).

qPCR assay for serotonergic cell population from 0-19DPT: RT-QPCR assays for SERT and TPH2 on 6 FCDI DAPC-1 batches at thaw and in culture for 7-, 14-, or 19-DPT. Each shade represents a different batch at the respective timepoint. Pons is a positive control brain region. Average ΔCq (Cq_{ASSAY}−Cq_{GAPDH}) and standard deviation for each assay among the 6 batches shown in table.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
WO2018/035214
U.S. Pat. No. 5,843,780
U.S. Pat. No. 6,200,806
U.S. Pat. No. 6,833,269
U.S. Pat. No. 7,029,913
U.S. Patent Publn. 2002/0168766
U.S. Patent Publn. 2003/0022367
U.S. Patent Publn. 2003/0211603
U.S. Patent Publn. 20030087919
U.S. Patent Publn. 20030125344
U.S. Patent Publn. 20040002507
U.S. Patent Publn. 20040002508
U.S. Patent Publn. 20040014755
U.S. Patent Publn. 20050192304
U.S. Patent Publn. 20050209261
U.S. Patent Publn. 2007/0116680
U.S. Patent Publn. 2007/0238170
U.S. Patent Publn. 2008/0171385
U.S. Patent Publn. 2011/0229441
U.S. Patent Publn. 2012/0276063
U.S. Patent Publn. 2015/0010514
U.S. Patent Publn. 2015/0265652
U.S. Patent Publn. 2016/0177260
U.S. patent application Ser. No. 13/054,022
U.S. patent application Ser. No. 14/664,245
U.S. patent application Ser. No. 14/830,162
PCT/US2010/024487
PCT/US2011/046796
WO 2009/149233
WO 2010/141801
WO2013/067362
WO 1998/30679
WO 2001/088100
WO 2002/076976
WO 2003/059913
WO 2003/062225
WO 2003/062227
WO 2004/039796
WO 2005/080554
WO 2005/123902
WO 2013/067362
Amit et al., 2000.
Andrews et al., In: Teratocarcinomas and Embryonic Stem Cells, Robertson (Ed.), IRL Press, 207-246,1987.
Animal Cell Culture, 1987.
Bottenstein and Sato, Proc. Natl. Acad. Sci. USA, 76:514-517, 1979.
Byrne et al., Nature, 450(7169):497-502, 2007.
Barker et al., Lancet Neurol. 12(1):84-91, 2013.
Chen et al., Cell, 133:1106-1117, 2008.
Chen et al., Nature Methods 8:424-429, 2011.
Chung, et al., ES cell-derived renewable and functional midbrain dopaminergic progenitors, Proc Natl Acad Sci USA. 108(23):9703-8, 2011.
Current Protocols in Molecular Biology and Short Protocols in Molecular Biology, 1987 and 1995.
Doe et al., J. Pharmacol. Exp. Ther., 32:89-98, 2007.
Embryonic Stem Cell Differentiation in vitro, 1993.
Evans et. al., Nature, 292:154, 1981.
Fernandes, et al., J. Biotechnology, 132(2):227-236, 2007.
Gene Targeting, A Practical Approach, IRL Press at Oxford University Press, 1993.
Gene Transfer Vectors for Mammalian Cells, 1987.
Greber et al., Stem Cells, 25:455-464, 2007.
Guide to Techniques in Mouse Development, 1993.
Harb et al., PLOS One, 20; 3(8):e3001, 2008.
In vitro Methods in Pharmaceutical Research, Academic Press, 1997.
Ishizaki, et al., Mol. Pharmacol., 57:976-983, 2000.
Jaeger et al., Development, 138(20):4363-74, October 2011
Jiang et al., 2012.
Jainchill et al., J. Virol., 4:549, 1969.
Keller et al., Curr. Opin. Cell Biol., 7:862-869, 1995.
Kim et al, Nature, 418:50-56, 2002.
Kirkeby et al., Generation of Regionally Specified Neural Progenitors and Functional Neurons from Human Embryonic Stem Cells under Defined Conditions, Cell Rep. 1(6):703-14, 2012.
Klimanskaya et al., Lancet., 365:P1636-1641, 2005.
Kodama et al., J. Cell. Physiol., 112:89, 1982.
Krencik and Zhang, Nature Protocols 6(11):1710-1717, 2011.
Krencik et al., Nature Biotechnology 29:528-534, 2011.
Kriks, et al., Dopamine Neurons derived from human ES cells efficiently engraft in animal models of Parkinson's disease, Nature. 480(7378):547-51, 2011.
Ludwig et al., Nat. Biotechnol., 24(2):185-187, 2006b.
Ludwig et al., Nat. Methods, 3(8):637-46, 2006a.
Manipulating the Mouse Embryo A Laboratory Manual, Second Edition, Cold Spring Harbor Laboratory Press, 1994.
Martin, Proc. Natl. Acad. Sci. USA, 78:7634, 1981.
Mouse Development, 1993.
Nakajima et al., Cancer Chemother. Pharmacol., 52:319-324, 2003.
Nagaoka et al., BMC Dev Biol., 10: 60, 2010.
Nakano et al., Science, 272, 722, 1996.
Ogawa et al., J. Cell Sci., 120:55-65, 2007.
Parkin controls dopamine utilization in human midbrain dopaminergic derived from induced pluripotent stem cells” Nat Commun., 3:668, 2012.
Perrier et al., Proc. Natl. Acad. Sci. USA, 101(34):12543-8, 2004.
Properties and uses of Embryonic Stem Cells: Prospects for Application to Human Biology and Gene Therapy, 1998.
Reubinoff et al., Nat. Biotechnol., 18:399-404, 2000.
Sasaki et al., Pharmacol. Ther., 93:225-232, 2002.
Schwartz et al., Methods 45(2): 142-158, 2008.
Smith, In: Origins and Properties of Mouse Embryonic Stem Cells, Annu. Rev. Cell. Dev. Biol., 2000.
Sterneckert et al., Stem Cells, 28:1772-1781, 2010.
Suzuki et al., Proc. Natl. Acad. Sci. USA, 103:10294-10299., 2006.
Tachibana et al. Cell. 2013 Jun. 6; 153(6):1228-38.
Takahashi and Yamanaka, Cell, 126:663-676, 2006.
Takahashi et al., Cell, 126(4):663-76, 2006.
Takahashi et al., Cell, 131:861-872, 2007.
Thomson and Marshall, Curr. Top. Dev. Biol., 38:133-165, 1998.
Thomson and Odorico, J. Trends. Biotechnol., 18:53B57, 2000.
Thomson et al. Proc. Natl. Acad. Scie. USA, 92:7844-7848, 1995.
Thomson et al., Science, 282:1145, 1998.
Watabe and Miyazono, Cell Res., 19:103-115, 2009.
Watanabe et al., Nature Neurosci., 8:288-296, 2005.
Xi et al., Stem Cells. 30(8):1655-63., 2012.
Xu et al., Cell Stem Cell, 3:196-206., 2008.
Xu et al., Nat. Biotechnol., 19:971-974, 2001.
Yakubov et al., Biochemical and Biophysical Research Communications 394: 189-193, 2010.
Ying et al., Cell, 115:281-292, 2003.
Young et al., Mol Ther. 22(8): 1530-43, 2014.
Yu and Thomson, Genes Dev. 22(15): 1987-97, 2008.
Yu et al., Science, 318:1917-1920, 2007.
Yu et al., Nature Medicine, 14: 1363, 2008.
Yu et al., Science, 324(5928): 797-801, 2009.
Blesa, J. and S. Przedborski, Parkinson's disease: animal models and dopaminergic cell vulnerability. Front Neuroanat, 2014. 8: p. 155.
Campos, F. L., et al., Rodent models of Parkinson's disease: beyond the motor symptomatology. Front Behav Neurosci, 2013. 7: p. 175.
Deumens, R., A. Blokland, and J. Prickaerts, Modeling Parkinson's disease in rats: an evaluation of 6-OHDA lesions of the nigrostriatal pathway. Exp Neurol, 2002. 175(2): p. 303-17.
Vermilyea, S. C. and M. E. Emborg, The role of nonhuman primate models in the development of cell-based therapies for Parkinson's disease. J Neural Transm (Vienna), 2018. 125(3): p. 365-384.
Wakeman et al., Stem Cell Reports. 2017 Jul. 11; 9(1):149-161.
Agarwala, S., Sanders, T. A., & Ragsdale, C. W. (2001). Sonic hedgehog control of size and shape in midbrain pattern formation. Science, 291(5511), 2147-2150. doi: 10.1126/science.1058624
Ahlskog, J. E., & Muenter, M. D. (2001). Frequency of levodopa-related dyskinesias and motor fluctuations as estimated from the cumulative literature. Mov Disord, 16(3), 448-458. doi:10.1002/mds.1090
Barbuti, P. A., Barker, R. A., Brundin, P., Przedborski, S., Papa, S. M., Kalia, L. V., . . . Committee, M. D.S.S.I. (2021). Recent Advances in the Development of Stem-Cell-Derived Dopaminergic Neuronal Transplant Therapies for Parkinson's Disease. Mov Disord. doi: 10.1002/mds.28628
Barker, R. A., & consortium, T. (2019). Designing stem-cell-based dopamine cell replacement trials for Parkinson's disease. Nat Med, 25(7), 1045-1053. doi: 10.1038/s41591-019-0507-2
Barker, R. A., Drouin-Ouellet, J., & Parmar, M. (2015). Cell-based therapies for Parkinson disease-past insights and future potential. Nat Rev Neurol, 11(9), 492-503. doi: 10.1038/nrneurol.2015.123
Brundin, P., Nilsson, O. G., Strecker, R. E., Lindvall, O., Astedt, B., & Bjorklund, A. (1986). Behavioural effects of human fetal dopamine neurons grafted in a rat model of Parkinson's disease. Exp Brain Res, 65(1), 235-240. doi: 10.1007/BF00243848
Bye, C. R., Thompson, L. H., & Parish, C. L. (2012). Birth dating of midbrain dopamine neurons identifies A9 enriched tissue for transplantation into parkinsonian mice. Exp Neurol, 236(1), 58-68. doi: 10.1016/j.expneurol.2012.04.002
Cardoso, T., Adler, A. F., Mattsson, B., Hoban, D. B., Nolbrant, S., Wahlestedt, J. N., . . . Parmar, M. (2018). Target-specific forebrain projections and appropriate synaptic inputs of hESC-derived dopamine neurons grafted to the midbrain of parkinsonian rats. J Comp Neurol, 526(13), 2133-2146. doi:10.1002/cne.24500
Carlsson, T., Carta, M., Munoz, A., Mattsson, B., Winkler, C., Kirik, D., & Bjorklund, A. (2009). Impact of grafted serotonin and dopamine neurons on development of L-DOPA-induced dyskinesias in parkinsonian rats is determined by the extent of dopamine neuron degeneration. Brain, 132(Pt 2), 319-335. doi: 10.1093/brain/awn305
Doi, D., Magotani, H., Kikuchi, T., Ikeda, M., Hiramatsu, S., Yoshida, K., . . . Takahashi, J. (2020). Preclinical study of induced pluripotent stem cell-derived dopaminergic progenitor cells for Parkinson's disease. Nat Commun, 11(1), 3369. doi: 10.1038/s41467-020-17165-w
Domanskyi, A., Alter, H., Vogt, M. A., Gass, P., & Vinnikov, I. A. (2014). Transcription factors Foxa1 and Foxa2 are required for adult dopamine neurons maintenance. Front Cell Neurosci, 8, 275. doi:10.3389/fncel.2014.00275
Dorsey, E. R., & Bloem, B. R. (2018). The Parkinson Pandemic-A Call to Action. JAMA Neurol, 75(1), 9-10. doi: 10.1001/jamaneurol.2017.3299
Doucet, G., Murata, Y., Brundin, P., Bosler, O., Mons, N., Geffard, M., . . . Bjorklund, A. (1989). Host afferents into intrastriatal transplants of fetal ventral mesencephalon. Exp Neurol, 106(1), 1-19. doi:10.1016/0014-4886(89)90139-8
Freed, C. R., Greene, P. E., Breeze, R. E., Tsai, W. Y., DuMouchel, W., Kao, R., . . . Fahn, S. (2001). Transplantation of embryonic dopamine neurons for severe Parkinson's disease. N Engl J Med, 344(10), 710-719. doi: 10.1056/NEJM200103083441002
Freeman, T. B., Olanow, C. W., Hauser, R. A., Nauert, G. M., Smith, D. A., Borlongan, C. V., . . . et al. (1995). Bilateral fetal nigral transplantation into the postcommissural putamen in Parkinson's disease. Ann Neurol, 38(3), 379-388. doi: 10.1002/ana.410380307
Freeman, T. B., Sanberg, P. R., Nauert, G. M., Boss, B. D., Spector, D., Olanow, C. W., & Kordower, J. H. (1995). The influence of donor age on the survival of solid and suspension intraparenchymal human embryonic nigral grafts. Cell Transplant, 4(1), 141-154. doi: 10.1016/0963-6897(94)00048-0
Ganat, Y. M., Calder, E. L., Kriks, S., Nelander, J., Tu, E. Y., Jia, F., . . . Studer, L. (2012). Identification of embryonic stem cell-derived midbrain dopaminergic neurons for engraftment. J Clin Invest, 122(8), 2928-2939. doi: 10.1172/JCI58767
Grealish, S., Diguet, E., Kirkeby, A., Mattsson, B., Heuer, A., Bramoulle, Y., . . . Parmar, M. (2014). Human ESC-derived dopamine neurons show similar preclinical efficacy and potency to fetal neurons when grafted in a rat model of Parkinson's disease. Cell Stem Cell, 15(5), 653-665. doi:10.1016/j.stem.2014.09.017
Grealish, S., Heuer, A., Cardoso, T., Kirkeby, A., Jonsson, M., Johansson, J., . . . Parmar, M. (2015). Monosynaptic Tracing using Modified Rabies Virus Reveals Early and Extensive Circuit Integration of Human Embryonic Stem Cell-Derived Neurons. Stem Cell Reports, 4(6), 975-983. doi:10.1016/j.stemcr.2015.04.011
Hagell, P., & Cenci, M. A. (2005). Dyskinesias and dopamine cell replacement in Parkinson's disease: a clinical perspective. Brain Res Bull, 68(1-2), 4-15. doi:10.1016/j.brainresbull.2004.10.013
Hagell, P., Piccini, P., Bjorklund, A., Brundin, P., Rehncrona, S., Widner, H., . . . Lindvall, O. (2002). Dyskinesias following neural transplantation in Parkinson's disease. Nat Neurosci, 5(7), 627-628. doi:10.1038/nn863
Hallett, P. J., Deleidi, M., Astradsson, A., Smith, G. A., Cooper, O., Osborn, T. M., . . . Isacson, O. (2015). Successful function of autologous iPSC-derived dopamine neurons following transplantation in a non-human primate model of Parkinson's disease. Cell Stem Cell, 16(3), 269-274. doi:10.1016/j.stem.2015.01.018
Hiller, B. M., Marmion, D. J., Gross, R. M., Thompson, C. A., Chavez, C. A., Brundin, P., . . . Kordower, J. H. (2020). Mitomycin-C treatment during differentiation of induced pluripotent stem cell-derived dopamine neurons reduces proliferation without compromising survival or function in vivo. Stem Cells Transl Med. doi: 10.1002/sctm.20-0014
Kee, N., Volakakis, N., Kirkeby, A., Dahl, L., Storvall, H., Nolbrant, S., . . . Perlmann, T. (2017). Single-Cell Analysis Reveals a Close Relationship between Differentiating Dopamine and Subthalamic Nucleus Neuronal Lineages. Cell Stem Cell, 20(1), 29-40. doi: 10.1016/j.stem.2016.10.003
Kim, T. W., Piao, J., Koo, S. Y., Kriks, S., Chung, S. Y., Betel, D., . . . Studer, L. (2021). Biphasic Activation of WNT Signaling Facilitates the Derivation of Midbrain Dopamine Neurons from hESCs for Translational Use. Cell Stem Cell, 28(2), 343-355 e345. doi: 10.1016/j.stem.2021.01.005
Kirkeby, A., Grealish, S., Wolf, D. A., Nelander, J., Wood, J., Lundblad, M., . . . Parmar, M. (2012). Generation of regionally specified neural progenitors and functional neurons from human embryonic stem cells under defined conditions. Cell Rep, 1(6), 703-714. doi:10.1016/j.celrep.2012.04.009
Kirkeby, A., Nolbrant, S., Tiklova, K., Heuer, A., Kee, N., Cardoso, T., . . . Parmar, M. (2017). Predictive Markers Guide Differentiation to Improve Graft Outcome in Clinical Translation of hESC-Based Therapy for Parkinson's Disease. Cell Stem Cell, 20(1), 135-148. doi: 10.1016/j.stem.2016.09.004
Kittappa, R., Chang, W. W., Awatramani, R. B., & Mckay, R. D. (2007). The foxa2 gene controls the birth and spontaneous degeneration of dopamine neurons in old age. PLOS Biol, 5(12), e325. doi:10.1371/journal.pbio.0050325
Kriks, S., Shim, J. W., Piao, J., Ganat, Y. M., Wakeman, D. R., Xie, Z., . . . Studer, L. (2011). Dopamine neurons derived from human ES cells efficiently engraft in animal models of Parkinson's disease. Nature, 480(7378), 547-551. doi: 10.1038/nature 10648
Lane, E. L., & Smith, G. A. (2010). Understanding graft-induced dyskinesia. Regen Med, 5(5), 787-797. doi: 10.2217/rme. 10.42
Li, J. Y., & Li, W. (2021). Postmortem Studies of Fetal Grafts in Parkinson's Disease: What Lessons Have We Learned? Front Cell Dev Biol, 9, 666675. doi: 10.3389/fcell.2021.666675
Lindvall, O., Brundin, P., Widner, H., Rehncrona, S., Gustavii, B., Frackowiak, R., . . . et al. (1990). Grafts of fetal dopamine neurons survive and improve motor function in Parkinson's disease. Science, 247(4942), 574-577. doi: 10.1126/science.2105529
Ma, Y., Feigin, A., Dhawan, V., Fukuda, M., Shi, Q., Greene, P., . . . Eidelberg, D. (2002). Dyskinesia after fetal cell transplantation for parkinsonism: a PET study. Ann Neurol, 52(5), 628-634. doi:10.1002/ana.10359
Morizane, A., Doi, D., & Takahashi, J. (2013). Neural induction with a dopaminergic phenotype from human pluripotent stem cells through a feeder-free floating aggregation culture. Methods Mol Biol, 1018, 11-19. doi: 10.1007/978-1-62703-444-9_2
Niclis, J. C., Gantner, C. W., Alsanie, W. F., McDougall, S. J., Bye, C. R., Elefanty, A. G., . . . Parish, C. L. (2017). Efficiently Specified Ventral Midbrain Dopamine Neurons from Human Pluripotent Stem Cells Under Xeno-Free Conditions Restore Motor Deficits in Parkinsonian Rodents. Stem Cells Transl Med, 6(3), 937-948. doi: 10.5966/sctm.2016-0073
Nouri, N., & Awatramani, R. (2017). A novel floor plate boundary defined by adjacent En1 and Dbx1 microdomains distinguishes midbrain dopamine and hypothalamic neurons. Development, 144(5), 916-927. doi: 10.1242/dev.144949
Olanow, C. W., Goetz, C. G., Kordower, J. H., Stoessl, A. J., Sossi, V., Brin, M. F., . . . Freeman, T. B. (2003). A double-blind controlled trial of bilateral fetal nigral transplantation in Parkinson's disease. Ann Neurol, 54(3), 403-414. doi: 10.1002/ana.10720
Olanow, C. W., Gracies, J. M., Goetz, C. G., Stoessl, A. J., Freeman, T., Kordower, J. H., . . . Obeso, J. A. (2009). Clinical pattern and risk factors for dyskinesias following fetal nigral transplantation in Parkinson's disease: a double blind video-based analysis. Mov Disord, 24(3), 336-343. doi:10.1002/mds.22208
Piao, J., Zabierowski, S., Dubose, B. N., Hill, E. J., Navare, M., Claros, N., . . . Tabar, V. (2021). Preclinical Efficacy and Safety of a Human Embryonic Stem Cell-Derived Midbrain Dopamine Progenitor Product, MSK-DA01. Cell Stem Cell, 28(2), 217-229 e217. doi:10.1016/j.stem.2021.01.004
Qiu, L., Liao, M. C., Chen, A. K., Wei, S., Xie, S., Reuveny, S., . . . Zeng, L. (2017). Immature Midbrain Dopaminergic Neurons Derived from Floor-Plate Method Improve Cell Transplantation Therapy Efficacy for Parkinson's Disease. Stem Cells Transl Med, 6(9), 1803-1814. doi: 10.1002/sctm.16-0470
Song, B., Cha, Y., Ko, S., Jeon, J., Lee, N., Seo, H., . . . Kim, K. S. (2020). Human autologous iPSC-derived dopaminergic progenitors restore motor function in Parkinson's disease models. J Clin Invest, 130(2), 904-920. doi: 10.1172/JCI130767
Steinbeck, J. A., & Studer, L. (2015). Moving stem cells to the clinic: potential and limitations for brain repair. Neuron, 86(1), 187-206. doi: 10.1016/j.neuron.2015.03.002
Takahashi, K., & Yamanaka, S. (2006). Induction of pluripotent stem cells from mouse embryonic and adult fibroblast cultures by defined factors. Cell, 126(4), 663-676. doi: 10.1016/j.cell.2006.07.024
Thompson, L., Barraud, P., Andersson, E., Kirik, D., & Bjorklund, A. (2005). Identification of dopaminergic neurons of nigral and ventral tegmental area subtypes in grafts of fetal ventral mesencephalon based on cell morphology, protein expression, and efferent projections. J Neurosci, 25(27), 6467-6477. doi: 10.1523/JNEUROSCI.1676-05.2005
Thompson, L. H., Kirik, D., & Bjorklund, A. (2008). Non-dopaminergic neurons in ventral mesencephalic transplants make widespread axonal connections in the host brain. Exp Neurol, 213(1), 220-228. doi: 10.1016/j.expneurol.2008.06.005
Thomson, J. A., Itskovitz-Eldor, J., Shapiro, S. S., Waknitz, M. A., Swiergiel, J. J., Marshall, V. S., & Jones, J. M. (1998). Embryonic stem cell lines derived from human blastocysts. Science, 282(5391), 1145-1147. doi:10.1126/science.282.5391.1145
Wakeman, D. R., Hiller, B. M., Marmion, D. J., McMahon, C. W., Corbett, G. T., Mangan, K. P., . . . Kordower, J. H. (2017). Cryopreservation Maintains Functionality of Human iPSC Dopamine Neurons and Rescues Parkinsonian Phenotypes In Vivo. Stem Cell Reports, 9(1), 149-161. doi:10.1016/j.stemcr.2017.04.033
Wallen, A., Zetterstrom, R. H., Solomin, L., Arvidsson, M., Olson, L., & Perlmann, T. (1999). Fate of mesencephalic AHD2-expressing dopamine progenitor cells in NURR1 mutant mice. Exp Cell Res, 253(2), 737-746. doi: 10.1006/excr. 1999.4691
Wianny, F., & Vezoli, J. (2017). Transplantation in the nonhuman primate MPTP model of Parkinson's disease: update and perspectives. Primate Biol, 4(2), 185-213. doi: 10.5194/pb-4-185-2017
Yin, D., Valles, F. E., Fiandaca, M. S., Forsayeth, J., Larson, P., Starr, P., & Bankiewicz, K. S. (2009). Striatal volume differences between non-human and human primates. J Neurosci Methods, 176(2), 200-205. doi: 10.1016/j.jneumeth.2008.08.027

What is claimed is:

1. A culture comprising midbrain dopaminergic (mDA) neuronal precursor cells generated by culturing human pluripotent cells in the presence of the following signaling modulators:
(a) a first inhibitor of Small Mothers Against Decapentaplegic (SMAD) signaling,
(b) at least one activator of Sonic hedgehog (SHH) signaling, and
(c) at least one activator of wingless (Wnt) signaling;
wherein the method does not comprise culturing the human pluripotent cells in the presence of a second inhibitor of Small Mothers Against Decapentaplegic (SMAD) signaling;
and wherein the human pluripotent cells are cultured under conditions to induce differentiation for from about 360 to about 456 hours and then refrigerating or cryopreserving the cells; and
wherein the midbrain dopaminergic precursor cells express both forkhead box protein A2 (FOXA2) and LIM homeobox transcription factor 1 (LMX1) (FOXA2⁺/LMX1⁺ cells).

2. The culture of claim 1, wherein the human pluripotent cells are cultured under conditions to induce differentiation for from about 384 to about 432 hours.

3. The culture of claim 1, wherein the mDA neuronal precursor cells do not express NURR1.

4. The culture of any one of claims 1-2, wherein the mDA neuronal precursor cells express forkhead box protein A2 (FOXA2), LIM homeobox transcription factor 1 (LMX1), and EN1.

5. The culture of claim 4, wherein the mDA neuronal precursor cells further express OTX2.

6. The culture of any one of claims 1-5, wherein forkhead box protein A2 (FOXA2) and LIM homeobox transcription factor 1 (LMX1) are co-expressed by from about 60% to about 100% or from about 85% to about 95% or more of the mDA neuronal precursor cells.

7. The culture of claim 6, wherein about 65-75% of the mDA neuronal precursor co-express both FOXA2 and LMX1.

8. The culture of any one of claims 1-7, wherein the midbrain dopaminergic precursor cells express FOXA2, LMX1A, ETV5, and EN1; and wherein the midbrain dopaminergic precursor cells do not express NURR1, TH, CALB1, BARHL1, or GRIK2.

9. The culture of any one of claims 1-8, wherein the mDA neuronal precursor cells comprise proliferating or dividing cells.

10. The culture of claim 9, wherein at least about 40% or more of the mDA neuronal precursor cells are proliferating or dividing.

11. The culture of claim 10, wherein about 50-75% of the mDA neuronal precursor cells are proliferating or dividing.

12. The culture of any one of claims 1-9, wherein the culture further comprises about 5% or less of serotonergic neuronal precursor cells.

13. The culture of claim 12, wherein the serotonergic neuronal precursor cells express BARLH1.

14. The culture of any one of claims 1-12, wherein the culture further comprises glial progenitor cells.

15. The culture of claim 14, wherein the glial progenitor cells express GLAST, SLC13A, CD44, and/or hGFAP.

16. The culture of any of claims 1-8, wherein the inhibitor of SMAD signaling is a BMP inhibitor.

17. The culture of claim 16, wherein the BMP inhibitor is LDN-193189, dorsomorphin, DMH-1, or noggin.

18. The culture of claim 17, wherein the BMP inhibitor is LDN-193189.

19. The culture of claim 18, wherein the LDN-193189 is present at a concentration of from about 0.2 μM to about 4 μM, more preferably from about 1 μM to about 4 μM.

20. The culture of claim 19, wherein the LDN-193189 is present at a concentration of from about 1 μM to about 3 μM.

21. The culture of claim 19, wherein the LDN-193189 is present at a concentration of from about 0.5 μM to about 4 μM.

22. The culture of claim 21, wherein the LDN-193189 is present at a concentration of from about 0.5 μM to about 2 μM. 23 The culture of claim 19, wherein the LDN-193189 is present at a concentration of from about 0.2 μM to about 4 μM.

24. The culture of claim 23, wherein the LDN-193189 is present at a concentration of from about 0.2 μM to about 2 μM.

25. The culture of any of claims 1-8, wherein the SMAD signaling inhibitor is a TGFβ inhibitor.

26. The culture of claim 25, wherein the TGFβ inhibitor is SB431542.

27. The culture of claim 26, wherein the SB431542 is present at a concentration of about 1-20 μM.

28. The culture of claim 26, wherein the SB431542 is present at a concentration of about 5-15 μM.

29. The culture of claim 26, wherein the SB431542 is present at a concentration of about 10 μM.

30. The culture of any one of claims 1-29, wherein the pluripotent cells are cultured with the inhibitor of SMAD on culture days 1-15, 1-16, or 1-17.

31. The culture of claim 30, wherein the pluripotent cells are cultured with the inhibitor of SMAD on culture days 1-17.

32. The culture of any one of claims 1-31, wherein the pluripotent cells are cultured with the inhibitor of SMAD substantially continuously or on a daily basis for 15, 16, or 17 days.

33. The culture of claim 32, wherein the pluripotent cells are cultured with the inhibitor of SMAD substantially continuously or on a daily basis for 17 days.

34. The culture of any one of claims 1-33, wherein the inhibitor of SMAD is present at a concentration of about 50-2000 or 50-500 nM.

35. The culture of claim 34, wherein the inhibitor of SMAD is present at a concentration of about 180-240 nM.

36. The culture of any one of claims 1-35, wherein the method further comprises contacting the pluripotent cells with a MEK inhibitor.

37. The culture of claim 36, wherein the MEK inhibitor is PD0325901.

38. The culture of claim 37, where the PD0325901 is present at a concentration of about 0.25-2.5 μM.

39. The culture of any one of claims 35-38, wherein the MEK inhibitor is contacted to the pluripotent cells for about 1-3 days, or on days 1-3, 2-4, 3-5, or on days 1, 2, 3, 4, or 5, after initiation of contact with the inhibitor of SMAD signaling.

40. The culture of claim 39, wherein the MEK inhibitor is contacted to the pluripotent cells from about 24 to about 48 hours after initiation of contact with the inhibitor of SMAD signaling.

41. The culture of any one of claims 36-40, wherein the MEK inhibitor is contacted to the pluripotent cells on a daily or substantially continual basis for about 3-4 days beginning about 1-2 days after initiation of contact with the inhibitor of SMAD signaling.

42. The culture of claim 41, wherein the MEK inhibitor is contacted to the pluripotent cells on days 2-5 or days 3-6 after initiation of contact with the inhibitor of SMAD signaling on day 1.

43. The culture of any one of claims 1-40, wherein the activator of Wnt signaling is a GSK3 inhibitor.

44. The culture of claim 43, wherein the GSK3 inhibitor is CHIR99021.

45. The culture of claim 44, wherein the CHIR99021 is present at a concentration of about 1.5-2 μM.

46. The culture of claim 44, wherein the CHIR99021 is present at a concentration of about 1.5-1.7 μM.

47. The culture of claim 45, wherein the CHIR99021 is present at a concentration of about 1.6-1.7 μM.

48. The culture of claim 45, wherein the CHIR99021 is present at a concentration of about 1.65 μM.

49. The culture of claim 44, wherein the CHIR99021 is present at a concentration of about 4-7 μM on days 9-17 after initiation of contact with the inhibitor of SMAD signaling.

50. The culture of any one of claims 1-49, wherein the activator of Wnt signaling is contacted to the pluripotent cells 1-3 days after initiation of contact with the inhibitor of SMAD signaling.

51. The culture of claim 50, wherein the activator of Wnt signaling is contacted to the pluripotent cells within 24-48 hours after initiation of contact with the inhibitor of SMAD signaling.

52. The culture of any one of claims 1-51, wherein the pluripotent cells are cultured with the activator of Wnt signaling substantially continuously or on a daily basis for 14, 15, or about 16 days.

53. The culture of any one of claims 1-52, wherein the activator of Wnt signaling is contacted to the pluripotent cells on days 2-17 after initiation of contact with the inhibitor of SMAD signaling.

54. The culture of any one of claims 1-52, wherein the activator of SHH signaling is purmorphamine or C25II Shh.

55. The culture of claim 54, wherein the method further comprises contacting the pluripotent cells with two activators of SHH signaling.

56. The culture of claim 55, wherein the two activators of SHH signaling are purmorphamine and C25II Shh.

57. The culture of any one of claims 1-56, wherein the at least one activator of SHH signaling is contacted to the pluripotent cells on the same day as initiation of contact with the inhibitor of SMAD signaling or within 24-48 hours after initiation of contact with the inhibitor of SMAD signaling.

58. The culture of claim 57, wherein the at least one activator of SHH signaling is contacted to the pluripotent cells on days 1-7 with or after initiation of contact with the inhibitor of SMAD signaling.

59. The culture of any one of claims 1-58, wherein the method further comprises contacting the pluripotent cells with FGF-8.

60. The culture of claim 59, wherein the FGF-8 is not contacted to the pluripotent cells on the same day as the initiation of contact with the inhibitor of SMAD signaling.

61. The culture of any one of claims 59-60, wherein the FGF-8 is contacted with the pluripotent cells on days 9-17 or 11-17 after initiation of contact with the inhibitor of SMAD signaling.

62. The culture of any one of claims 59-61, wherein the FGF-8 is present at a concentration of about 50-200 ng/mL.

63. The culture of any one of claims 1-62, wherein the pluripotent cells comprise an antibiotic resistance transgene under the control of a neuronal promoter.

64. The culture of any one of claims 1-63, wherein the method further comprises selecting for neural cells or midbrain DA neurons derived from the pluripotent cells by contacting cells with an antibiotic, a chemotherapeutic, a DNA crosslinker, a DNA synthesis inhibitors, or a mitotic inhibitor.

65. The culture of any one of claims 1-63, wherein the method further comprises contacting the pluripotent cells with an antibiotic or a chemotherapeutic.

66. The culture of any one of claims 64-65, wherein the chemotherapeutic is mitomycin C.

67. The culture of claim 66, wherein the mitomycin C is contacted with the pluripotent cells on days 27, 28, 29, and/or 30 after initiation of contact with the inhibitor of SMAD signaling.

68. The culture of any one of claims 64-65, wherein the antibiotic is G418 (geneticin).

69. The culture of any one of claims 1-68, wherein the method further comprises culturing or incubating the pluripotent cells in a media comprising a ROCK inhibitor prior to initiation of contact with the inhibitor of SMAD signaling.

70. The culture of any one of claims 1-69, wherein the method further comprises contacting the pluripotent cells with blebbistatin.

71. The culture of any one of claims 1-70, wherein the blebbistatin is contacted with the cells on day 5 and day 17 of differentiation.

72. The culture of any one of claims 1-71, wherein the mDA dopaminergic precursor cells do not express NURR1, MAP2, or TH.

73. The culture of any one of claims 1-72, wherein the mDA dopaminergic precursor cells express EN1.

74. The culture of any one of claims 1-72, wherein the mDA dopaminergic precursor cells express GBX2, OTX1, OTX2, ETV5, CORIN, and/or DCX.

75. The culture of any one of claims 1-73, wherein the pluripotent cells are human induced pluripotent stem (iPS) cells.

76. The culture of any one of claims 1-75, wherein the LMX1 is LIM homeobox transcription factor 1 alpha (LMX1A).

77. The culture of any one of claims 1-76, wherein the method further comprises incubating the human pluripotent cells in the presence of a DNase or an endonuclease.

78. The culture of claim 77, wherein the endonuclease is DNase I or Benzonase®.

79. The culture of claim 78, wherein the DNase I or Benzonase® is present at a concentration of about 10-20 U/mL.

80. The culture of claim 79, wherein the DNase I or Benzonase® is present at a concentration of about 10-15 U/mL.

81. The culture of any one of claims 77-79, wherein the human pluripotent cells are cultured in the presence of an endonuclease on at least one of days 4-6 after initiation of contact with the inhibitor of SMAD signaling.

82. The culture of any one of claims 77-79, wherein the human pluripotent cells are cultured in the presence of an endonuclease on day 5 after initiation of contact with the inhibitor of SMAD signaling.

83. The culture of any one of claims 1-82, where the culture is comprised in a container means.

84. The culture of any one of claims 1-83, wherein the midbrain dopaminergic neuronal precursor cells are comprised in a pharmaceutical preparation.

85. The culture of claim 5, wherein the pharmaceutical preparation is formulated for injection.

86. The culture of any one of claims 1-85, wherein the culture comprises from about 2,500 cells/μL to about 150,000 cells/μL, from about 2,500 cells/μL to about 100,000 cells/μL, from about 10,000 cells/μL to about 150,000 cells/μL, from about 40,000 cells/μL to about 100,000 cells/μL, or about 15,000-45,000 cells/μL midbrain dopaminergic neuronal precursor cells.

87. The culture of any one of claims 1-86, wherein about 10% or less, more preferably about 7% or less of the cells in the culture are serotonergic precursor cells.

88. The culture of claim 87, wherein about 5% or less of the cells in the culture are serotonergic precursor cells.

89. The culture of claim 87, wherein about 5% or less of the cells in the culture express SERT and TPH2.

90. The culture of any one of claims 1-89, wherein about 0.1-5% or less of the cells in the culture express FOXG1, and/or wherein about 0.1-5% or less of the cells in the culture express PAX6.

91. The culture of claim 90, wherein less than about 1% of the cells in the culture express FOXG1, and/or wherein less than about 1% of the cells in the culture express PAX6.

92. A method of treating a disease in a mammalian subject comprising administering to the subject a therapeutically effective amount of the culture of any one of claims 1-91, preferably wherein the culture is administered to the brain of the subject.

93. The method of claim 92, wherein the mammalian subject is a human.

94. The method of claim 93, wherein the disease is a disease of the central nervous system (CNS).

95. The method of claim 94, wherein the disease is Parkinson's disease (PD) or a Parkinson-plus syndrome (PPS).

96. The method of any one of claims 92-95, wherein the culture comprises mDA precursor cells that express engrailed, but do not express NURR1.

97. The method of any one of claim 96, wherein the culture is administered to the striatum, such as the putamen or substantia nigra, of the subject.

98. The method of claim 97, wherein the culture is administered to more than one location into the striatum or putamen of the subject.

99. The method of claim 97, wherein the culture is administered at multiple sites and/or at multiple needle tracts into the striatum or putamen of the subject.

100. The method of claim 96, wherein the culture is comprised in a pharmaceutical composition.

101. The method of claim 100, comprises a hyaluronic acid matrix.

102. The method of any one of claims 92-101, wherein the culture comprises from about 1e6 to about 25e6, more preferably from about 3e6 to about 9e6 cells.

103. The method of any one of claims 92-102, wherein the culture comprises from about 2,500 cells/μL to about 150,000 cells/μL.

104. The method of claim 103, wherein the culture comprises from about 10,000 cells/μL to about 150,000 cells/μL.

105. The method of claim 103, wherein the culture comprises from about 40,000 cells/μL to about 100,000 cells/μL.

106. The method of any one of claims 92-105, wherein the subject has Parkinson's disease and wherein the subject exhibits improvement in at least one motor symptom after the administration of the culture.

107. The method of claim 106, wherein the subject exhibits a reduction in one or more of tremor, muscle rigidity, slowness of movement, falls, dizziness, movement freezing, muscle cramps, or dystonia.

108. The method of any one of claims 92-107, wherein the midbrain dopaminergic precursor cells at least partially reinnervate the striatum or putamen of the subject.

109. The method of any one of claims 92-108, wherein the midbrain dopaminergic precursor cells exhibit limited proliferation after the administration.

110. The method of any one of claims 92-109, wherein about 5% or less the cells in the cell culture are serotonergic cells or serotonergic precursor cells.

111. The method of any one of claims 92-110, wherein at least 80% of administered cells differentiate into differentiated cells that express both FOXA2 and LMX1.

112. The method of claim 111, wherein at least 85% of the differentiated cells express both FOXA2 and LMX1.

113. The method of any one of claims 92-112, wherein at least about 60% of the administered cells express both FOXA2 and LMX1.

114. The method of any one of claims 92-113, wherein the culture is cryogenically frozen prior to the administering.

115. The method of claim 114, wherein the culture is cryogenically frozen in liquid nitrogen prior to the administering.

116. The method of any one of claim 111, wherein the differentiated cells expressing FOXA2 and LMX1 further express at least one marker selected from the group consisting of engrailed (EN1), tyrosine kinase (TH), orthodenticle homeobox 2 (OTX2), nuclear receptor related 1 protein (NURR1), Neuron-specific class III beta-tubulin (Tuj1), TTF3, paired-like homeodomain 3 (PITX3), achaete-scute complex (ASCL), early B-cell factor 1 (EBF-1), early B-cell factor 3 (EBF-3), transthyretin (TTR), synapsin, dopamine transporter (DAT), and G-protein coupled, inwardly rectifying potassium channel (Kir3.2/GIRK2), CD142, DCSM1, CD63 and CD99.

117. The method of claim 116, wherein the differentiated cells expressing FOXA2 and LMX1, or FOXA2 and TH, further express engrailed, PITX3, and NURR1.

118. The method of any one of claims 111-116, wherein about 10-25% of the cells in the cell culture co-express FOXA2 and tyrosine hydroxylase (TH).

119. The method of any one of claims 92-118, wherein the pluripotent cells are human induced pluripotent stem (iPS) cells.

120. The method of any one of claims 92-119, wherein the LMX1 is LIM homeobox transcription factor 1 alpha (LMX1A).

121. The method of any one of claims 92-120, wherein less than about 1%, preferably less than 0.5%, of the cells in the cell composition are serotonergic cells.

122. The method of any one of claims 92-121, wherein the administration does not result in host gliosis.

123. The method of any one of claims 92-122, wherein the administration results in no or essentially no growth or proliferation of non-neuronal cells in the brain of the subject.

124. The method of any one of claims 92-123, wherein the administration results in the engraftment of the mDA precursor cells in the brain of the subject and/or innervation of at least part of the brain of the subject by the mDA precursor cells.

125. The method of any one of claims 92-124, wherein the administration is via injection.

126. The method of claim 125, wherein the injection is stereotaxic injection.

127. An in vitro method for preparing a cell composition comprising human cells that express both forkhead box protein A2 (FOXA2) and LIM homeobox transcription factor 1 (LMX1) (FOXA2⁺/LMX1⁺ cells) comprising culturing human pluripotent cells in the presence of the following signaling modulators:
(a) a first inhibitor of Small Mothers Against Decapentaplegic (SMAD) signaling,
(b) at least one activator of Sonic hedgehog (SHH) signaling, and
(c) at least one activator of wingless (Wnt) signaling;
wherein the method does not comprise culturing the human pluripotent cells in the presence of a second inhibitor of Small Mothers Against Decapentaplegic (SMAD) signaling;
and wherein the human pluripotent cells are cultured under conditions to induce differentiation for from about 360 to about 456 hours and then refrigerating or cryopreserving the cells.

128. The method of claim 127, wherein the human pluripotent cells are cultured under conditions to induce differentiation for from about 384 to about 432 hours.

129. The method of claim 127, wherein the human cells do not express NURR1.

130. The method of any one of claims 127-128, wherein the human cells express forkhead box protein A2 (FOXA2), LIM homeobox transcription factor 1 (LMX1), and Engrailed Homeobox 1 (EN1).

131. The method of claim 130, wherein the human cells further express OTX2.

132. The method of any one of claims 127-130, wherein forkhead box protein A2 (FOXA2) and LIM homeobox transcription factor 1 (LMX1) are co-expressed by from about 65% to about 85% or more of the human cells.

133. The method of any of claims 127-132, wherein the inhibitor of SMAD signaling is a BMP inhibitor.

134. The method of claim 133, wherein the BMP inhibitor is LDN-193189, dorsomorphin, DMH-1, or noggin.

135. The method of claim 134, wherein the BMP inhibitor is LDN-193189.

136. The method of claim 135, wherein the LDN-193189 is present at a concentration of from about 0.2 μM to about 4 μM.

137. The method of claim 136, wherein the LDN-193189 is present at a concentration of from about 1 μM to about 3 μM.

138. The method of claim 136, wherein the LDN-193189 is present at a concentration of from about 0.5 μM to about 4 μM.

139. The method of claim 138, wherein the LDN-193189 is present at a concentration of from about 0.5 μM to about 2 μM.

140. The method of claim 136, wherein the LDN-193189 is present at a concentration of from about 0.2 μM to about 4 μM.

141. The method of claim 140, wherein the LDN-193189 is present at a concentration of from about 0.2 μM to about 2 μM.

142. The method of any of claims 127-132, wherein the SMAD signaling inhibitor is a TGFβ inhibitor.

143. The method of claim 142, wherein the TGFβ inhibitor is SB431542.

144. The method of claim 143, wherein the SB431542 is present at a concentration of about 1-20 μM.

145. The method of claim 143, wherein the SB431542 is present at a concentration of about 5-15 μM.

146. The method of claim 143, wherein the SB431542 is present at a concentration of about 10 μM.

147. The method of any one of claims 127-146, wherein the pluripotent cells are cultured with the inhibitor of SMAD on culture days 1-15, 1-16, or 1-17.

148. The method of claim 147, wherein the pluripotent cells are cultured with the inhibitor of SMAD on culture days 1-17.

149. The method of any one of claims 127-148, wherein the pluripotent cells are cultured with the inhibitor of SMAD substantially continuously or on a daily basis for 15, 16, or 17 days.

150. The method of claim 149, wherein the pluripotent cells are cultured with the inhibitor of SMAD substantially continuously or on a daily basis for 17 days.

151. The method of any one of claims 127-150, wherein the inhibitor of SMAD is present at a concentration of about 50-2000 or 50-500 nM.

152. The method of claim 151, wherein the inhibitor of SMAD is present at a concentration of about 180-240 nM.

153. The method of any one of claims 127-152, wherein the method further comprises contacting the pluripotent cells with a MEK inhibitor.

154. The method of claim 153, wherein the MEK inhibitor is PD0325901.

155. The method of claim 154, where the PD0325901 is present at a concentration of about 0.25-2.5 μM.

156. The method of any one of claims 152-155, wherein the MEK inhibitor is contacted to the pluripotent cells for about 1-3 days, or on days 1-3, 2-4, 3-5, or on days 1, 2, 3, 4, or 5, after initiation of contact with the inhibitor of SMAD signaling.

157. The method of claim 156, wherein the MEK inhibitor is contacted to the pluripotent cells from about 24 to about 48 hours after initiation of contact with the inhibitor of SMAD signaling.

158. The method of any one of claims 153-157, wherein the MEK inhibitor is contacted to the pluripotent cells on a daily or substantially continual basis for about 3-4 days beginning about 1-2 days after initiation of contact with the inhibitor of SMAD signaling.

159. The method of claim 158, wherein the MEK inhibitor is contacted to the pluripotent cells on days 2-5 or days 3-6 after initiation of contact with the inhibitor of SMAD signaling on day 1.

160. The method of any one of claims 127-157, wherein the activator of Wnt signaling is a GSK3 inhibitor.

161. The method of claim 160, wherein the GSK3 inhibitor is CHIR99021.

162. The method of claim 161, wherein the CHIR99021 is present at a concentration of about 1.5-1.7 μM.

163. The method of claim 162, wherein the CHIR99021 is present at a concentration of about 1.6-1.7 μM.

164. The method of claim 162, wherein the CHIR99021 is present at a concentration of 1.65 μM.

165. The method of claim 161, wherein the CHIR99021 is present at a concentration of about 4-7 μM on days 9-17 after initiation of contact with the inhibitor of SMAD signaling.

166. The method of any one of claims 127-165, wherein the activator of Wnt signaling is contacted to the pluripotent cells 1-3 days after initiation of contact with the inhibitor of SMAD signaling.

167. The method of claim 166, wherein the activator of Wnt signaling is contacted to the pluripotent cells within 24-48 hours after initiation of contact with the inhibitor of SMAD signaling.

168. The method of any one of claims 127-167, wherein the pluripotent cells are cultured with the activator of Wnt signaling substantially continuously or on a daily basis for 14, 15, or about 16 days.

169. The method of any one of claims 127-168, wherein the activator of Wnt signaling is contacted to the pluripotent cells on days 2-17 after initiation of contact with the inhibitor of SMAD signaling.

170. The method of any one of claims 127-168, wherein the activator of SHH signaling is purmorphamine or C25II Shh.

171. The method of claim 170, wherein the method further comprises contacting the pluripotent cells with two activators of SHH signaling.

172. The method of claim 171, wherein the two activators of SHH signaling are purmorphamine and C25II Shh.

173. The method of any one of claims 127-172, wherein the at least one activator of SHH signaling is contacted to the pluripotent cells on the same day as initiation of contact with the inhibitor of SMAD signaling or within 24-48 hours after initiation of contact with the inhibitor of SMAD signaling.

174. The method of claim 173, wherein the at least one activator of SHH signaling is contacted to the pluripotent cells on days 1-7 with or after initiation of contact with the inhibitor of SMAD signaling.

175. The method of any one of claims 127-174, wherein the method further comprises contacting the pluripotent cells with FGF-8.

176. The method of claim 175, wherein the FGF-8 is not contacted to the pluripotent cells on the same day as the initiation of contact with the inhibitor of SMAD signaling.

177. The method of any one of claims 175-176, wherein the FGF-8 is contacted with the pluripotent cells on days 9-17 or 11-17 after initiation of contact with the inhibitor of SMAD signaling.

178. The method of any one of claims 175-177, wherein the FGF-8 is present at a concentration of about 50-200 ng/mL.

179. The method of any one of claims 127-178, wherein the pluripotent cells comprise an antibiotic resistance transgene under the control of a neuronal promoter.

180. The method of any one of claims 127-179, wherein the method further comprises selecting for neural cells or midbrain DA neurons derived from the pluripotent cells by contacting cells with an antibiotic, a chemotherapeutic, a DNA crosslinker, a DNA synthesis inhibitors, or a mitotic inhibitor.

181. The method of any one of claims 127-179, wherein the method further comprises contacting the pluripotent cells with an antibiotic or a chemotherapeutic.

182. The method of any one of claims 180-181, wherein the chemotherapeutic is mitomycin C.

183. The method of claim 182, wherein the mitomycin C is contacted with the pluripotent cells on days 27, 28, 28, and/or 29 after initiation of contact with the inhibitor of SMAD signaling.

184. The method of any one of claims 180-181, wherein the antibiotic is G418 (geneticin).

185. The method of any one of claims 127-184, wherein the method further comprises culturing or incubating the pluripotent cells in a media comprising a ROCK inhibitor prior to initiation of contact with the inhibitor of SMAD signaling.

186. The method of any one of claims 127-185, wherein the method further comprises contacting the pluripotent cells with blebbistatin.

187. The method of any one of claims 127-186, wherein the blebbistatin is contacted with the cells on day 5 and day 17 of differentiation.

188. The method of any one of claim 127-141 or 147-187, wherein at least 40% of the human pluripotent cells differentiate and express both FOXA2 and LMX1.

189. The method of claim 188, wherein at least 60% of the human pluripotent cells differentiate and express both FOXA2 and LMX1.

190. The method of claim 189, wherein at least 80% of the human pluripotent cells differentiate and express both FOXA2 and LMX1.

191. The method of claim 189, wherein at least 85% of the human pluripotent cells differentiate and express both FOXA2 and LMX1.

192. The method of any one of claim 127-141 or 147-187, wherein about 10-25% of the human pluripotent cells differentiate and express both FOXA2 and tyrosine hydroxylase (TH).

193. The method of any one of claims 127-192, wherein the pluripotent cells are human induced pluripotent stem (iPS) cells.

194. The method of any one of claims 127-193, wherein the LMX1 is LIM homeobox transcription factor 1 alpha (LMX1A).

195. The method of any one of claims 188-194, wherein the differentiated cells expressing FOXA2 and LMX1, or FOXA2 and TH, further express at least one marker selected from the group consisting of EN1, orthodenticle homeobox 2 (OTX2), Neuron-specific class III beta-tubulin (Tuj1), TTF3, paired-like homeodomain 3 (PITX3), achaete-scute complex (ASCL), early B-cell factor 1 (EBF-1), early B-cell factor 3 (EBF-3), transthyretin (TTR), synapsin, dopamine transporter (DAT), and G-protein coupled, inwardly rectifying potassium channel (Kir3.2/GIRK2), CD142, DCSM1, CD63 and CD99.

196. The method of any one of claims 127-194, wherein the FOXA2⁺/LMX1⁺ cells further express engrailed (EN1).

197. The method of any one of claims 127-194, wherein the FOXA2⁺/LMX1⁺ cells further express EN1, Pax8, and ETV5.

198. The method of any one of claims 127-197, wherein the FOXA2⁺/LMX1⁺ cells do not express NURR1.

199. The method of any one of claim 197, wherein the FOXA2⁺/LMX1⁺ cells express GBX2, OTX1, OTX2, ETV5, CORIN, and DCX.

200. The method of any one of claims 127-196, wherein 5% or less of the cells in the cell composition are serotonergic cells.

201. The method of any one of claims 127-200, wherein the method further comprises incubating human pluripotent cells in the presence of a DNase or an endonuclease.

202. The method of claim 201, wherein the endonuclease is DNase I or Benzonase®.

203. The method of claim 202, wherein the DNase I or Benzonase® is present at a concentration of about 10-20 U/mL.

204. The method of claim 203, wherein the DNase I or Benzonase® is present at a concentration of about 10-15 U/mL.

205. The method of any one of claims 201-203, wherein the human pluripotent cells are cultured in the presence of an endonuclease on at least one of days 4-6 after initiation of contact with the inhibitor of SMAD signaling.

206. The method of any one of claims 201-203, wherein the human pluripotent cells are cultured in the presence of an endonuclease on day 5 after initiation of contact with the inhibitor of SMAD signaling.

207. A method of screening a test compound comprising:
(a) contacting FOXA2⁺/LMX1A+ cells differentiated by the method of any one of claims 127-206 or the mDA precursor cells of any one of claims 1-86 with the test compound, and
(b) measuring the function, physiology, or viability of the cells.

208. The method of claim 207, wherein said measuring comprises testing for a toxicological response or an altered electrophysiological responses of the cells.

209. The method of any one of claims 207-208, wherein the cells are midbrain dopaminergic neurons or midbrain dopaminergic neuronal precursor cells.
